(19) United States
(12) Patent Application Publication
(10) Pub. No.: US 2022/0112209 A1
(43) Pub. Date: Apr. 14, 2022

(54) TETRACARBOXYLIC DIANHYDRIDE, POLYIMIDE PRECURSOR RESIN, POLYIMIDE, POLYIMIDE PRECURSOR RESIN SOLUTION, POLYIMIDE SOLUTION, AND POLYIMIDE FILM

(71) Applicant: ENEOS CORPORATION, Tokyo (JP)

(72) Inventors: Shinichi KOMATSU, Tokyo (JP); Daisuke WATANABE, Tokyo (JP)

(73) Assignee: ENEOS CORPORATION, Tokyo (JP)

(21) Appl. No.: 17/264,447

(22) PCT Filed: Aug. 1, 2019
(86) PCT No.: PCT/JP2019/030148

§ 371 Date: Jan. 29, 2021

(30) Foreign Application Priority Data
Aug. 3, 2018 (JP) 2018-147029
Dec. 26, 2018 (JP) 2018-242513

(51) Int. Cl. C07D 493/10; C08G 73/10; C08J 5/18

(52) U.S. Cl. CPC C07D 493/10; C08J 2379/08; C08J 5/18; C08G 73/1007

(57) ABSTRACT

A tetracarboxylic dianhydride that is a mixture of stereoisomers of a compound represented by the following general formula (1):

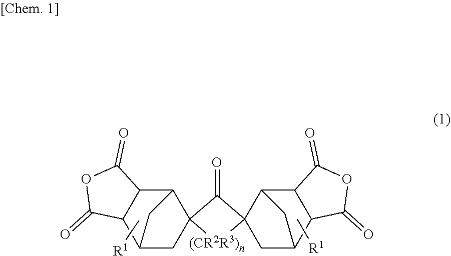

[in the formula (1), R¹, R², and R³ each independently represent a hydrogen atom or the like and n is an integer of 0 to 12], wherein a content of an isomer (A) represented by a specific general formula is 40% by mol to 98% by mol relative to a total amount of the stereoisomers, a content of an isomer (B) represented by a specific general formula is 2% by mol to 60% by mol relative to the total amount of the stereoisomers, and a summed amount of the isomers (A) and (B) is 42% by mol or more relative to the total amount of the stereoisomers.

### TECHNICAL FIELD

The present invention relates to a tetracarboxylic dianhydride, a polyimide precursor resin, a polyimide, a polyimide precursor resin solution, a polyimide solution, and a polyimide film.

### BACKGROUND ART

Conventionally, polyimides have attracted attention as a material which has a high heat resistance and which is lightweight and flexible. In the field of such polyimides, polyimides have been required in recent years having heat resistance and sufficient light transmittance usable in glass alternative application and the like, and various polyimides have been developed. For example, International Publication No. WO2011/099518 (PTL 1) discloses a polyimide which has a repeating unit represented by a specific general formula. Such a polyimide has sufficient heat resistance and light transmittance.

### CITATION LIST

### Patent Literature

[PTL 1] International Publication No. WO2011/099518

### SUMMARY OF INVENTION

### Technical Problem

The polyimide described in PTL 1 has sufficient heat resistance and light transmittance as mentioned above. However, in the field of polyimides, the advent of polyimides that can have lower linear expansion coefficient and dielectric dissipation factor while maintaining heat resistance and transparency to the same extent as those of the polyimide described in PTL 1 so as to be provided with properties depending on their applications has been desired.

The present invention has been made in view of the above-described problems of the conventional technique, and an object of the present invention is to provide a tetracarboxylic dianhydride that can be used as a raw material monomer for producing a polyimide that can have lower linear expansion coefficient and dielectric dissipation factor while having sufficiently high levels of heat resistance and transparency; a polyimide precursor resin that can be efficiently produced by using the tetracarboxylic dianhydride and can be used for producing a polyimide that can have lower linear expansion coefficient and dielectric dissipation factor while having sufficiently high levels of heat resistance and transparency; and a polyimide that can have lower linear expansion coefficient and dielectric dissipation factor while having sufficiently high levels of heat resistance and transparency. In addition, the present invention aims to provide a polyimide precursor resin solution comprising the polyimide precursor resin; a polyimide solution comprising the polyimide; and a polyimide film that is obtained by using these solutions.

### Solution to Problem

A compound produced in Example 2 of PTL 1 and represented by the following formula (A):

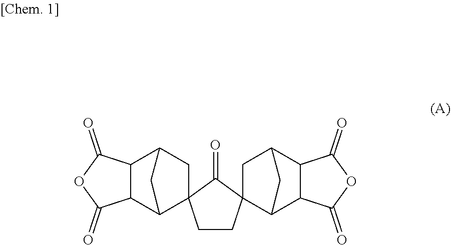 [SMILES: O=C1OC(=O)C2C1C1CC2C2(CCC3(CC4CC3C3C(=O)OC(=O)C43)C2=O)C1]

(norbornane-2-spiro-2′-cyclopentanone-5′-spiro-2″-nor bornane-5,5″,6,6″-tetracarboxylic dianhydride: CpODA) has a structure in which two norbornane rings are spiro-bonded to a cycloalkanone ring (cyclopentanone). In the case of the compound represented by the formula (A), the two norbornane rings in the compound can be arranged at cis or trans positions relative to the cycloalkanone ring and a carbonyl group (C═O) in the cycloalkanone ring can have an exo or endo three-dimensional configuration relative to each of the two norbornane rings in the compound. Moreover, it is known that the acid dianhydride ring bonded to the norbornane rings has an exo three-dimensional configuration (exo configuration) relative to the methylene head of norbornane (the bridgehead position of norbornane). Hence, it is known that the compound represented by the formula (A) includes six isomers represented by the following formula:

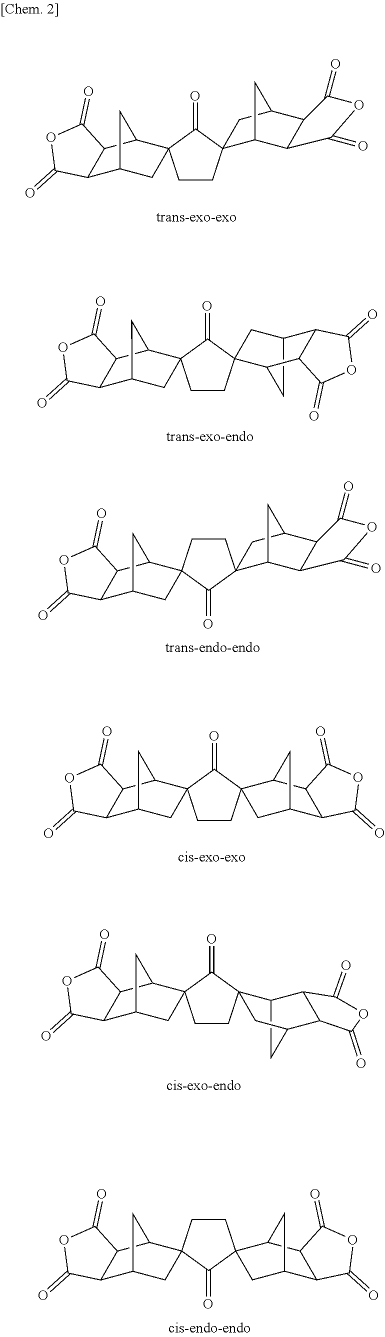 [SMILES: O=C1OC(=O)C2C1C1CC2C2(CCC3(CC4CC3C3C(=O)OC(=O)C43)C2=O)C1 ; O=C1OC(=O)C2C1C1CC2C2(CCC3(CC4CC3C3C(=O)OC(=O)C43)C2=O)C1 ; O=C1OC(=O)C2C1C1CC2C2(CCC3(CC4CC3C3C(=O)OC(=O)C43)C2=O)C1 ; O=C1OC(=O)C2C1C1CC2C2(CCC3(CC4CC3C3C(=O)OC(=O)C43)C2=O)C1 ; O=C1OC(=O)C2C1C1CC2C2(CCC3(CC4CC3C3C(=O)OC(=O)C43)C2=O)C1 ; O=C1OC(=O)C2C1C1CC2C2(CCC3(CC4CC3C3C(=O)OC(=O)C43)C2=O)C1]

(the notations of exo and endo in the above formula indicate the three-dimensional configurations of the carbonyl group (C═O) in the cycloalkanone ring relative to each of the left and right norbornane rings) (see Synthesis Example 2 of PTL 1). Regarding these six isomers, in the case where a method as actually employed in Synthesis Example 1, Example 1, and Example 2 of PTL 1 is used, the content ratio of each of the trans-exo-exo isomer and the cis-exo-exo isomer in a mixture of stereoisomers of the compound represented by the formula (A) is approximately 0.4% by mol, and the summed amount of the trans-exo-exo isomer and the cis-exo-exo isomer is less than 1% by mol. For this reason, in the case where the producing method as verified in PTL 1 is employed, the summed amount of the trans-exo-exo isomer and the cis-exo-exo isomer in the mixture of stereoisomers of the compound represented by the formula (A) becomes a trace amount (less than 1% by mol), so that only these two isomers cannot be separated from and taken out of the six isomers through crystallization or the like, and it has been impossible to produce such an mixture of isomers that the summed amount of the trans-exo-exo isomer and the cis-exo-exo isomer exceeds 40% by mol. Hence, regarding the compound represented by the formula (A), it has conventionally been impossible to obtain such an isomeric mixture that contains the trans-exo-exo isomer and the cis-exo-exo isomer at sufficiently high concentrations.

In view of this, the present inventors conducted earnest studies to make it possible to produce the trans-exo-exo isomer and the cis-exo-exo isomer at high concentration, and consequently found that preparation conditions of tetracarboxylic dianhydride that allow the content ratios of the trans-exo-exo isomer and the cis-exo-exo isomer to be at sufficiently high concentration in a mixture of stereoisomers of the compound represented by the above-described formula (A). Then, the present inventors applied such preparation conditions and found that, surprisingly, a tetracarboxylic dianhydride obtained in a mixture of stereoisomers of a compound represented by the following general formula (1) by setting a content of an isomer (A) represented by the following general formula (2) to 40% by mol to 98% by mol relative to a total amount of the stereoisomers in the mixture, setting a content of an isomer (B) represented by the following general formula (3) to 2% by mol to 60% by mol relative to the total amount of the stereoisomers in the mixture, and setting a summed amount of the isomers (A) and (B) to 42% by mol or more relative to the total amount of the stereoisomers in the mixture can be used as a raw material monomer for producing a polyimide that can have lower linear expansion coefficient and dielectric dissipation factor while having sufficiently high levels of heat resistance and transparency. This finding has led to the completion of the present invention.

Specifically, a tetracarboxylic dianhydride of the present invention is a mixture of stereoisomers of a compound represented by the following general formula (1):

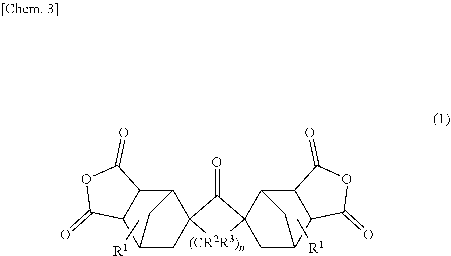

[in the formula (1), R¹, R², and R³ each independently represent one selected from the group consisting of a hydrogen atom, alkyl groups having 1 to 10 carbon atoms, and a fluorine atom, and n is an integer of 0 to 12], wherein

a content of an isomer (A) represented by the following general formula (2):

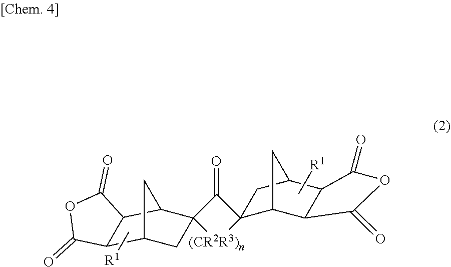

[R¹, R², R³, and n in the formula (2) have the same definitions as those of R¹, R², R³, and n in the general formula (1), respectively] is 40% by mol to 98% by mol relative to a total amount of the stereoisomers,

a content of an isomer (B) represented by the following general formula (3):

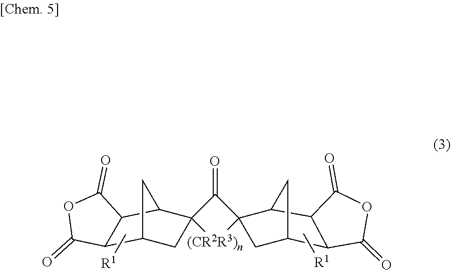

[R¹, R², R³, and n in the formula (3) have the same definitions as those of R¹, R², R³, and n in the general formula (1), respectively] is 2% by mol to 60% by mol relative to the total amount of the stereoisomers, and

a summed amount of the isomers (A) and (B) is 42% by mol or more relative to the total amount of the stereoisomers.

In addition, a polyimide precursor resin of the present invention is a polyimide precursor resin comprising a repeating unit (I) represented by the following general formula (4):

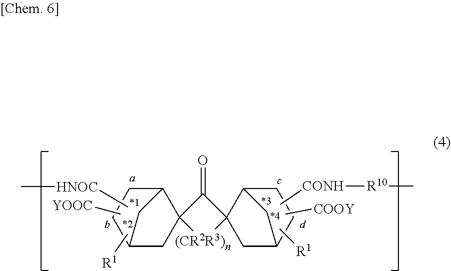

[in the formula (4), R¹, R², and R³ each independently represent one selected from the group consisting of a hydrogen atom, alkyl groups having 1 to 10 carbon atoms, and a fluorine atom, n represents an integer of 0 to 12, R¹⁰ represents an arylene group having 6 to 50 carbon atoms, Ys each independently represent one selected from the group consisting of a hydrogen atom, alkyl groups having 1 to 6 carbon atoms, and alkyl silyl groups having 3 to 9 carbon atoms, one of a bonding arm represented by *1 and a bonding arm represented by *2 is bonded to a carbon atom a forming the norbornane ring, the other of the bonding arm represented by *1 and the bonding arm represented by *2 is bonded to a carbon atom b forming the norbornane ring, one of a bonding arm represented by *3 and a bonding arm represented by *4 is bonded to a carbon atom c forming the norbornane ring, and the other of the bonding arm represented by *3 and the bonding arm represented by *4 is bonded to a carbon atom d forming the norbornane ring], wherein a content of a repeating unit (I-A) having a three-dimensional structure represented by the following general formula (5):

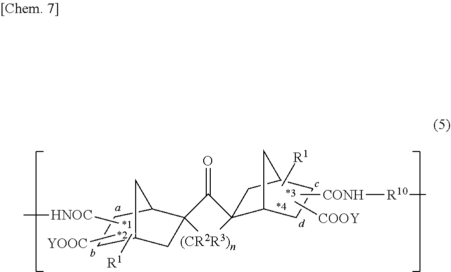

[R¹, R², R³, R¹⁰, Y, n, a to d, and *1 to *4 in the formula (5) have the same definitions as those of R¹, R², R³, R¹⁰, Y, n, a to d, and *1 to *4 in the general formula (4), respectively] is 40% by mol to 98% by mol relative to a total amount of the repeating unit (I),

a content of a repeating unit (I-B) having a three-dimensional structure represented by the following general formula (6):

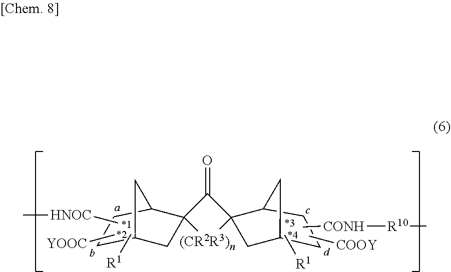

[R¹, R², R³, R¹⁰, Y, n, a to d, and *1 to *4 in the formula (6) have the same definitions as those of R¹, R², R³, R¹⁰, Y, n, a to d, and *1 to *4 in the general formula (4), respectively] is 2% by mol to 60% by mol relative to the total amount of the repeating unit (I), and

a summed amount of the repeating units (I-A) and (I-B) is 42% by mol or more relative to the total amount of the repeating unit (I).

Moreover, a polyimide of the present invention is a polyimide comprising a repeating unit (II) represented by the following general formula (7):

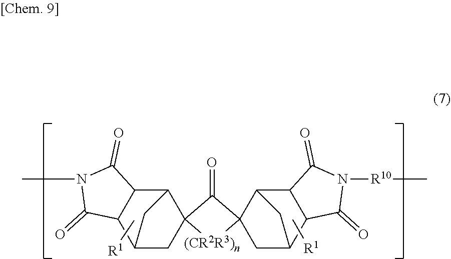

[in the formula (7), R¹, R², and R³ each independently represent one selected from the group consisting of a hydrogen atom, alkyl groups having 1 to 10 carbon atoms, and a fluorine atom, n represents an integer of 0 to 12, and R¹⁰ represents an arylene group having 6 to 50 carbon atoms], wherein

a content of a repeating unit (II-A) having a three-dimensional structure represented by the following general formula (8):

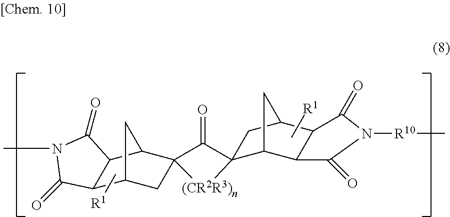

[R¹, R², R³, R¹⁰, and n in the formula (8) have the same definitions as those of R¹, R², R³, R¹⁰, and n in the general formula (7), respectively] is 40% by mol to 98% by mol relative to a total amount of the repeating unit (II),

a content of a repeating unit (II-B) having a three-dimensional structure represented by the following general formula (9):

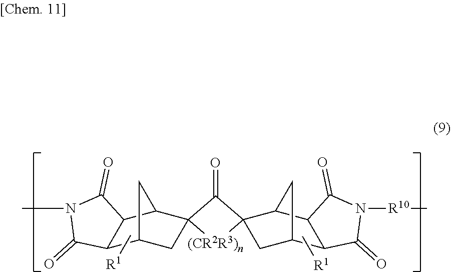

[R¹, R², R³, R¹⁰, and n in the formula (9) have the same definitions as those of R¹, R², R³, R¹⁰, and n in the general formula (7), respectively] is 2% by mol to 60% by mol relative to the total amount of the repeating unit (II), and

a summed amount of the repeating units (II-A) and (II-B) is 42% by mol or more relative to the total amount of the repeating unit (II).

In addition, a polyimide precursor resin solution of the present invention comprises the polyimide precursor resin of the present invention and an organic solvent. Moreover, a polyimide solution of the present invention comprises the polyimide of the present invention and an organic solvent. A resin solution (varnish) such as the above polyimide solution and polyimide precursor resin solution (for example, a polyamic acid solution) makes it possible to efficiently produce various forms of polyimides.

In addition, a polyimide film of the present invention is a cured product of at least one resin solution selected from the group consisting of the above polyimide precursor resin solution of the present invention and the above polyimide solution of the present invention.

### Advantageous Effects of Invention

The present invention makes it possible to provide a tetracarboxylic dianhydride that can be used as a raw material monomer for producing a polyimide that can have lower linear expansion coefficient and dielectric dissipation factor while having sufficiently high levels of heat resistance and transparency; a polyimide precursor resin that can be efficiently produced by using the tetracarboxylic dianhydride and can be used for producing a polyimide that can have lower linear expansion coefficient and dielectric dissipation factor while having sufficiently high levels of heat resistance and transparency; and a polyimide that can have lower linear expansion coefficient and dielectric dissipation factor while having sufficiently high levels of heat resistance and transparency. In addition, the present invention makes it possible to provide a polyimide precursor resin solution comprising the polyimide precursor resin; a polyimide solution comprising the polyimide; and a polyimide film that is obtained by using these solutions.

### BRIEF DESCRIPTION OF DRAWINGS

FIG. 1 is a graph of an HPLC spectrum of a product obtained in Synthesis Example 1.

FIG. 2 is a graph of a ¹H-NMR spectrum of one component (peak (A-1) component) in stereoisomers of a compound obtained in Synthesis Example 1.

FIG. 3 is a graph of a ¹³C-NMR spectrum of the one component (peak (A-1) component) in the stereoisomers of the compound obtained in Synthesis Example 1.

FIG. 4 is a graph of an HSQC spectrum of the one component (peak (A-1) component) in the stereoisomers of the compound obtained in Synthesis Example 1.

FIG. 5 is a graph of an NOESY spectrum of the one component (peak (A-1) component) in the stereoisomers of the compound obtained in Synthesis Example 1.

FIG. 6 is a graph of a ¹H-NMR spectrum of one component (peak (A-2) component) in the stereoisomers of the compound obtained in Synthesis Example 1.

FIG. 7 is a graph of a ¹³C-NMR spectrum of the one component (peak (A-2) component) in the stereoisomers of the compound obtained in Synthesis Example 1.

FIG. 8 is a graph of an HSQC spectrum of the one component (peak (A-2) component) in the stereoisomers of the compound obtained in Synthesis Example 1.

FIG. 9 is a graph of an NOESY spectrum of the one component (peak (A-2) component) in the stereoisomers of the compound obtained in Synthesis Example 1.

FIG. 10 is a graph of part of the NOESY spectrum of the one component (peak (A-1) component) in the stereoisomers of the compound obtained in Synthesis Example 1.

FIG. 11 is a graph of part of the NOESY spectrum of the one component (peak (A-2) component) in the stereoisomers of the compound obtained in Synthesis Example 1.

FIG. 12 is a graph of part of the NOESY spectrum used in analyzing a cis or trans three-dimensional configuration of two norbornane rings of the one component (peak (A-1) component) in the stereoisomers of the compound obtained in Synthesis Example 1.

FIG. 13 is a graph of part of the NOESY spectrum used in analyzing the cis or trans three-dimensional configuration of the two norbornane rings of the one component (peak (A-2) component) in the stereoisomers of the compound obtained in Synthesis Example 1.

FIG. 14 is a graph of an HPLC spectrum of a compound (crystallized white crystal) obtained in Synthesis Example 2.

FIG. 15 is a graph of an IR spectrum of the compound obtained in Example 1.

FIG. 16 is a graph of an HPLC spectrum of a compound obtained in Example 1.

FIG. 17 is a graph of an IR spectrum of a compound forming a film obtained in Example 4.

### DESCRIPTION OF EMBODIMENTS

Hereinafter, the present invention is described in detail with reference to preferred embodiments.

### [Tetracarboxylic Dianhydride]

A tetracarboxylic dianhydride of the present invention is a mixture of stereoisomers of a compound represented by the above-described general formula (1), wherein

a content of an isomer (A) represented by the above-described general formula (2) is 40% by mol to 98% by mol relative to a total amount of the stereoisomers,

a content of an isomer (B) represented by the above-described general formula (3) is 2% by mol to 60% by mol relative to the total amount of the stereoisomers, and

a summed amount of the isomers (A) and (B) is 42% by mol or more relative to the total amount of the stereoisomers.

R¹, R², and R³ in the above-described general formulae (1) to (3) are each independently one selected from the group consisting of a hydrogen atom, alkyl groups having 1 to 10 carbon atoms, and a fluorine atom, and n is an integer of 0 to 12.

The alkyl group which can be selected as any one of R¹, R², and R³ in the formulae (1) to (3) is an alkyl group having 1 to 10 carbon atoms. If the number of carbon atoms exceeds 10, the glass transition temperature is lowered, so that a sufficiently high heat resistance cannot be achieved. In addition, the number of carbon atoms of the alkyl group which can be selected as any one of R¹, R², and R³ is preferably 1 to 6, more preferably 1 to 5, further preferably 1 to 4, and particularly preferably 1 to 3, from the viewpoint that the purification is easier. In addition, the alkyl group which can be selected as any one of R¹, R², and R³ may be linear or branched. Moreover, the alkyl group is more preferably a methyl group or an ethyl group from the viewpoint of ease of purification.

R¹, R², and R³ in the formulae (1) to (3) are each independently more preferably a hydrogen atom or an alkyl group having 1 to 10 carbon atoms from the viewpoint that a higher heat resistance can be obtained in the production of a polyimide. Especially, R¹, R², and R³ are each independently more preferably a hydrogen atom, a methyl group, an ethyl group, an n-propyl group, or an isopropyl group, and particularly preferably a hydrogen atom or a methyl group, from the viewpoints that the raw materials are readily available and that the purification is easier. In addition, the multiple R¹s, R²s, and R³s in each of the formulae are particularly preferably the same, from the viewpoints of ease of purification and the like.

In addition, n in the formulae (1) to (3) represents an integer of 0 to 12. If the value of n exceeds the upper limit, the purification is difficult. In addition, an upper limit value of the numeric value range of n in the general formulae (1) to (3) is more preferably 5, and particularly preferably 3, from the viewpoints that the purification is easier and the like. In addition, a lower limit value of the numeric value range of n in the general formulae (1) to (3) is more preferably 1, and particularly preferably 2, from the viewpoint of the stability of a raw material compound. Accordingly, n in the general formulae (1) to (3) is particularly preferably an integer of 2 to 3.

In addition, the tetracarboxylic dianhydride represented by the general formula (1) may have a structure in which the two norbornane rings are arranged in cis or trans relative to the cycloalkanone ring and in which the carbonyl group (C═O) in the cycloalkanone ring may have an exo or endo three-dimensional configuration relative to each of the two norbornane rings. Here, as is clear from the structure represented by the general formula (2), the isomer (A) is a so-called trans-exo-exo isomer in which the two norbornane rings are arranged in trans relative to the cycloalkanone ring (a trans isomer of a spiro-fused ring) and the carbonyl group (C═O) in the cycloalkanone ring has an exo three-dimensional configuration relative to each of the two norbornane rings. On the other hand, the isomer (B) is a so-called cis-exo-exo isomer in which the two norbornane rings are arranged in cis relative to the cycloalkanone ring (a cis isomer of a spiro-fused ring) and the carbonyl group (C═O) in the cycloalkanone ring has an exo three-dimensional configuration relative to each of the two norbornane rings.

In the Specification, regarding three-dimensional structures in isomers and repeating units, the expressions “trans-exo-exo, cis-exo-exo, trans-endo-endo, cis-endo-endo, trans-exo-endo, cis-exo-endo” express the relations between two norbornane rings (for a compound represented by a general formula (iv) described later, however, norbornene rings) and a cycloalkanone ring. In order to further describe the relations between three-dimensional structures of two norbornane rings and a cycloalkanone ring and the expressions of three-dimensional structures such as trans-exo-exo and cis-exo-exo, the relations between the three-dimensional structures at the portions of two norbornane rings and a cycloalkanone ring and the expressions are represented by the following formulae (S1) to (56):

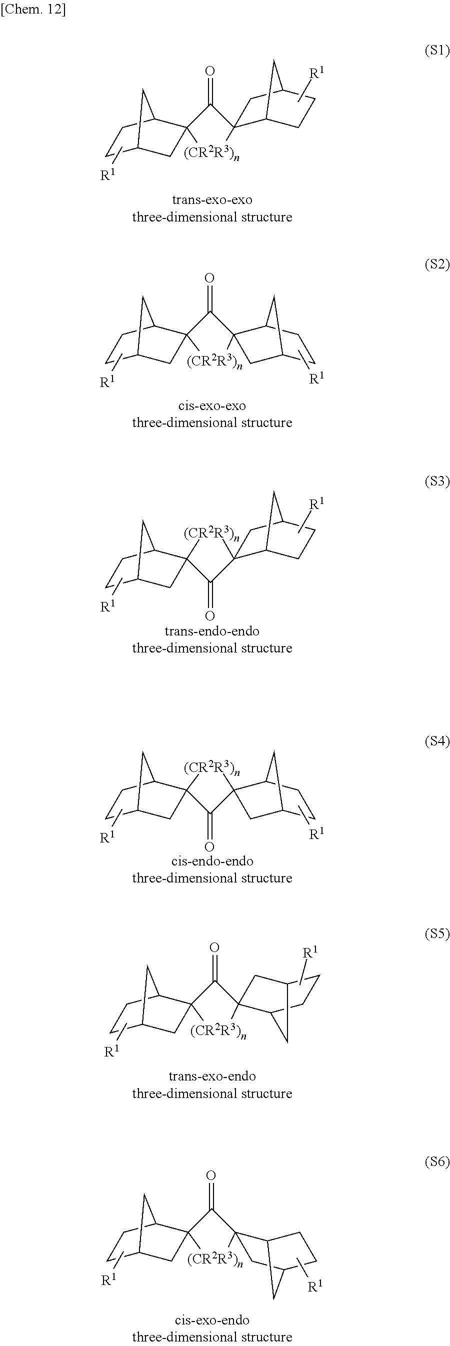

As above, the compound represented by the general formula (1) is such that the portion of the two norbornane rings and the cycloalkanone ring in the compound can have any of 6 three-dimensional structures as described in the formulae (51) to (S6). Specifically, the tetracarboxylic dianhydride represented by the general formula (1) can contain, based on the three-dimensional configuration of the two norbornane rings and the cycloalkanone ring in the compound, any of 6 stereoisomers, that is, an isomer having a trans-exo-exo three-dimensional structure represented by the formula (Si) (a trans-exo-exo isomer: corresponding to the isomer (A) represented by the general formula (2)); an isomer having a cis-exo-exo three-dimensional structure represented by the formula (S2) (a cis-exo-exo isomer: corresponding to the isomer (B) represented by the general formula (3)); an isomer having a trans-endo-endo three-dimensional structure represented by the formula (S3) (a trans-endo-endo isomer); an isomer having a cis-endo-endo three-dimensional structure represented by the formula (S4) (a cis-endo-endo isomer); an isomer having a trans-exo-endo three-dimensional structure represented by the formula (S5) (a trans-exo-endo isomer); and an isomer having a cis-exo-endo three-dimensional structure represented by the formula (S6) (a cis-exo-endo isomer).

Note that the content ratios of the isomers in such tetracarboxylic dianhydride can be calculated by obtaining an area ratio of a peak based on each isomer, based on a graph of a spectrum, which is obtained by a high-performance liquid chromatography (HPLC) measurement, and using a calibration curve, for example. Such HPLC measurement can be conducted by using the trade name “1200 Series” manufactured by Agilent Technologies as a measurement device, using the trade name “ZORBAX SB-CN (particle size: 5 μm, diameter 4.6 mm, and length 250 mm)” manufactured by Agilent Technologies as a column, using a mixture of n-hexane and 1,4-dioxane (n-hexane/1,4-dioxane=40 mL/60 mL) as a solvent, setting the flow speed of the solvent to 1.0 mL/min., setting the detection wavelength of a diode array detector (DAD) to 230 nm, setting the temperature to 35° C., and preparing a sample obtained by adding 1 mg of tetracarboxylic dianhydride (measurement target) to 1.5 mL of a solvent. In addition, the calibration curve can be obtained by obtaining a HPLC spectrum under the measurement condition using dicyclopentadiene or naphthalene as a reference sample. Note that in such measurement, the relation between the three-dimensional structure of tetracarboxylic dianhydride and the peak position of the HPLC spectrum is obtained as described below.

Specifically, first, separately from the above-described HPLC measurement, a norbornene compound (a compound represented by a general formula (iv) described later: for example, 5-norbornene-2-spiro-2′-cyclopentanone-5′-spiro-2″-5″-norbornene), which serves as a raw material of tetracarboxylic dianhydride, is prepared for each isomer, and esterification and conversion to an acid dianhydride are conducted for each isomer to prepare authentic samples of acid dianhydride (6 authentic samples: an authentic sample of a trans-exo-exo isomer, an authentic sample of a cis-exo-exo isomer, an authentic sample of a trans-endo-endo isomer, an authentic sample of a cis-endo-endo isomer, an authentic sample of a trans-exo-endo isomer, and an authentic sample of a cis-exo-endo isomer) for each isomer. Then, HPLC measurement is conducted on the authentic samples of acid dianhydride for each isomer (note that the three-dimensional structure of the isomer of the raw material basically does not change and is maintained in the esterification and the conversion to an acid dianhydride) by employing the same conditions as those employed in the HPLC measurement on tetracarboxylic dianhydride, which is the measurement target, to obtain the peak position of the HPLC graph for each authentic sample. Then the peak position of each of the HPLC spectrum of the authentic sample of the trans-exo-exo isomer, the HPLC spectrum of the authentic sample of the cis-exo-exo isomer, the HPLC spectrum of the authentic sample of the trans-endo-endo isomer, the HPLC spectrum of the authentic sample of the cis-endo-endo isomer, the HPLC spectrum of the authentic sample of the trans-exo-endo isomer, and the HPLC spectrum of the authentic sample of the cis-exo-endo isomer is compared with the peak position of the graph of the HPLC spectrum of tetracarboxylic dianhydride which is the measurement target, the peak at the same peak position is determined as the peak of the isomer having the same three-dimensional structure as the authentic sample, and the ratio of each isomer is obtained. In addition, the area ratio of a peak based on each isomer can be directly obtained using the above-described measurement device in the graph of the HPLC spectrum. Note that in such HPLC measurement, peaks basically appear during a retention time of approximately 2.5 minutes to 4.5 minutes, the peak near a retention time of 3.94 minutes is a peak derived from the isomer having the trans-exo-exo three-dimensional structure, the peak near a retention time of 3.01 minutes is a peak derived from the isomer having the cis-exo-exo three-dimensional structure, the peak near a retention time of 3.35 minutes is a peak derived from the isomer having the cis-exo-endo three-dimensional structure, the peak near a retention time of 3.70 minutes is a peak derived from the isomer having the trans-exo-endo three-dimensional structure, the peak near a retention time of 4.08 minutes is a peak derived from the trans-endo-endo isomer, and the peak near a retention time of 3.08 minutes is a peak derived from the cis-endo-endo isomer. Note that although there is a slight deviation depending on the column lot and the like, peaks appear substantially at the positions of the above retention times. In this way, the method for measuring a content ratio of each isomer is a method including: comparing the peak position of the HPLC measurement of the measurement target with the peak position of each of the aforementioned authentic samples; obtaining the type of the isomer at the peak position, and obtaining the content ratio of each isomer by using the area ratio of the peak.

In addition, the tetracarboxylic dianhydride of the present invention is a mixture of the stereoisomers of the compound represented by the general formula (1) as described above (that can contain the above-described 6 stereoisomers). In the tetracarboxylic dianhydride of the present invention, the content of the isomer (A) represented by the general formula (2) is 40% by mol to 98% by mol (more preferably 45% by mol to 98% by mol, further preferably 65% by mol to 97% by mol, particularly preferably 75% by mol to 96% by mol, and most preferably 80% by mol to 96% by mol) relative to the total amount of the stereoisomers contained in the mixture (the summed amount of all the stereoisomers contained in the tetracarboxylic dianhydride). If the content of the isomer (A) is less than the lower limit, it is difficult to obtain lower values of the linear expansion coefficient and the dielectric dissipation factor of the polyimide when the polyimide is produced using the tetracarboxylic dianhydride. On the other hand, if the content of the isomer (A) exceeds the upper limit, a resin produced using the tetracarboxylic dianhydride tends to be brittle.

In addition, in the tetracarboxylic dianhydride of the present invention, the content of the isomer (B) represented by the general formula (3) is 2% by mol to 60% by mol (more preferably 2 to 55% by mol, further preferably 3% by mol to 35% by mol, particularly preferably 4% by mol to 25% by mol, and most preferably 4% by mol to 20% by mol) relative to the total amount of the stereoisomers contained in the mixture (summed amount of all the stereoisomers contained in the tetracarboxylic dianhydride). If the content of the isomer (B) is less than the lower limit, it is difficult to obtain lower values of the linear expansion coefficient and the dielectric dissipation factor of the polyimide when the polyimide is produced using the tetracarboxylic dianhydride. On the other hand, if the content of the isomer (B) exceeds the upper limit, a resin produced using the tetracarboxylic dianhydride tends to be brittle.

Moreover, in the tetracarboxylic dianhydride of the present invention, the summed amount of the isomers (A) and (B) is 42% by mol or more (more preferably 50% by mol or more, further preferably 74% by mol or more, particularly preferably 85% by mol or more, and most preferably 90% by mol or more) relative to the total amount of the stereoisomers contained in the mixture (summed amount of all the stereoisomers contained in the tetracarboxylic dianhydride). If the summed amount of the isomers (A) and (B) is less than the lower limit, it is difficult to obtain lower values of the linear expansion coefficient and the dielectric dissipation factor of the polyimide when the polyimide is produced using the tetracarboxylic dianhydride.

Moreover, if the tetracarboxylic dianhydride of the present invention contains stereoisomers other than the isomers (A) and (B), the summed amount of the trans-endo-endo isomer and the cis-endo-endo isomer is preferably 10% by mol or less and more preferably 0 to 5% by mol relative to the total amount of the isomers (the summed amount of all the isomers). Furthermore, if the tetracarboxylic dianhydride of the present invention contains stereoisomers other than the isomers (A) and (B), the summed amount of the trans-exo-endo isomer and the cis-exo-endo isomer is preferably 30% by mol or less and more preferably 0 to 20% by mol relative to the total amount of the isomers (the summed amount of all the isomers). If the summed amount of the other isomers exceeds the upper limit, it tends to be difficult to obtain lower values of the linear expansion coefficient and the dielectric dissipation factor of the polyimide when the polyimide is produced using the tetracarboxylic dianhydride.

As the method for producing the tetracarboxylic dianhydride of the present invention, a method for producing a tetracarboxylic dianhydride including: producing a compound represented by a general formula (iv) by using a reaction expressed by the following reaction formula:

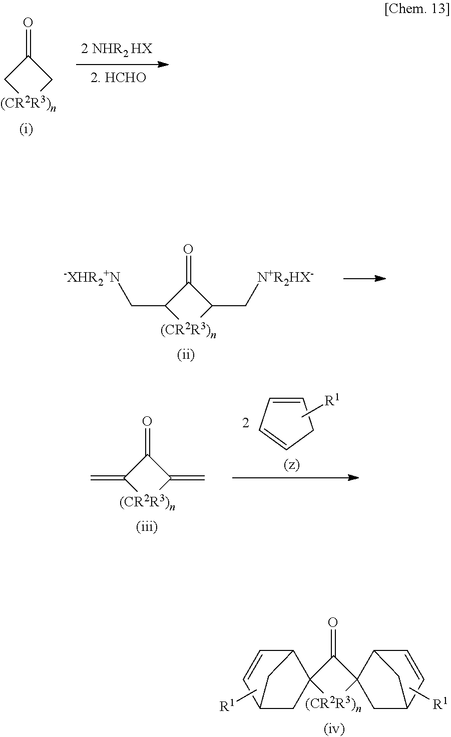

[R¹, R², R³, and n in the formula have the same definitions as those of R¹, R², R³, and n in the general formula (1), respectively, Rs each independently represent a monovalent organic group (for example, a linear saturated hydrocarbon group having 1 to 20 carbon atoms or the like) that can form an amine, and X⁻ represents a monovalent ion (for example, a halogen ion, a hydrogen sulfate ion, an acetic acid ion, or the like) that can form an ammonium salt with the amine]; thereafter forming a tetracarboxylic ester by reacting the compound represented by the general formula (iv) with an alcohol and carbon monoxide; and then converting the tetracarboxylic ester to an acid dianhydride.

The reaction expressed by the above reaction formula is a reaction in which after a Mannich base represented by the general formula (ii) is obtained from a cycloalkanone represented by the general formula (i), formaldehyde, and an ammonium salt of a secondary amine (a compound represented by the formula: NHR₂HX, for example, hydrochloride, sulfate, acetate, or the like), an amine compound is removed from the Mannich base to form a divinyl ketone represented by the general formula (iii), and a compound represented by the general formula (iv) is prepared by reacting the divinyl ketone with a cyclopentadiene which may have a substituent R¹ (a cyclopentadiene represented by the general formula (z)). Here, the method for forming a Mannich base, the method for removing an amine compound from the Mannich base to form a divinyl ketone represented by the general formula (iii), and the like are not particularly limited, and known methods and conditions and the like (for example, conditions, raw material compounds, and the like employed in the method explained in paragraphs [0071] to paragraph [0100] of WO 2011/099518) may be used as appropriate with no particular limitations. Note that as the temperature condition in reacting a cycloalkanone represented by the general formula (i), formaldehyde, and an ammonium salt of a secondary amine (the temperature condition in synthesizing a Mannich base), a relatively low-temperature condition of 5 to 25° C. (more preferably, 10 to 25° C.) may be employed. In addition, as a cycloalkanone represented by the general formula (i), formaldehyde, and a compound represented by the formula: NHR₂HX (an ammonium salt of a secondary amine), known ones (for example, those described in paragraph [0080] of WO 2011/099518, and the like) may be used as appropriate.

Here, if a divinyl ketone represented by the general formula (iii) and a cyclopentadiene represented by the general formula (z) are reacted under a normal temperature condition as conventionally employed (Examples of PTL 1 employed a temperature of approximately 120° C.), the reaction is a so-called Diels-Alder reaction, which chemical-kinetically advantageously form endo adducts. Hence, the compound represented by the general formula (iv) may contain an isomer having a trans-exo-exo three-dimensional structure (a trans-exo-exo isomer), an isomer having a cis-exo-exo three-dimensional structure (a cis-exo-exo isomer), an isomer having a trans-exo-endo three-dimensional structure (a trans-exo-endoisomer), an isomer having a cis-exo-endo three-dimensional structure (a cis-exo-endo isomer), an isomer having a trans-endo-endo three-dimensional structure (a trans-endo-endo isomer), and an isomer having a cis-endo-endo three-dimensional structure (a cis-endo-endo isomer) as stereoisomers of the compound. Among these, chemical-kinetically advantageous endo adducts were preferentially produced while thermodynamically stable exo adducts were hardly produced, so that the content of the exo adducts was less than 1% and it was impossible to isolate these (note that the three-dimensional structures such as “cis-exo-exo” and “trans-endo-endo” mentioned herein refer to three-dimensional structures in the case where both of the norbornane rings in the three-dimensional structures represented by the formulae (Si) to (S6) are replaced with norbornene rings with the crosslinkage on the side where the number of carbon atoms is large (the side of two carbon atoms) and the crosslinkage on the side where the number of carbon atoms is small (the side of one carbon atom) being directed on the same side).

From such a viewpoint, in order to produce the tetracarboxylic dianhydride of the present invention, it is necessary to employ a specific temperature condition in reacting a divinyl ketone with a cyclopentadiene represented by the general formula (X), and the temperature condition during the reaction should be a high-temperature condition of 140° C. to 300° C. (more preferably 150° C. to 250° C.) in order to further increase the content of the isomer having a trans-exo-exo three-dimensional structure (the trans-exo-exo isomer) and the isomer having a cis-exo-exo three-dimensional structure (the cis-exo-exo isomer). If such a temperature condition is less than the lower limit, the ratio of the endo adducts tends to increase. On the other hand, if the temperature condition exceeds the upper limit, polymers derived from the cyclopentadiene tend to increase to lower the yield. By employing such a high-temperature condition for the reaction, it is possible to prepare a mixture of stereoisomers of the compound represented by the general formula (iv) in which the summed amount of the isomer having a trans-exo-exo three-dimensional structure and the isomer having a cis-exo-exo three-dimensional structure is at sufficiently high concentration (preferably 42% by mol or more, further preferably 60% by mol or more, and particularly preferably 80% by mol or more). Note that as the conditions other than the temperature condition (the atmosphere condition, the pressure condition, the solvent condition, the type of a cyclopentadiene represented by the general formula (z), and the like), conditions that can be employed (used) in the known Diels-Alder reaction may be employed as appropriate, and for example, conditions that are employed in the method described in paragraphs [0071] to paragraph [0100] of WO 2011/099518 can be employed. As described above, as the method described in the above reaction formula, the same method as the known method (for example, the method described in paragraphs [0071] to [0100] of WO 2011/099518) can be basically employed except that the temperature condition in reacting a divinyl ketone represented by the general formula (iii) and a cyclopentadiene represented by the general formula (z) is set to the above-described specific temperature condition (high-temperature condition).

As described above, by employing the high-temperature condition and reacting a divinyl ketone represented by the general formula (iii) and a cyclopentadiene represented by the general formula (z), it is possible to prepare a mixture of stereoisomers of a compound represented by the general formula (iv) in which a content of an isomer having a trans-exo-exo three-dimensional structure (a trans-exo-exo isomer) is 40% by mol or more relative to a total amount of the isomers (a summed amount of all the isomers), a content of an isomer having a cis-exo-exo three-dimensional structure (a cis-exo-exo isomer) is 2% by mol or more relative to the total amount of the isomers (the summed amount of all the isomers), and a summed amount of the isomer having the trans-exo-exo three-dimensional structure and the isomer having a cis-exo-exo three-dimensional structure is 42% by mol or more relative to the total amount of the isomers (summed amount of all the isomers).

Note that the three-dimensional structure of each stereoisomer in a compound represented by the general formula (iv) can be identified, for example, by measuring the one-dimensional NMR (¹H and ¹³C) and the two-dimensional NMR (COSY, HSQC, HMBC, and NOESY), and the like. In addition, the concentration (content ratio) of each stereoisomer in a compound represented by the general formula (iv) can be obtained by conducting HPLC measurement and then based on the peak area ratio. Note that in such HPLC measurement, peaks appear during a retention time of approximately 3 minutes to 15 minutes, the peak near a retention time of 10.5 minutes is a peak derived from the isomer having a trans-exo-exo three-dimensional structure and the isomer having a cis-exo-exo three-dimensional structure, the peak near a retention time of 8.0 minutes is a peak derived from the isomer having a cis-exo-endo three-dimensional structure and the isomer having a trans-exo-endo three-dimensional structure, and the peak near a retention time of 6.4 minutes is a peak derived from the trans-endo-endo isomer and the cis-endo-endo isomer. Note that as such HPLC measurement of a compound represented by the general formula (iv), it is preferable to employ the same method as the method employed in checking content ratios of stereoisomers in a compound obtained in Synthesis Example 1, which will be described later. In addition, the three-dimensional structure of the isomer at each peak position can be measured by the one-dimensional NMR (1H and ¹³C) and the two-dimensional NMR (COSY, HSQC, HMBC, and NOESY) as described above.

In addition, the method for forming a tetracarboxylic ester by reacting the compound represented by the general formula (iv) with an alcohol and carbon monoxide (esterification method) is not particularly limited, and a known method that is capable of introducing an ester group to a carbon atom forming a double bond (a known method that is capable of alkoxy carbonylation) may be employed as appropriate, and for example, a method described in International Publication No. WO2014/050810, a method described in Japanese Unexamined Patent Application Publication No. 2015-137231, a method describe in Japanese Unexamined Patent Application Publication No. 2014-218460, methods described in International Publication No. WO2011/099517 and WO 2011/099518, and the like may be used as appropriate. Note that as an alcohol and the like used in such esterification as well, known ones may be used as appropriate, and for example, those used in the method described in WO 2014/050810, the method described in JP 2015-137231, and the method described in JP 2014-218460, the methods described in WO 2011/099517 and WO 2011/099518, and the like may be used as appropriate.

In addition, in such esterification, when an alcohol represented by a formula: R²⁰OH [in the formula, R²⁰s each independently represent an alkyl group having 1 to 10 carbon atoms or the like] is used as an alcohol, it is possible to obtain a tetracarboxylic ester represented by the following general formula (v):

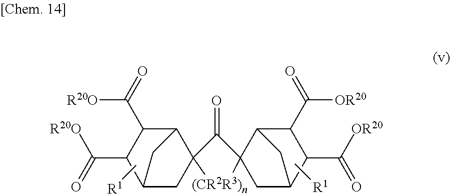

[R¹, R², R³, and n in the formula (v) have the same definition as those of R¹, R², R₃, and n in the general formulae (1) to (3), and R²⁰s each independently represent an alkyl group having 1 to 10 carbon atoms or the like](note that the norbornane ring and the cyclopentadiene ring in the formula (v) can have three-dimensional structures represented by the aforementioned formulae (S1) to (S6)). Note that in such esterification, the three-dimensional structure of the norbornene ring in the compound represented by the general formula (iv), which is used as a raw material, is sufficiently maintained, and it is basically possible to form a tetracarboxylic ester while maintaining the three-dimensional structures of isomers in the compound represented by the general formula (iv) (note that J. Am. Chem. Soc., vol. 98, p. 1810, 1976 reported that in esterification, a methoxycarbonyl group is introduced to a bridgehead position (methylene head) in exo configuration while the three-dimensional structure of a raw material compound (norbornene) is maintained, and from such report, it is obvious that the three-dimensional structure of a norbornene ring is maintained in esterification). Hence, such esterification makes it possible to also form a carboxylic ester in which a content of an isomer having a trans-exo-exo three-dimensional structure (a trans-exo-exo isomer) is 40% by mol or more relative to a total amount of the isomers, a content of an isomer having a cis-exo-exo three-dimensional structure (a cis-exo-exo isomer) is 2% by mol or more relative to the total amount of the isomers, and a summed amount of the isomer having a trans-exo-exo three-dimensional structure and the isomer having a cis-exo-exo three-dimensional structure is 42% by mol or more relative to the total amount of the isomers.

Here, in such esterification, it is preferable to, after obtaining a crude product by reacting a compound represented by the general formula (iv) with an alcohol and carbon monoxide, dissolve the crude product in an organic solvent (for example, toluene, xylene, ethyl acetate, acetonitrile, acetic acid, ethanol, or the like) to prepare a solution, concentrate the solution to precipitate a crystal, and collect the crystal through filtration to obtain a carboxylic ester (obtain a carboxylic ester through crystallization). In a case where the esterification is conducted after a compound represented by the general formula (iv) is prepared, the isomer having a trans-exo-exo three-dimensional structure and the isomer having a cis-exo-exo three-dimensional structure which basically have low solubility to an organic solvent precipitate as precipitates (crystal) by crystallization, and the other components basically remain on the filtrate side. Hence it is possible to achieve a higher concentration of the trans-exo-exo isomer and the cis-exo-exo isomer in the tetracarboxylic ester (tetraester) obtained after the crystallization (for example, it is also possible to allow only the trans-exo-exo isomer and the cis-exo-exo isomer to be contained). Hence, by executing such a crystallization step, it is also possible to more efficiently produce the carboxylic ester in which the content of the trans-exo-exo isomer is 40% by mol or more relative to the total amount of the isomers, the content of the cis-exo-exo isomer is 2% by mol or more relative to the total amount of the isomers, and a summed amount of the trans-exo-exo isomer and the cis-exo-exo isomer is 42% by mol or more relative to the total amount of the isomers.

Note that the concentration (content ratio) of each stereoisomer in the tetracarboxylic ester (tetraester) can be obtained by conducting HPLC measurement and then based on the peak area ratio. In such HPLC measurement of the tetracarboxylic ester (tetraester), it is preferable to employ the same method as the method employed in checking content ratios of stereoisomers in the compound obtained in Synthesis Example 1, which will be described later (which is the same method as a method employed in checking content ratios of stereoisomers in a compound obtained in Synthesis Example 2, which will be described later. In addition, in such measurement, the relation between the three-dimensional structures of the tetracarboxylic ester (tetraester) and the peak positions of the HPLC spectrum is obtained as described below. Specifically, first, besides the HPLC measurement, a norbornene compound (a compound represented by the general formula (iv): for example, 5-norbornene-2-spiro-2′-cyclopentanone-5′-spiro-2″-5″-norbornene), which serves as a raw material of the tetracarboxylic dianhydride, is separated for each isomer, and esterification is conducted for each isomer to prepare an authentic sample of a tetracarboxylic ester for each isomer (6 authentic samples: an authentic sample of the trans-exo-exo isomer, an authentic sample of the cis-exo-exo isomer, an authentic sample of the trans-endo-endo isomer, an authentic sample of the cis-endo-endo isomer, an authentic sample of the trans-exo-endo isomer, and an authentic sample of the cis-exo-endo isomer). Then, HPLC measurement is conducted on the authentic sample of tetracarboxylic ester of each isomer under the same conditions as the conditions employed in the HPLC measurement of the tetracarboxylic ester to be the measurement target to obtain the peak position of the HPLC graph for each authentic sample. Then, the peak position in the graph (each graph) of the HPLC spectrum of each authentic sample is compared with the peak position of the graph of the HPLC spectrum of the tetracarboxylic ester to be the measurement target to identify that the peak at the same peak position is the peak of the isomer having the same three-dimensional structure as that of the authentic sample and obtain the ratio of each isomer. In addition, in such HPLC measurement, peaks appear during a retention time of approximately 2.0 minutes to 2.9 minutes, the peak near a retention time of 2.5 minutes is a peak derived from the isomer having the trans-exo-exo three-dimensional structure and the isomer having the cis-exo-exo three-dimensional structure, the peak near a retention time of 2.1 minutes is a peak derived from the isomer having the cis-exo-endo three-dimensional structure and the isomer having the trans-exo-endo three-dimensional structure, and the peak near a retention time of 2.3 minutes is a peak derived from the trans-endo-endo isomer and the cis-endo-endo isomer. Note that although there is a slight deviation depending on the column lot and the like, peaks appear substantially at the positions of the above retention times, and accordingly the content ratios can be obtained by using the area ratios of the peaks.

In addition, the method for converting the tetracarboxylic ester (tetraester) to an acid dianhydride (the method for conversion to an acid anhydride) is not particularly limited, and any known method that is capable of converting the tetraester to an acid dianhydride to obtain tetracarboxylic dianhydride may be employed as appropriate, and for example, a method including heating a tetraester in a carboxylic acid having 1 to 5 carbon atoms, and the like may be employed as appropriate. As such a method for converting a tetraester to an acid dianhydride, for example, methods and conditions employed in the method described in WO 2014/050788, the method described in WO 2015/178261, the method described in WO 2011/099518, the method described in JP 2015-218160, and the like may be employed as appropriate (also for various conditions and the like including a carboxylic acid to be used, and a catalyst, the methods employed in the above-described known methods may be used as appropriate).

Note that in such conversion to an acid dianhydride, the three-dimensional structures of the norbornane rings are sufficiently maintained, and it is basically possible to produce a tetracarboxylic dianhydride while maintaining the three-dimensional structures of the isomers in the tetracarboxylic ester and the content ratios of the isomers. For example, from the description in page 1117 of Macromolecules (vol. 27) published on 1994, it was revealed that the three-dimensional configuration of a raw material compound is maintained in a transesterification reaction using an acid catalyst. In a case where conversion to an acid dianhydride is conducted by a transesterification reaction using an acid catalyst, it basically is possible to produce a tetracarboxylic dianhydride while maintaining the three-dimensional structures of the isomers in a tetracarboxylic ester used as a raw material and the content ratios of the isomers. Hence, the method as described above makes it possible to efficiently produce the tetracarboxylic dianhydride of the present invention.

Note that in a case where the conversion to an acid anhydride is conducted using a heterogeneous catalyst by employing the method as described in WO 2014/050788, it is also possible to prepare products having different ratios of the content of a trans-exo-exo isomer (a trans-exo-exo isomer) and the content of a cis-exo-exo isomer (a cis-exo-exo isomer) by separately collecting a reaction product remaining in a solution and a reaction product attached to the heterogeneous catalyst (because the ratios of the isomers can vary depending on portions to be collected).

### [Polyimide Precursor Resin]

The polyimide precursor resin of the present invention is a polyimide precursor resin comprising a repeating unit (I) represented by the general formula (4), wherein

a content of a repeating unit (I-A) having a three-dimensional structure represented by the general formula (5) is 40% by mol to 98% by mol relative to a total amount of the repeating unit (I), a content of a repeating unit (I-B) having a three-dimensional structure represented by the general formula (6) is 2% by mol to 60% by mol relative to the total amount of the repeating unit (I), and a summed amount of the repeating units (I-A) and (I-B) is 42% by mol or more relative to the total amount of the repeating unit (I).

R¹, R², R³, and n in such general formulae (4) to (6) have the same definitions as those of R¹, R², R³, and n in the general formulae (1) to (3), respectively (the preferred ones and preferred conditions also have the same definitions).

In addition, the arylene group which can be selected as R¹⁰ in the general formulae (4) to (6) is an arylene group having 6 to 50 carbon atoms. The number of carbon atoms of the arylene group is preferably 6 to 40, more preferably 6 to 30, and further preferably 12 to 20. If the number of carbon atoms is less than the lower limit, the heat resistance of the polyimide obtained by imidization tends to decrease. On the other hand, if the number of carbon atoms exceeds the upper limit, the moldability to various forms (for example, a film and the like) tends to decrease.

In addition, the arylene group which can be selected as R¹⁰ in the general formulae (5) and (6) is preferably at least one of groups represented by the following general formulae (15) to (19):

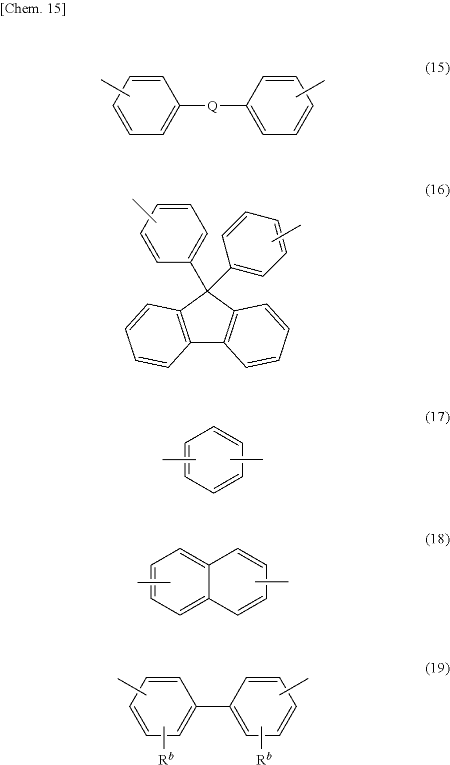

[Q in the formula (15) represents one selected from the group consisting of groups represented by formulae: —C₆H₄—, —CONH—C₆H₄—NHCO—, —NHCO—C₆H₄—CONH—, —O—C₆H₄—CO—C₆H₄—O—, —OCO—C₆H₄—COO—, —OCO—C₆H₄—C₆H₄—COO—, —OCO—, —NC₆Hs—, —CO—NC₄H₈N—CO—, —C₁₃H₁₀—, —(CH₂)₅—, —O—, —S—, —CO—, —CONH—, —NHCO—, —SO₂—, —C(CF₃)₂—, —C(CH₃)₂—, —CH₂—, —(CH₂)₂—, —(CH₂)₃—, — (CH₂)₄, —(CH₂)₅—, —O—C₆H₄—C(CH₃)₂—C₆H₄—O—, —O—C₆H₄—C(CF₃)₂—C₆H₄—O—, —O—C₆H₄—SO₂—C₆H₄—O—, —C(CH₃)₂—C₆H₄—C(CH₃)₂—, —O—C₆H₄—C₆H₄—O—, and —O—C₆H₄—O—, and R^{b} in the formula (19) represents one selected from the group consisting of a hydrogen atom, a fluorine atom, a methyl group, an ethyl group, and a trifluoromethyl group].

In addition, from the viewpoint that a cured product having a heat resistance, a transparency, and a mechanical strength at sufficient levels in a more-balanced manner can be obtained, the arylene group which can be selected as R¹⁰ in the general formulae (4) to (6) is preferably a divalent group (an arylene group) formed by removing two amino groups from at least one aromatic diamine selected from the group consisting of 4,4′-diaminobenzanilide (abbreviated name: DABAN), 4,4′-diaminodiphenyl ether (abbreviated name: DDE), 3,4′-diaminodiphenyl ether (abbreviated name: 3,4-DDE), 2,2′-bis(trifluoromethyl)benzidine (abbreviated name: TFMB), 9,9′-bis(4-aminophenyl)fluorene (abbreviated name: FDA), p-diaminobenzene (abbreviated name: PPD), 2,2′-dimethyl-4,4′-diaminobiphenyl (abbreviated name: m-tol), 3,3′-dimethyl-4,4′-diaminobiphenyl (alias: o-tolidine), 4,4′-diphenyl diamino methane (abbreviated name: DDM), 4-aminophenyl-4-aminobenzoic acid (abbreviated name: BAAB), 4,4′-bis(4-aminobenzamide)-3,3′-dihydroxybiphenyl (abbreviated name: BABB), 3,3′-diaminodiphenyl sulfone (abbreviated name: 3,3′-DDS), 1,3-bis(3-aminophenoxy)benzene (abbreviated name: APB-N), 1,3-bis(4-aminophenoxy)benzene (abbreviated name: TPE-R), 1,4-bis(4-aminophenoxy)benzene (abbreviated name: TPE-Q), 4,4′-bis(4-aminophenoxy)biphenyl (abbreviated name: 4-APBP), 4,4″-diamino-p-terphenyl, bis[4-(4-aminophenoxy)phenyl]sulfone (abbreviated name: BAPS), bis[4-(3-aminophenoxy)phenyl]sulfone (abbreviated name: BAPS-M), 2,2′-bis[4-(4-aminophenoxy)phenyl]propane (abbreviated name: BAPP), 2,2-bis[4-(4-aminophenoxy)phenyl]hexafluoropropane (abbreviated name: HFBAPP), bis[4-(4-aminophenoxy)phenyl]ketone (abbreviated name: BAPK), 4,4′-diaminodiphenyl sulfone (abbreviated name: 4,4′-DDS), (2-phenyl-4-aminophenyl)-4-aminobenzoate (4-PHBAAB), 4,4″-diamino-p-terphenyl (abbreviated name: Terphenyl), bis(4-aminophenyl) sulfide (abbreviated name: ASD), bisaniline M, bisaniline P, 2,2′″-diamino-p-quaterphenyl 2,3′″-diamino-p-quaterphenyl, 2,4′″-diamino-p-quaterphenyl, 3,3′″-diamino-p-quaterphenyl, 3,4′″-diamino-p-quaterphenyl, 4,4′″-diamino-p-quaterphenyl, 2,6-diaminonaphthalene, 1,5-diaminonaphthalene, and 1,4-diaminonaphthalene.

Note that the polyimide precursor resin of the present invention may contain two or more repeating units having different R¹⁰s in the general formulae (4) to (6). In the case where the polyimide precursor resin contains such two or more repeating units having different R¹⁰s, the combination of R¹⁰s is preferably a combination of a divalent group formed by removing two amino groups from at least one compound selected from the group consisting of DABAN, PPD, TFMB, m-Tol, o-tolidine, Terphenyl, and FDA (hereinafter, the compound is sometime simply referred to as an “aromatic diamine (A)”) and a divalent group formed by removing two amino groups from at least one compound selected from the group consisting of DDE, TPE-Q, TPE-R, APB-N, 4-APBP, BAPP, HFBAPP, bisaniline M, bisaniline P, BAAB, and 4-PHBAAB (hereinafter, the compound is sometime simply referred to as an “aromatic diamine (B)”) from the viewpoint of expressing properties such as heat resistance, transparency, mechanical strength, linear expansion coefficient, permittivity, and dielectric dissipation factor in a more-balanced manner. In the case where the polyimide precursor resin contains two or more repeating units having different R¹⁰s as described above, the aromatic diamine (A) is more preferably at least one selected from the group consisting of DABAN, PPD, TFMB, m-Tol, o-tolidine, Terphenyl, and FDA (further preferably DABAN, PPD, TFMB, and FDA) and the aromatic diamine (B) is more preferably at least one selected from the group consisting of DDE, TPE-Q, TPE-R, APB-N, 4-APBP, bisaniline M, bisaniline P, BAAB, and 4-PHBAAB (further preferably 4-APBP and bisaniline M) from the viewpoint of heat resistance, transparency, mechanical strength, linear expansion coefficient, permittivity, and dielectric dissipation factor. Moreover, it is possible to use a mixture of multiple compounds as one of the aromatic diamine (A) and the aromatic diamine (B) and one compound as the other in combination, or it is possible to use mixtures of multiple compounds respectively as both of the aromatic diamine (A) and the aromatic diamine (B) in combination, or further it is possible to use single compounds respectively as the aromatic diamine (A) and the aromatic diamine (B) in combination. In this way, multiple compounds may be selected from the aromatic diamine (A) and the aromatic diamine (B) and used in combination in accordance with the application and the like.

Ys in the general formulae (4) to (6) each independently represent one selected from the group consisting of a hydrogen atom, alkyl groups having 1 to 6 carbon atoms (preferably 1 to 3 carbon atoms), and alkyl silyl groups having 3 to 9 carbon atoms. Regarding Ys, the type of the substituent and the introduction rate of the substituent may be changed by changing the production conditions thereof as appropriate. When Ys are both hydrogen atoms (in the case of a repeating unit of polyamic acid), the production of a polyimide tends to be easy.

In addition, when Y in the general formulae (4) to (6) is an alkyl group having 1 to 6 carbon atoms (preferably 1 to 3 carbon atoms), the storage stability of the polyimide precursor resin tends to be better. In addition, when Y is an alkyl group having 1 to 6 carbon atoms (preferably 1 to 3 carbon atoms), Y is more preferably a methyl group or an ethyl group. In addition, when Y in the general formulae (4) to (6) is an alkyl silyl group having 3 to 9 carbon atoms, the solubility of the polyimide precursor resin tends to be better. When Y is an alkyl silyl group having 3 to 9 carbon atoms, Y is more preferably a trimethylsilyl group or a t-butyldimethylsilyl group.

Regarding Ys in each formula in the repeating unit (I), the introduction rate of a group other than a hydrogen atom (an alkyl group and/or an alkyl silyl group) is not particularly limited. However, when at least some of Ys are alkyl groups and/or alkyl silyl groups, 25% or more (more preferably 50% or more, and further preferably 75% or more) of the summed amount of Ys in the repeating unit (I) is preferably alkyl groups and/or alkyl silyl groups (note that in this case, Ys other than the alkyl groups and/or alkyl silyl groups are hydrogen atoms). When 25% or more of the summed amount of each Y in the repeating unit (I) is an alkyl group and/or an alkyl silyl group, the storage stability of the polyimide precursor resin tends to be better.

In addition, in the general formulae (4) to (6), one of a bonding arm represented by *1 and a bonding arm represented by *2 is bonded to a carbon atom a forming the norbornane ring (a carbon atom denoted by sign a) and the other of the bonding arm represented by *1 and the bonding arm represented by *2 is bonded to a carbon atom b forming the norbornane ring (a carbon atom denoted by sign b). In addition, in the general formulae (4) to (6), one of a bonding arm represented by *3 and a bonding arm represented by *4 is bonded to a carbon atom c forming the norbornane ring (a carbon atom denoted by sign c) and the other of the bonding arm represented by *3 and the bonding arm represented by *4 is bonded to a carbon atom d forming the norbornane ring (a carbon atom denoted by d). Note that the bonding arms represented by *1 to *4 each may take an exo three-dimensional configuration or an endo three-dimensional configuration relative to the norbornane ring to which the bonding arm is bonded.

Although the three-dimensional configurations of the bonding arms represented by *1 to *4 are not particularly limited, each bonding arm preferably takes an exo three-dimensional configuration from the viewpoint that it is possible to further improve the reactivity and further reduce the linear expansion coefficient.

Note that in a case where the polyimide precursor resin is obtained under mild reaction conditions, the bonding arms *1 to *4 in the repeating unit normally take an exo three-dimensional configuration relative to the norbornane methylene head. This is because in the case where the polyimide precursor resin is formed under mild reaction conditions, the three-dimensional configuration of the raw material compound (an acid dianhydride ring bonded to the norbornane ring basically takes an exo three-dimensional configuration relative to the norbornane methylene head) is maintained and the bonding arms *1 to *4 take an exo three-dimensional configuration relative to the norbornane methylene head. In this way, when an exo three-dimensional configuration is maintained, it is possible to allow the imidization reaction (condensation reaction) step to more efficiently proceed when a polyimide is formed by using a polyimide precursor resin having such a three-dimensional configuration. Moreover, the configurations of the bonding arm *1 and the bonding arm *3 may be a cis configuration or a trans configuration relative to the polymer main chain. Although depending on the configurations of the bonding arm *1 and the bonding arm *3, the configurations of the bonding arm *2 and the bonding arm *4 may also be a cis configuration or a trans configuration. It is known in general that when the configurations of the bonding arm *1 and the bonding arm *3 are the trans configuration relative to the polymer main chain, physical properties such as linear expansion coefficient are improved.

The polyimide precursor resin of the present invention is a polyimide precursor resin comprising a repeating unit (I) represented by the general formula (4). In the repeating unit (I) represented by the general formula (4), the portion of the norbornane rings and the cycloalkanone ring in the formula (4) can take 6 three-dimensional structures as described in the formulae (Si) to (S6), based on the three-dimensional configurations of the two norbornane rings and the cycloalkanone ring. Specifically, the repeating unit (I) can contain a repeating unit having a trans-exo-exo three-dimensional structure represented by the formula (Si) (corresponding to the repeating unit (I-A) having the three-dimensional structure represented by the general formula (5)); a repeating unit having a cis-exo-exo three-dimensional structure represented by the formula (S2) (corresponding to the repeating unit (I-B) having the three-dimensional structure represented by the general formula (6)); a repeating unit having a trans-endo-endo three-dimensional structure represented by the formula (S3); a repeating unit having a cis-endo-endo three-dimensional structure represented by the formula (S4); a repeating unit having a trans-exo-endo three-dimensional structure represented by the formula (S5); and a repeating unit having a cis-exo-endo three-dimensional structure represented by the formula (S6).

As described above, the repeating unit (I) represented by the general formula (4) can contain 6 repeating units having different three-dimensional structures. In the polyimide precursor resin of the present invention, the content of the repeating unit (I-A) having the three-dimensional structure represented by the general formula (5) in the repeating unit (I) is 40% by mol to 98% by mol (more preferably 45% by mol to 98V by mol, further preferably 65% by mol to 97% by mol, particularly preferably 75% by mol to 96% by mol, and most preferably 80% by mol to 96% by mol) relative to the total amount of the repeating unit (I) (the total amount of the 6 repeating units having different three-dimensional structures). If the content of the repeating unit (I-A) is less than the lower limit, it is difficult to obtain lower values of the linear expansion coefficient and the dielectric dissipation factor of the polyimide when the polyimide is prepared by using the precursor resin. On the other hand, if the content of the repeating unit (I-A) exceeds the upper limit, the polyimide prepared by using the precursor resin tends to be brittle.

In addition, in the polyimide precursor resin of the present invention, the content of the repeating unit (I-B) having the three-dimensional structure represented by the general formula (6) in the repeating unit (I) is 2% by mol to 60% by mol (more preferably 2 to 55% by mol, further preferably 3% by mol to 35% by mol, particularly preferably 4% by mol to 25% by mol, and most preferably 4% by mol to 20% by mol) relative to the total amount of the repeating unit (I) (the total amount of the 6 repeating units having different three-dimensional structures). If the content of the repeating unit (I-B) is less than the lower limit, it is difficult to obtain lower values of the linear expansion coefficient and the dielectric dissipation factor of the polyimide when the polyimide is prepared by using the precursor resin. On the other hand, if the content of the repeating unit (I-B) exceeds the upper limit, the polyimide prepared by using the precursor resin tends to be brittle.

Moreover, in the polyimide precursor resin of the present invention, the summed amount of the repeating units (I-A) and (I-B) in the repeating unit (I) is 42V by mol or more (more preferably 50% by mol or more, further preferably 74% by mol or more, particularly preferably 85% by mol or more, and most preferably 90V by mol or more) relative to the total amount of the repeating unit (I) (the total amount of the 6 repeating unit having different three-dimensional structure). If the summed amount of the repeating units (I-A) and (I-B) is less than the lower limit, it is difficult to obtain lower values of the linear expansion coefficient and the dielectric dissipation factor of the polyimide when the polyimide is prepared by using the precursor resin.

Further, in the polyimide precursor resin of the present invention, when the repeating unit (I) contains a repeating unit having a three-dimensional structure other than those of the repeating units (I-A) and (I-B), the summed amount of the repeating unit having the trans-endo-endo three-dimensional structure and the repeating unit having the cis-endo-endo three-dimensional structure is preferably 10% by mol or less, and more preferably 0 to 5% by mol, relative to the total amount of the repeating unit (I). Furthermore, when the repeating unit (I) contains a repeating unit other than the repeating units (I-A) and (I-B), the summed amount of the repeating unit having the trans-exo-endo three-dimensional structure and the repeating unit having the cis-exo-endo three-dimensional structure is preferably 30% by mol or less, and more preferably 0 to 20% by mol, relative to the total amount of the repeating unit (I). If the summed amount of the repeating units having three-dimensional structures other than those of the repeating units (I-A) and (I-B), which can be contained in the repeating unit (I), exceeds the upper limit, it is difficult to obtain lower values of the linear expansion coefficient and the dielectric dissipation factor of the polyimide when the polyimide is prepared by using the precursor resin.

In addition, in the polyimide precursor resin, the content of the repeating unit (I) represented by the general formula (4) is more preferably 50 to 100% by mol (more preferably 70 to 100% by mol, and further preferably 80 to 100% by mol). Note that the polyimide precursor resin may contain another repeating unit as long as it does not impair the effects of the present invention. Such other repeating unit includes, for example, a repeating unit derived from a tetracarboxylic dianhydride other than the tetracarboxylic dianhydride represented by the general formula (1), and the like. As such a tetracarboxylic dianhydride other than the tetracarboxylic dianhydride represented by the general formula (1), any known tetracarboxylic dianhydride may be used as appropriate, and for example, those described in paragraph [0230] of International Publication No. WO2015/163314 may be used as appropriate.

Such a polyimide precursor resin (polyamic acid) has an intrinsic viscosity [η] of preferably 0.05 to 3.0 dL/g, and more preferably 0.1 to 2.0 dL/g. If the intrinsic viscosity [η] is less than 0.05 dL/g, when a film-shaped polyimide is produced using this, the film thus obtained tends to be brittle. On the other hand, the intrinsic viscosity [η] exceeds 3.0 dL/g, the excessively high viscosity leads to low processability, so that for example, when a film is produced, it is difficult to obtain a uniform film. In addition, as the intrinsic viscosity [η], a value obtained by preparing a measurement sample (solution) in which the polyamic acid is dissolved in N,N-dimethylacetamide at a concentration of 0.5 g/dL, and measuring the viscosity of the measurement sample using a kinematic viscometer under a temperature condition of 30° C. is employed. Note that as the kinematic viscometer, an automatic viscometer (manufactured by RIGO CO., LTD under the trade name of “VMC-252”) may be used.

In addition, a preferable method for producing the polyimide precursor resin of the present invention includes a method for producing a polyimide precursor resin by reacting the tetracarboxylic dianhydride of the present invention and an aromatic diamine represented by a formula: H₂N—R¹⁰—NH₂ [R¹⁰ in the formula have the same definition as that of R¹⁰ in the general formulae (5) and (6)]. As the aromatic diamine, a known one (for example, an aromatic diamine described in paragraph [0039] of Japanese Unexamined Patent Application Publication No. 2018-44180, or the like) may be used as appropriate, and the aromatic diamines described during the explanation of R¹⁰ may be used as appropriate. In addition, the conditions for reacting the tetracarboxylic dianhydride of the present invention and an aromatic diamine are not particularly limited, and known conditions as used in preparing a polyamic acid may be employed as appropriate (for example, known conditions as employed in a method described in paragraphs [0134] to [0156] of WO 2011/099518, a method described in paragraphs [0215] to [0235] of WO 2015/163314, and the like (conditions such as a solvent, a reaction temperature, and the like) may be employed as appropriate). Note that when the tetracarboxylic dianhydride of the present invention and the aromatic diamine are reacted, the repeating unit (I) may be a repeating unit of a polyamic acid in which both of Ys are hydrogen atoms. As a producing method in the case of producing a polyimide precursor resin containing the repeating unit (I) in which Ys are other than hydrogen atoms, for example, a method for producing in the same manner as a method described in paragraphs [0165] to [0174] of International Publication No. WO2018/066522 may be employed as appropriate except for using the tetracarboxylic dianhydride of the present invention as a tetracarboxylic dianhydride.

In addition, when a polyimide precursor resin is formed by reacting the tetracarboxylic dianhydride of the present invention and the aromatic diamine, it basically is possible to allow the repeating units (I-A) and (I-B) to be contained in the same ratios as the content ratios of the isomer (A) and the isomer (B) contained in the tetracarboxylic dianhydride of the present invention (because during the reaction, the three-dimensional structures are maintained). This point will be briefly described. First, the repeating unit (I) represented by the general formula (4) can be formed, derived from the compound represented by the general formula (1) and an aromatic diamine. In addition, since during the reaction between the compound represented by the general formula (1) and an aromatic diamine, the three-dimensional structure of the norbornane rings and the cycloalkanone ring in each stereoisomer in the compound represented by the general formula (1) (the three-dimensional structures represented by the formulae (Si) to (S6): the cis or trans configuration of the norbornane ring, and the three-dimensional configuration (exo or endo three-dimensional configuration) of the carbonyl group (C═O) in the cycloalkanone ring relative to the norbornane rings are basically maintained as they are, it is possible to form the repeating unit (I-A) derived from the reaction between the isomer (A) in the compound represented by the general formula (1) with the aromatic diamine, and to form the repeating unit (I-B) derived from the reaction between the isomer (B) in the compound represented by the general formula (1) with the aromatic diamine. Hence, by reacting the tetracarboxylic dianhydride of the present invention and the aromatic diamine, it is possible to easily prepare the polyimide precursor resin of the present invention as the reaction product. Note that to change the content of the repeating unit (I), another type of tetracarboxylic dianhydride may be used together with the tetracarboxylic dianhydride of the present invention.

When a polyimide precursor resin is formed by reacting the tetracarboxylic dianhydride of the present invention and the aromatic diamine, a monofunctional carboxylic acid anhydride or a monofunctional aromatic amine may be added as a molecular weight controller. The monofunctional carboxylic acid anhydride includes, for example, succinic anhydride, succinic anhydrides, maleic anhydride, maleic anhydrides, citraconic anhydride, 1,2-cyclohexanedicarboxylic anhydride, cis-1,2-cyclohexanedicarboxylic anhydride, trans-1,2-cyclohexanedicarboxylic anhydride, 3-methylcyclohexane-1,2-dicarboxylic anhydride, 4-methylcyclohexane-1,2-dicarboxylic anhydride, bicyclo[2.2.2]oct-5-ene-2,3-dicarboxylic anhydride, bicyclo[2.2.1]hept-5-ene-2,3-dicarboxylic anhydride, 1-cyclohexene-1,2-dicarboxylic anhydride, phthalic anhydride, 1,2-naphthalenedicarboxylic anhydride, 2,3-naphthalenedicarboxylic anhydride, 3-methylphthalic anhydride, 4-methylphthalic anhydride, 4-tert-butyl phthalic anhydride, 3-fluorophthalic anhydride, 4-fluorophthalic anhydride, 3-chlorophthalic anhydride, 4-chlorophthalic anhydride, 3-bromophthalic anhydride, 4-bromophthalic anhydride, and the like. In addition, the monofunctional aromatic amine includes, for example, aniline, methylaniline, dimethylaniline, trimethylaniline, ethylaniline, tert-butylaniline, fluoroaniline, chloroaniline, bromoaniline, aminonaphthalene, and the like. One of the monofunctional carboxylic acid anhydride and the monofunctional aromatic amine may be used alone or both of them may be used in combination. In addition, one of the monofunctional carboxylic acid anhydrides and the monofunctional aromatic amines may be used alone or two or more of them may be used in combination. When both of the monofunctional carboxylic acid anhydride and the monofunctional aromatic amine are used, multiple types of each may be combined as a mixture to be used. In addition, the amount of the monofunctional carboxylic acid anhydride to be added and the amount of the monofunctional aromatic amine to be added are, although depending on target molecular weights, preferably 0.0001 to 10% by mol, and further preferably 0.01 to 1% by mol, relative to the tetracarboxylic dianhydride of the present invention and the aromatic diamine.

Note that the polyimide precursor resin (preferably, a polyamic acid) of the present invention may be added to an organic solvent to be used as a polyimide precursor resin solution (varnish). Hereinafter, a polyimide precursor resin solution of the present invention preferable as the polyimide precursor resin solution (varnish) will be described.

### [Polyimide Precursor Resin Solution of Present Invention]

The polyimide precursor resin solution of the present invention comprises the polyimide precursor resin of the present invention and an organic solvent.

The organic solvent to be used for the polyimide precursor resin solution (varnish) is not particularly limited, and a known one may be used as appropriate, and for example, solvents described in paragraph [0175] and paragraphs [0133] to [0134] of WO 2018/066522, and the like may be used as appropriate. The organic solvent includes, for example, polar aprotic solvents such as N-methyl-2-pyrrolidone, N,N-dimethylacetamide, N,N-dimethylformamide, dimethyl sulfoxide, γ-butyrolactone, propylene carbonate, tetramethylurea, 1,3-dimethyl-2-imidazolidinone, hexamethylphosphoric triamide, and pyridine; phenol solvents such as m-cresol, xylenol, phenol, and halogenated phenol; ether solvents such as tetrahydrofuran, dioxane, cellosolve, and glyme; aromatic solvents such as benzene, toluene, and xylene; ketone solvents such as cyclopentanone and cyclohexanone; nitrile solvents such as acetonitrile and benzonitrile; and the like. One of such organic solvents may be used alone or two or more of them may be mixed and used.

The content of the polyimide precursor resin in the polyimide precursor resin solution is not particularly limited, but is preferably 1 to 80% by mass, and more preferably 5 to 50% by mass. If the content is less than the lower limit, it tends to be difficult to use the polyimide precursor resin solution as a varnish for producing a polyimide film. On the other hand, if the content exceeds the upper limit, it tends to be difficult to use the polyimide precursor resin solution as a varnish for producing a polyimide film. Note that the polyimide precursor resin solution can be favorably used as a resin solution (varnish) for producing the polyimide of the present invention, and can be favorably used for producing the polyimide in various forms. For example, it is possible to easily produce a film-shaped polyimide by applying the polyimide precursor resin solution onto various substrates and solidifying the polyimide precursor resin solution through imidization.

Note that the method for preparing the polyimide precursor resin solution of the present invention is not particularly limited, and for example, the polyimide precursor resin solution may be prepared by in the method for producing a polyimide precursor resin of the present invention, reacting the tetracarboxylic dianhydride of the present invention and the aromatic diamine in the organic solvent, and using a reaction liquid obtained after the reaction as the polyimide precursor resin solution as it is. A known additive such as an antioxidant, an ultraviolet absorber, or a filler may be added to the polyimide precursor resin solution as appropriate in accordance with the purpose of use and the like.

### [Polyimide]

The polyimide of the present invention is a polyimide comprising a repeating unit (II) represented by the general formula (7), wherein

a content of a repeating unit (II-A) having a three-dimensional structure represented by the general formula (8) is 40% by mol to 98% by mol relative to a total amount of the repeating unit (II),

a content of a repeating unit (II-B) having a three-dimensional structure represented by the general formula (9) is 2% by mol to 60% by mol relative to the total amount of the repeating unit (II), and

a summed amount of the repeating units (II-A) and (II-B) is 42% by mol or more relative to the total amount of the repeating unit (II).

R¹, R², R³, and n in the general formulae (7) to (9) have the same definitions as those of R¹, R², R³, and n in the general formulae (1) to (3), respectively (the preferred ones and preferred conditions also have the same definitions). In addition, R¹⁰ in the general formulae (7) to (9) has the same definition as that of R¹⁰ in the general formulae (4) to (6)) (the preferred ones, preferred conditions, and the like also have the same definitions).

The polyimide of the present invention is a polyimide comprising a repeating unit (II) represented by the general formula (7). In the repeating unit (II) represented by the general formula (7), the portion of the norbornane rings and the cycloalkanone ring in the formula (7) can take 6 three-dimensional structures as described in the formulae (Si) to (S6), based on the three-dimensional configurations of the two norbornane rings and the cycloalkanone ring. Specifically, the repeating unit (II) can contain a repeating unit having a trans-exo-exo three-dimensional structure represented by the formula (Si) (corresponding to the repeating unit (II-A) having the three-dimensional structure represented by the general formula (8)); a repeating unit having a cis-exo-exo three-dimensional structure represented by the formula (S2) (corresponding to the repeating unit (II-B) having the three-dimensional structure represented by the general formula (9)); a repeating unit having a trans-endo-endo three-dimensional structure represented by the formula (S3); a repeating unit having a cis-endo-endo three-dimensional structure represented by the formula (S4); a repeating unit having a trans-exo-endo three-dimensional structure represented by the formula (S5); and a repeating unit having a cis-exo-endo three-dimensional structure represented by the formula (S6).

As described above, the repeating unit (II) represented by the general formula (7) can contain 6 repeating unit having different three-dimensional structures. In the polyimide of the present invention, the content of the repeating unit (II-A) having the three-dimensional structure represented by the general formula (8) in the repeating unit (II) is 40% by mol to 98% by mol (more preferably 45% bymol to 98% bymol, further preferably 65% by mol to 97% by mol, particularly preferably 75% by mol to 96% by mol, and most preferably 80% by mol to 96% by mol) relative to the total amount of the repeating unit (II) (the total amount of the 6 repeating units having different three-dimensional structures). If the content of the repeating unit (II-A) is less than the lower limit, it is difficult to obtain lower values of the linear expansion coefficient and the dielectric dissipation factor. On the other hand, if the content of the repeating unit (II-A) exceeds the upper limit, the resin itself tends to be brittle.

In addition, in the polyimide of the present invention, the content of the repeating unit (II-B) having the three-dimensional structure represented by the general formula (9) in the repeating unit (II) is 2% by mol to 60% by mol (more preferably 2 to 55% by mol, further preferably 3% by mol to 35% by mol, particularly preferably 4% by mol to 25% by mol, and most preferably 4% by mol to 20% by mol) relative to the total amount of the repeating unit (II) (the total amount of the 6 repeating units having different three-dimensional structures). If the content of the repeating unit (II-B), it is difficult to obtain lower values of the linear expansion coefficient and the dielectric dissipation factor. On the other hand, if the content of the repeating unit (II-B) exceeds the upper limit, the resin itself tends to be brittle.

Moreover, in the polyimide of the present invention, the summed amount of the repeating units (II-A) and (II-B) in the repeating unit (II) is 42% by mol or more (more preferably 50% by mol or more, further preferably 74% by mol or more, particularly preferably 85% by mol or more, and most preferably 90% by mol or more) relative to the total amount of the repeating unit (II) (the total amount of the 6 repeating units having different three-dimensional structures) If the summed amount of the repeating units (II-A) and (II-B) is less than the lower limit, it is difficult to obtain lower values of the linear expansion coefficient and the dielectric dissipation factor.

Further, in the polyimide of the present invention, when the repeating unit (II) contains a repeating unit having a three-dimensional structure other than those of the repeating units (II-A) and (II-B), the summed amount of the repeating unit having the trans-endo-endo three-dimensional structure and the repeating unit having the cis-endo-endo three-dimensional structure is preferably 10% by mol or less, and more preferably 0 to 5% by mol, relative to the total amount of the repeating unit (II). Furthermore, when the repeating unit (II) contains a repeating unit other than the repeating units (II-A) and (II-B), the summed amount of the repeating unit having the trans-exo-endo three-dimensional structure and the repeating unit having the cis-exo-endo three-dimensional structure is preferably 30% by mol or less, and more preferably 0 to 20% by mol, relative to the total amount of the repeating unit (II). If the summed amount of the repeating unit having three-dimensional structure other than those of the repeating units (II-A) and (II-B), which can be contained in the repeating unit (II), exceeds the upper limit, it is difficult to obtain lower values of the linear expansion coefficient and the dielectric dissipation factor.

In addition, in such a polyimide, the content of the repeating unit (II) represented by the general formula (7) is more preferably 50 to 100% by mol (more preferably 70 to 100% by mol, further preferably 80 to 100% by mol). Note that the polyimide may contain another repeating unit as long as it does not impair the effects of the present invention. Such other repeating unit includes, for example, a repeating unit derived from a tetracarboxylic dianhydride other than the tetracarboxylic dianhydride represented by the general formula (1), and the like. As such a tetracarboxylic dianhydride other than the tetracarboxylic dianhydride represented by the general formula (1), any known tetracarboxylic dianhydride may be used as appropriate, and for example, those described in paragraph [0230] of WO 2015/163314 may be used as appropriate.

Note that although the three-dimensional configuration of the imide ring bonded to each of the two norbornane rings in the repeating unit (II) represented by the general formula (7) is not particularly limited and each imide ring may take an exo three-dimensional configuration or an endo three-dimensional configuration relative to the norbornane ring to which the imide ring is bonded, each imide ring preferably takes an exo three-dimensional configuration from the viewpoint that it is possible to further improve the reactivity and further reduce the linear expansion coefficient.

In addition, such a polyimide has a glass transition temperature (Tg) of preferably 250° C. or more, more preferably 290 to 500° C., and particularly preferably 330 to 500° C. If the glass transition temperature (Tg) is less than the lower limit, it tends to be difficult to obtain a sufficiently high heat resistance. On the other hand, if the glass transition temperature (Tg) exceeds the upper limit, it tends to be difficult to produce a polyimide having such properties. Note that the glass transition temperature (Tg) can be measured by using a thermomechanical analyzer (manufactured by Rigaku corporation under the trade name of “TMA8311”).

In addition, such a polyimide has a 5% weight loss temperature of preferably 350° C. or more, and more preferably 450 to 600° C. Note that the 5% weight loss temperature can be obtained by gradually heating a polyimide from room temperature (25° C.) while causing a nitrogen gas to flow under a nitrogen gas atmosphere and measuring a temperature at which the weight of the sample used is reduced by 5%. Moreover, such a polyimide has a softening temperature of preferably 270° C. or more, and more preferably 320 to 500° C. Note that the softening temperature can be measured by using a thermomechanical analyzer (manufactured by Rigaku corporation under the trade name of “TMA8311”) in a penetration mode. In addition, such a polyimide has a thermal decomposition temperature (Td) of preferably 400° C. or more, and more preferably 450 to 600° C. Note that the thermal decomposition temperature (Td) can be obtained by using a TG/DTA220 thermogravimetric analyzer (manufactured by SII NanoTechnology Inc.) to measure the temperature at the intersection of a tangent drawn on the decomposition curve before and after the thermal decomposition under a nitrogen atmosphere and under the condition of a rate of temperature rise of 10° C./min.

Moreover, the number-average molecular weight (Mn) of the polyimide is preferably 1000 to 1000000 in terms of polystyrene. In addition, the weight-average molecular weight (Mw) of the polyimide is preferably 1000 to 5000000 in terms of polystyrene. Furthermore, the molecular weight distribution (Mw/Mn) of the polyimide is preferably 1.1 to 5.0. Note that the molecular weight (Mw or Mn) and the molecular weight distribution (Mw/Mn) of the polyimide can be obtained by using a gel permeation chromatography as a measurement device and converting measured data in terms of polystyrene.

In addition, when a film is formed from the polyimide, the film preferably has a sufficiently high transparency, and more preferably has a total luminous transmittance of 80% or more (further preferably 85% or more, and particularly preferably 87% or more). The total luminous transmittance can be obtained by conducting a measurement according to JIS K7361-1 (published in 1997).

In addition, the polyimide has a linear expansion coefficient of preferably 0 to 70 ppm/K, and more preferably 5 to 40 ppm/K. If the linear expansion coefficient exceeds the upper limit, when a polyimide film or molded article is produced from the polyimide precursor resin, the film obtained tends to shrink or curl, and further when the film is combined with a metal or inorganic material having a linear expansion coefficient within a range of 5 to 20 ppm/K as a composite material, separation tends to easily occur due to a heat history. On the other hand, if the linear expansion coefficient is less than the lower limit, the polyimide is so rigid that the breaking elongation is low and the flexibility thus decreases. As the linear expansion coefficient of the polyimide, a value obtained by forming a polyimide film having a size of 20 mm in length and 5 mm in width (although the thickness of the film is not particularly limited because the thickness does not affect the measured value, the thickness is preferably 5 to 80 μm) as a measurement sample, and using a thermomechanical analyzer (for example, the trade name “TMA8311” manufactured by Rigaku corporation) as a measurement device to measure a change in vertical length of the sample at 50° C. to 200° C. while employing the condition of a rate of temperature rise of 5° c./min. in tension mode (49 mN) under a nitrogen atmosphere, and obtaining an average value of the change in length per 1° C. from the change in length in a temperature range of 100° C. to 200° C. is employed.

In addition, the polyimide more preferably has a haze (turbidity) of 5 to 0 (further preferably 4 to 0, particularly preferably 3 to 0). Moreover, the polyimide more preferably has a yellowness index (YI) of 7 to 0 (further preferably 6 to 0, particularly preferably 4 to 0, and most preferably 3 to 0). The haze (turbidity) can be obtained by conducting a measurement according to JIS K7136 (published in 2000), and the yellowness index (YI) can be obtained by conducting a measurement according to ASTM E313-05 (published in 2005).

In addition, the polyimide preferably has a dielectric dissipation factor (tan δ) of 0.022 or less (more preferably 0.02 to 0.005) at a frequency of 10 GHz. If the dielectric dissipation factor (tan δ) exceeds the upper limit, the degree of electric energy loss in a dielectric tends to increase. In addition, the polyimide preferably has a relative permittivity (εr) of 3 or less (more preferably 2.9 to 2.0) at a frequency of 10 GHz. If the relative permittivity (εr) exceeds the upper limit, when the polyimide is used as a high-frequency substrate material, the signal delay time tends to increase. Note that the dielectric dissipation factor (tan δ) and the relative permittivity (εr) can be measured according to ASTM D2520.

In addition, the method for producing the polyimide of the present invention is not particularly limited, and includes, for example, a method for producing a polyimide by reacting the tetracarboxylic dianhydride of the present invention and an aromatic diamine represented by a formula: H₂N—R¹⁰—NH₂ [in the formula, R¹⁰ has the same definition as that of R¹⁰ in the general formula (4) to (7)]. Note that as the aromatic diamine, a known one (for example, the aromatic diamine described in paragraph [0039] of JP 2018-44180, or the like) may be used as appropriate, and the aromatic diamine described for explaining R¹⁰ may be favorably used.

As conditions for reacting the tetracarboxylic dianhydride of the present invention and the aromatic diamine, the conditions employed in the known methods for producing a polyimide by reacting a tetracarboxylic dianhydride and a diamine (for example, the method described in WO 2011/099518, the method described in WO 2015/163314, the method described in JP 2018-044180, the method described in WO 2018/066522, and the like) may be employed as appropriate. As described above, the polyimide of the present invention can be produced in the same manner as that in the known methods for producing a polyimide by reacting a tetracarboxylic dianhydride and a diamine except that the tetracarboxylic dianhydride of the present invention is used as the tetracarboxylic dianhydride as a monomer. Note that when the method for producing a polyimide by reacting the tetracarboxylic dianhydride of the present invention and the aromatic diamine is employed, a polyimide may be produced by reacting the tetracarboxylic dianhydride of the present invention and the aromatic diamine to prepare the polyamic acid of the present invention, and thereafter imidizing the polyamic acid. The imidization method is not particularly limited, and conditions and the like employed in known methods that capable of imidizing a polyamic acid (for example, the method described in paragraphs [0134] to [0156] of WO 2011/099518, and the like) may be employed as appropriate. Note that in the imidization of a polyamic acid, from the viewpoint of improving the molecular weight and improving the imidization rate by solid phase polymerization, it is preferable to use a phosphorus-based compound such as triphenyl phosphite, triphenyl phosphate, triphenylphosphine, and triphenylphosphine oxide in an amount of approximately 0.1 to 50 parts by mass relative to 100 parts by mass of the polyamic acid. In addition, from the same viewpoint, in the imidization of a polyamic acid, imidazole-based compounds such as imidazole, methylimidazole, 1-methylimidazole, 2-methylimidazole, dimethylimidazole, 1,2-dimethylimidazole, trimethylimidazole, tetramethylimidazole, tert-butylimidazole, phenylimidazole, 2-phenylimidazole, benzylimidazole, benzimidazole, ethylimidazole, propylimidazole, butylimidazole, fluoroimidazole, chloroimidazole, and bromoimidazole; histidine-based compounds such as 2-amino-3-(1H-imidazol-4-yl)propanoic acid, β-phenyl-1H-imidazole-1-propionic acid, β-(4-methoxyphenyl)-1H-imidazole-1-propionic acid, β-(4-methylphenyl)-1H-imidazole-1-propionic acid, β-alanyl-L-histidine, L-histidine, and N-t-butoxycarbonyl-L-histidine; imidazole peptide-based compounds such as carnosine, anserine, balenine, and homocarnosine; and tertiary amine-based compounds such as trimethylamine, triethylamine, N,N-diisopropylethylamine, tetramethylethylenediamine, and pyridine; may be favorably used one of the compounds such as imidazole-based compounds and histidine-based compounds may be used alone or two or more of them may be used in combination. In addition, these compounds are preferably used such that the summed amount thereof is in a ratio of approximately 0.1 to 50 parts by mass relative to 100 parts by mass of the polyamic acid.

Note that in the preparation of the polyimide, it is possible to almost simultaneously conduct a polyamic acid preparation step and the subsequent imidization step by using a compound such as the imidazole-based compounds, the tertiary amine-based compounds, the histidine-based compounds, and the imidazole peptide-based compounds as a so-called reaction accelerator, and for example, heating a mixture liquid containing the tetracarboxylic dianhydride of the present invention, the aromatic diamine, the organic solvent, and the reaction accelerator (the imidazole-based compound or the like) to 100 to 250° C. to thereby prepare a polyimide in the organic solvent.

In addition, when a polyimide is formed by reacting the tetracarboxylic dianhydride of the present invention and the aromatic diamine, it is possible to allow the repeating units (II-A) and (II-B) to be contained in the same ratios as the content ratios of the isomer (A) and the isomer (B) contained in the tetracarboxylic dianhydride of the present invention (because during the reaction, the three-dimensional structures are maintained). This point will be briefly described. First, the repeating unit (II) represented by the general formula (7) can be formed, derived from the compound represented by the general formula (1) and an aromatic diamine. In addition, since during the reaction between the compound represented by the general formula (1) and an aromatic diamine, the three-dimensional structure of the norbornane rings and the cycloalkanone ring in each stereoisomer in the compound represented by the general formula (1) (the three-dimensional structures represented by the formulae (S1) to (S6)) are basically maintained as they are, it is possible to form the repeating unit (II-A) derived from the reaction between the isomer (A) in the compound represented by the general formula (1) and the aromatic diamine, and to form the repeating unit (II-B) derived from the reaction between the isomer (B) in the compound represented by the general formula (1) and the aromatic diamine. Hence, by reacting the tetracarboxylic dianhydride of the present invention and the aromatic diamine, it is possible to easily prepare the polyimide of the present invention as the reaction product. Note that to change the content of the repeating unit (II), another type of tetracarboxylic dianhydride may be used together with the tetracarboxylic dianhydride of the present invention.

In addition, since the polyimide of the present invention can have lower linear expansion coefficient while having sufficiently high transparency and heat resistance, the polyimide of the present invention can be used as a material and the like for producing, for example, films for flexible wiring boards, heat-resistant insulating tapes, electric wire enamels, protective coating agents for semiconductors, liquid crystal alignment films, transparent conductive films for organic ELs, flexible board films, flexible transparent conductive films, transparent conductive films for organic thin solar cells, transparent conductive films for dye-sensitized solar cells, flexible gas barrier films, films for touch panels, seamless polyimide belts for copiers (so-called transfer belts), transparent electrode substrates (transparent electrode substrates for organic ELs, transparent electrode substrates for solar cells, transparent electrode substrates for electronic papers, and the like), interlayer insulating films, sensor boards, image sensor boards, reflectors for light-emitting diodes (LEDs) (Reflectors for LED illumination: LED reflectors), covers for LED illumination, covers for LED reflector illumination, coverlay films, high-ductility composite boards, resists for semiconductors, lithium-ion batteries, organic memory substrates, organic transistor substrates, organic semiconductor substrates, color filter base materials, binders for all-solid-state batteries, and the like. In addition, since the polyimide of the present invention can have lower dielectric dissipation factor, the polyimide of the present invention can be favorably applied, for example, to high-frequency materials and the like. Moreover, besides the above-described applications, it is possible to use the polyimide of the present invention as appropriated for, for example, lenses for in-vehicle sensors, face-recognition lenses for mobile phones, high-temperature sterilized trays, optical communication connectors, automobile components, aerospace components, bearing components, sealing materials, bearing components, gear wheels, valve components, and the like by molding the polyimide of the present invention into a powder form, various types of compacts, and the like using the sufficiently high heat resistance.

### [Polyimide Solution]

The polyimide solution of the present invention comprises the polyimide of the present invention and an organic solvent.

As the organic solvent used for the polyimide solution, the same organic solvents as those for use in the above-described polyimide precursor resin solution can be favorably used. Note that, as the organic solvent used for the polyimide solution of the present invention, it is possible to use, for example, a halogen-based solvent having a boiling point of 200° C. or less (for example, dichloromethane (boiling point 40° C.), trichloromethane (boiling point 62° C.), carbon tetrachloride (boiling point 77° C.), dichloroethane (boiling point 84° C.), trichloroethylene (boiling point 87° C.), tetrachloroethylene (boiling point 121° C.), tetrachloroethane (boiling point 147° C.), chlorobenzene (boiling point 131° C.), o-dichlorobenzene (boiling point 180° C.), and the like) from the viewpoint of evaporation and removability of the solvent when the polyimide solution is used as a coating liquid.

In addition, from the viewpoints of solubility, film formation, productivity, industrial availability, presence or absence of existing equipment, and price, the organic solvent used for the polyimide solution is preferably N-methyl-2-pyrrolidone, N,N-dimethylformamide, N,N-dimethylacetamide, γ-butyrolactone, propylene carbonate, tetramethylurea, 1,3-dimethyl-2-imidazolidinone, or cyclopentanone, more preferably N-methyl-2-pyrrolidone, N,N-dimethylacetamide, y-butyrolactone, or tetramethylurea, and particularly preferably N,N-dimethylacetamide or y-butyrolactone. Note that one of those organic solvents may be used alone or two or more thereof may be used in combination.

In addition, the polyimide solution can also be preferably used as a coating liquid and the like for producing various processed products. For example, in the case of forming a film, a polyimide film may be formed by using the polyimide solution of the present invention as a coating liquid and applying the polyimide solution on a base material to obtain a coating film, and then removing the solvent. The coating method is not particularly limited, and it is possible to use known methods (such as the spin coating method, the bar coating method, and the dip coating method).

In the polyimide solution, the content (dissolution amount) of the polyimide is not particularly limited, but is preferably 1 to 75V by mass and more preferably 10 to 50% by mass. If the content is less than the lower limit, the film thickness after film formation tends to be thin in the case of use for film formation or the like. On the other hand, if the content exceeds the upper limit, a portion thereof tends to be insoluble in the solvent. Moreover, depending on the use purpose and the like, the polyimide solution may be further added with an additive such as an antioxidant (phenol-based antioxidants, phosphite-based antioxidants, thioether-based antioxidants, or the like), an ultraviolet absorber, a hindered amine light stabilizer, a nucleating agent, a resin additive (filler, talc, glass fiber, or the like), a flame retardant, a processability improver, or a lubricant. Note that these additives are not particularly limited, and known additives can be used as appropriate, and commercially-available additives may be used.

In addition, the polyimide solution may be prepared, for example, by reacting the tetracarboxylic dianhydride of the present invention and the aromatic diamine in the organic solvent to form a polyimide and using the reaction liquid as it is as the polyimide solution, and the preparation method is not particularly limited. Note that for the formation of a polyimide in an organic solvent, for example, a method for forming a polyimide in a solvent by heating a mixture liquid of at least one compound selected from the group consisting of the phosphorus-based compound, the imidazole-based compound, the tertiary amine-based compound, the histidine-based compound, and the imidazole peptide-based compound; the organic solvent; the tetracarboxylic dianhydride of the present invention; and the aromatic diamine to 100 to 250° C. may be employed.

### [Polyimide Film]

The polyimide film of the present invention is a cured product of at least one resin solution (varnish) selected from the group consisting of the polyimide precursor resin solution of the present invention and the polyimide solution of the present invention. As described above, the polyimide film of the present invention is obtained from the polyimide precursor resin solution of the present invention and/or the polyimide solution of the present invention.

The method for preparing the polyimide film of the present invention is not particularly limited, but for example, a method for obtaining a polyimide film made of a cured product by applying the polyimide precursor resin solution of the present invention (polyimide precursor resin varnish) onto a base material and removing a solvent, and then forming the cured product made of the polyimide through imidization, a method for obtaining a polyimide film which is a cured product of a polyimide by applying the polyimide solution of the present invention (polyimide varnish) onto a base material and removing the solvent, and the like may be employed as appropriate. Note that in the case where these methods are used, when the polyimide precursor resin solution of the present invention is applied onto a base material and a solvent is removed and thereafter imidization is conducted or when the polyimide solution of the present invention is applied onto a base material and a solvent is removed, a so-called calcining step (step of heating at a temperature of approximately 100 to 500° C. for 0.1 to 10 hours) may be applied. Such a calcining step makes it possible to obtain a polyimide film as a heat cured product.

The thickness of the polyimide film is not particularly limited, but is preferably 1 to 500 μm, and more preferably 5 to 200 μm. If the thickness is less than the lower limit, the strength tends to decrease, making it difficult to handle the polyimide film. On the other hand, if the thickness exceeds the upper limit, there is a tendency that multiple coatings are necessary in some cases, or the processing is complicated in some cases.

The form of the polyimide film only has to be a film form, is not particularly limited, and may be designed into various shapes (disk shape, cylindrical shape (obtained by processing the film into a cylindrical form), and the like). In the case where the polyimide film is produced by using the polyimide solution, it is also possible to more easily change the design. In addition, since the polyimide film of the present invention consequently is a film made of the polyimide of the present invention, the polyimide film of the present invention can be used as appropriate for the above-described various applications (such as, for example, films for flexible wiring boards, transparent conductive films for organic ELs, and flexible board films).

### EXAMPLES

Hereinafter, the present invention is more specifically described based on Examples and Comparative Examples, but the present invention is not limited to the following Examples.

### [Preparation of Tetracarboxylic Dianhydride]

### Synthesis Example 1

First, a 100 mL dropping funnel and a 2 L three-necked flask were prepared. After the atmosphere in the flask was set to a nitrogen atmosphere, cyclopentadiene (87.5 g, 1.3 mol), cyclopentanone (84.0 g, 1.0 mol), and formalin in a concentration of 37% by mass (163 g, the amount of formaldehyde: 2.0 mol) were added into the flask and were cooled down to 10° C. while being stirred to obtain a raw material mixture liquid. Next, a mixture liquid of diethylamine (14.5 g, 0.2 mol) and an aqueous solution of sulfuric acid in a concentration of 50% by mass (20.0 g, the amount of H₂SO₄: 0.1 mol) was added to the 100 mL dropping funnel. Subsequently, the dropping funnel was set to the two-necked flask, and the mixture liquid of diethylamine and sulfuric acid was dropped while the raw material mixture liquid in the flask was being cooled down to 10° C. to obtain a reaction liquid (note that Mannich bases were synthesized in the reaction liquid by the dropping step). After the reaction liquid was obtained in this way, the reaction liquid was transferred to a 1 L glass autoclave. Thereafter, the reaction liquid was heated to 150° C. while being vigorously stirred in the glass autoclave and reacted for 3 hours under a condition of a temperature of 150° C. After the reaction, when the reaction liquid was left to stand, the reaction liquid was separated into an organic layer and a water layer. Subsequently, the organic layer thus obtained was transferred to a separating funnel and diluted with 200 mL of toluene to obtain a toluene diluted solution. Next, the toluene diluted solution was washed with 200 mL of an aqueous solution of sulfuric acid in a concentration of 5% by mass, 200 mL of distilled water, 200 mL of an aqueous solution of sodium carbonate in a concentration of 10% by mass, and then, was further washed twice with 200 mL of distilled water. The toluene diluted solution after the washing was dried overnight with anhydrous sodium sulfate and the anhydrous sodium sulfate was filtrated and removed and the filtrate thus obtained was concentrated with an evaporator to obtain a crude product. The crude product thus obtained was of 61 g (yield 38%). Next, the crude product was added and dissolved to 244 g of methanol to obtain a solution. Then, the solution was cooled down to −20° C. precipitate a light yellow crystal (pale yellow crystal). The pale yellow crystal thus obtained was filtrated and was washed with methanol several times. Subsequently, the pale yellow crystal after the washing was dried with a vacuum dryer to obtain a product. The amount of the product thus obtained after the drying was 43 g. Note that as a result of NMR (¹H-NMR) measurement conducted on the product (crystal) thus obtained, the product obtained was confirmed to be a compound (5-norbornene-2-spiro-2′-cyclopentanone-5′-spiro-2″-5″-norbornene) represented by the following formula (X)

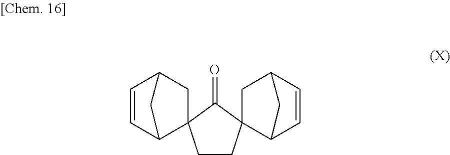 [SMILES: O=C1C2(CCC13CC1C=CC3C1)CC1C=CC2C1]

<Confirmation on Content Ratios of Stereoisomer in Compound Obtained in Synthesis Example 1>

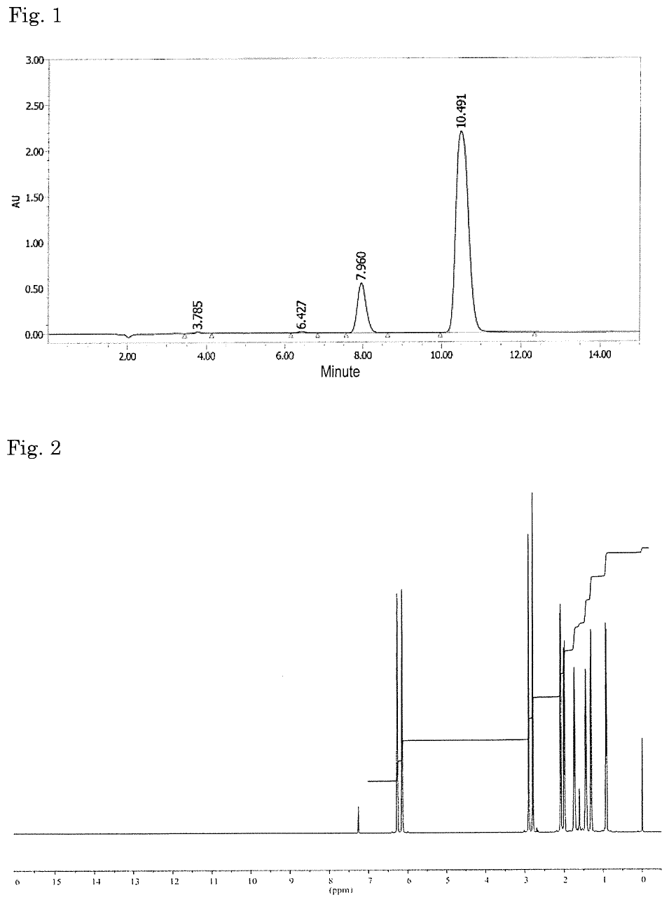

In order to confirm the ratios of isomers in the product (crystal) obtained in Synthesis Example 1, HPLC measurement as described below was conducted. Specifically, trade name “1200 Series” manufactured by Agilent Technologies was used as a measurement device, trade name “ZORBAX Eclipse XDB-C18 (particle size 5 μm, diameter 4.6 mm×length 150 mm)” manufactured by Agilent Technologies was used as a column, a mixture liquid of acetonitrile and water (acetonitrile/H₂O=70/30) was used as a solvent, the flow speed of the solvent was set to 1.0 mL/min., the detection wavelength of the diode array detector (DAD) was set to 210 nm, the temperature was set to 35° C., a sample obtained by adding 2 mg of the product (crystal) to 1.5 mL of the solvent was prepared, and then the HPLC measurement was conducted. FIG. 1 shows the result of the HPLC measurement.

In FIG. 1, 4 peaks were observed. Here, the area ratio of each peak was obtained. The peak at 10.49 minutes (hereinafter referred to as peak “(A)”) was 84.68% by area, the peak at 7.96 minutes (hereinafter referred to as “peak (B)”) was 14.65% by area, the peak at 6.43 minutes (hereinafter referred to as “peak (C)”) was 0.37% by area, and the peak at 3.79 minutes (hereinafter referred to as “peak (D)”) was 0.30% by area. Note that the peak (D) is the peak of a by-product (a compound in which one norbornene is bonded to a cyclopentanone).

Subsequently, the component of the peak (A) and the component of the peak (B) were recycled and separated using a preparative GPC (manufactured by Japan Analytical Industry Co., Ltd. under the trade name of “LC-918”, column: JAIGEL 1H, 2H (diameter 20×length 600 mm), solvent: chloroform, 3.5 ml/min., an UV detector (254 nm) and an RI detector were used in combination). Then, these components were separated through recycling 20 times. As a result of conducting the NMR (¹H-NMR) measurement on the component of the peak (A) and the component of the peak (B) thus obtained, it was confirmed that the component of the peak (A) was a mixture of two isomers (hereinafter, one of the isomers of the component of the peak (A) is referred to as the “peak (A-1) component” and the other isomer is referred to as the “peak (A-2) component”) and that the component of the peak (B) was also a mixture of two isomers (hereinafter, one of the isomers of the component of the peak (B) is referred to as the “peak (B-1) component” and the other isomer is referred to as the “peak (B-2) component”). In view of this, in order to conduct further separation and purification on the component of the peak (A) and the component of the peak (B), the peak (A-1) component, the peak (A-2) component, the peak (B-1) component, and the peak (B-2) component were recycled by using a preparative GPC (manufactured by Japan Analytical Industry Co., Ltd. under the trade name of “LaboACE LC-7080”, column: COSMOSIL Cholester Packed Column (diameter: 20×length 250 mm), solvent: acetonitrile/H₂O=80/20, the flow speed of the solvent: 10 ml/min., an UV detector (254 nm) and an RI detector were used in combination) through recycling 20 times and separated into each component (four isomers).

Measurement by a high-sensitivity NMR at 600 MHz and 800 MHz was conducted on the four isomers obtained by separation to determine the structure of each isomer. Specifically, the structure of each isomer was analyzed by using ¹H-NMR and ¹³C-NMR (including DEPT135) and two-dimensional NMR (COSY, HSQC, HMBC, NOESY). As a result of the structural analysis, it was confirmed that the component of the peak (A) was a mixture of a peak (A-1) component in which the portion of two norbornene rings and a cycloalkanone ring was composed of an isomer having a cis-exo-exo three-dimensional structure and a peak (A-2) component in which the portion of two norbornene rings and a cycloalkanone ring was composed of an isomer having a trans-exo-exo three-dimensional structure, and it was found that the ratio between the peak (A-1) component, which was the cis-exo-exo isomer, and the peak (A-2) component, which was the trans-exo-exo isomer, ([peak (A-1) component]:[peak (A-2) component]) was 1:2 in molar ratio. In addition, it was confirmed that the component of the peak (B) was a mixture of a peak (B-1) component in which the portion of two norbornene rings and a cycloalkanone ring was an isomer having a cis-exo-endo three-dimensional structure and a peak (B-2) component in which the portion of two norbornene rings and a cycloalkanone ring was an isomer having a trans-exo-endo three-dimensional structure, and it was found that the ratio between the peak (B-1) component, which was the cis-exo-endo isomer, and the peak (B-2) component, which was the trans-exo-endo isomer, ([peak (B-1) component]: [peak (B-2) component]) was 1:3.4 in molar ratio.

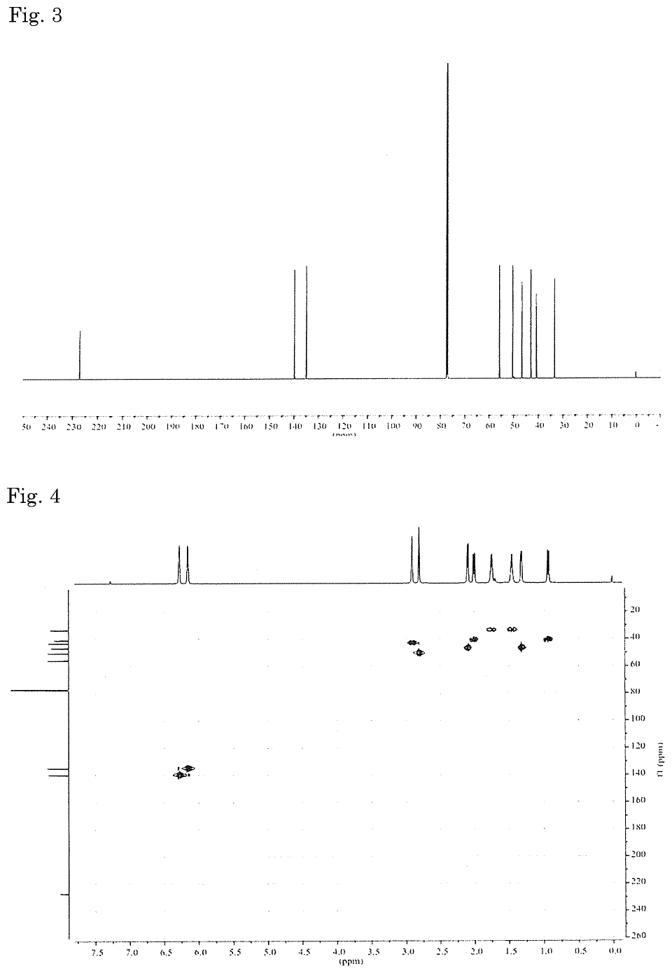

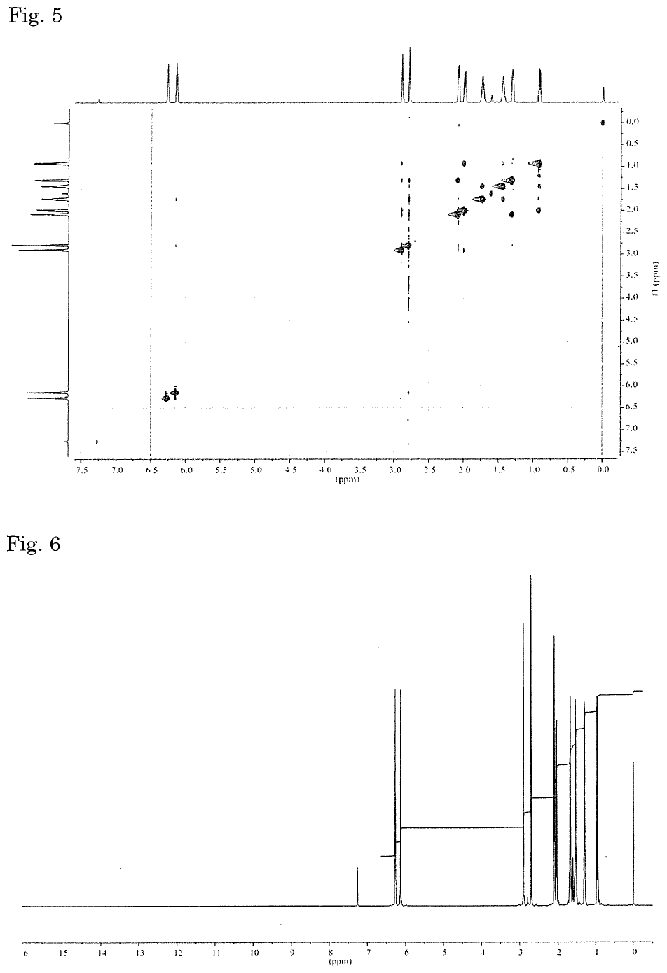

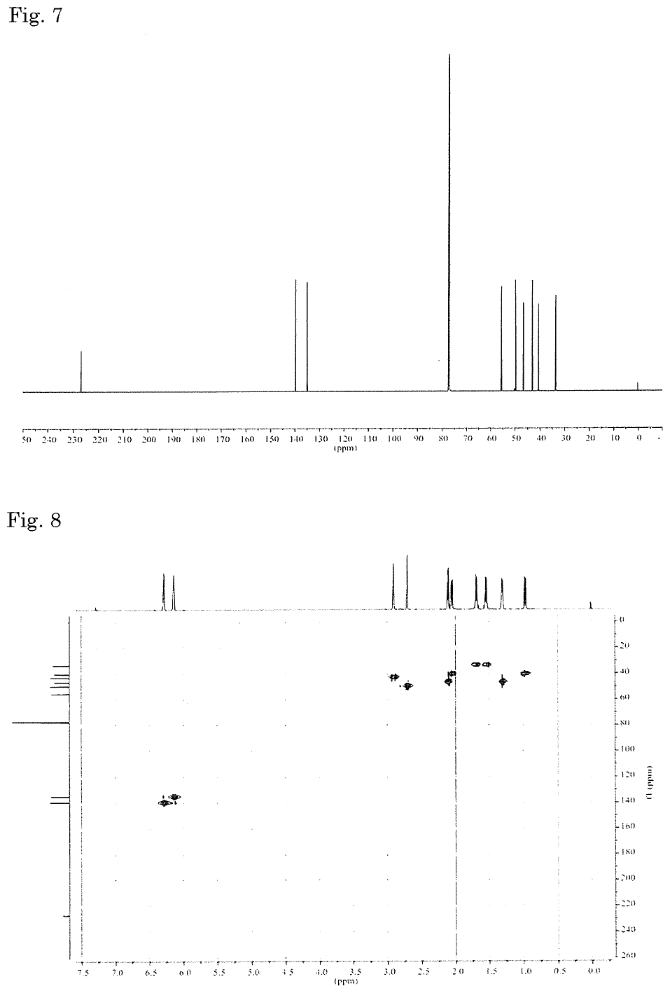

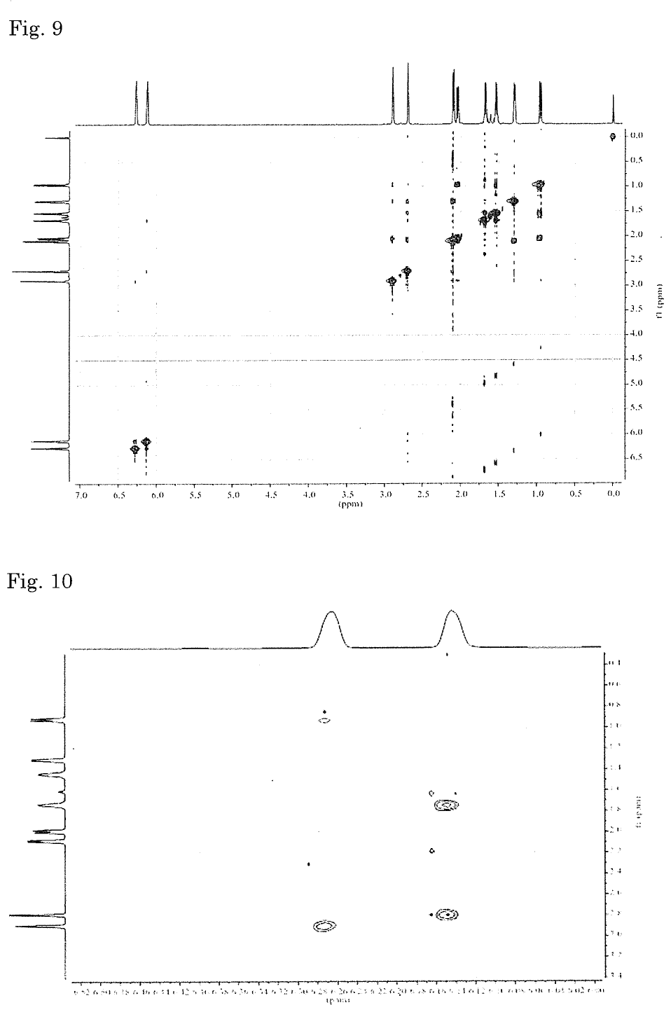

Here, FIGS. 2 to 5 show the results of the measurements on the peak (A-1) component by 1H-NMR, ¹³C-NMR, HSQC, and NOESY, respectively, and FIGS. 6 to 9 show the result of the measurements on the peak (A-2) component by ¹H-NMR, ¹³C-NMR, HSQC, and NOESY, respectively. Table 1 presents the attributions of the peak (A-1) component and the peak (A-2) component in ¹H-NMR and ¹³C-NMR. Note that together with the compounds (the compound represented by the (formula (X-1) and the compound represented by the formula (X-2)) in Table 1, the position numbers of proton confirmed are also presented.

TABLE 1

| | Peak (A-1) Component | Peak (A-2) Component | | |
|---|---|---|---|---|
| HPLC | 10.49 minutes | 10.49 minutes | | |
| Three-dimensional | cis-exo-exo | trans-exo-exo | | |
| Structure | | | | |
| Content | 28.20% | 56.45% | | |
| Three-dimensional Structure and NMR Data | 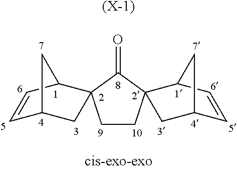 | 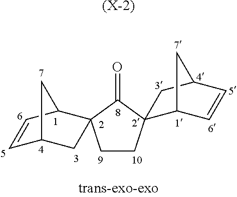 | | |
| Position | δC, ppm | δH, ppm | δC, ppm | δH, ppm |
| 1,1′ | 50.1 | 2.79 | 49.5 | 2.7 |
| 2,2′ | 55.4 | — | 55.3 | — |
| 3,3′ | 40.5 | 0.92, 1.99 | 40.2 | 0.96, 2.04 |
| 4,4′ | 42.8 | 2.89 | 42.8 | 2.89 |
| 5,5′ | 139.4 | 6.27 | 139.4 | 6.27 |
| 6,6′ | 134.5 | 6.14 | 134.8 | 6.13 |
| 7,7′ | 46.4 | 1.30, 2.09 | 46.4 | 1.30, 2.09 |
| 8 | 226.8 | — | 226.7 | — |
| 9 | 33.2 | 1.44, 1.74 | 33.3 | 1.53, 1.68 |
| 10 | 33.2 | 1.44, 1.74 | 33.3 | 1.53, 1.68 |

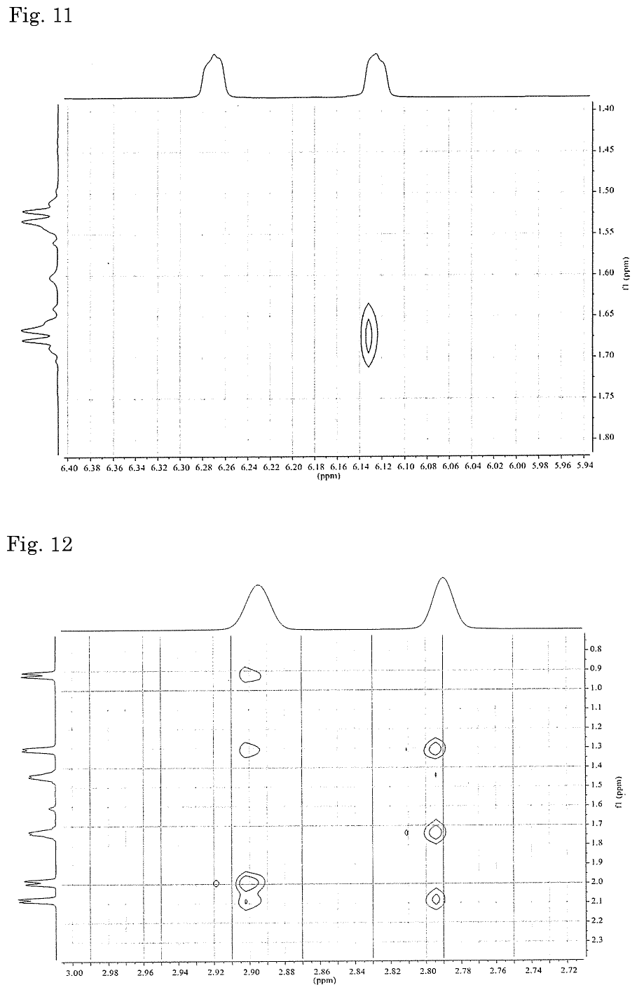

Note that for each of the peak (A-1) component and the peak (A-2) component, the three-dimensional configuration was confirmed by the NOESY method as follows. Specifically, since the correlation was observed between protons at positions 6, 6′ of the double bond (peak (A-1) component δH: 6.14 ppm; peak (A-2) component δH: 6.13 ppm) and protons at positions 9, 10 of the five-membered ring (peak (A-1) component δH: 1.74 ppm; peak (A-2) component δH: 1.68 ppm) in the portion of the norbornene ring in the NOESY spectrum, the peak (A-1) component and the peak (A-2) component in the peak (A) were confirmed such that the three-dimensional configuration of the carbonyl group (C═O) in the cyclopentanone ring relative to the norbornene ring in each of the peak (A-1) component and the peak (A-2) component was exo (the three-dimensional configuration was exo-exo). FIG. 10 and FIG. 11 show part of the NOESY spectrum on the peak (A-1) component and part of the NOESY spectrum on the peak (A-2) component, which were used in analyzing the three-dimensional configuration (exo or endo three-dimensional configuration) of the carbonyl group (C═O) in the cyclopentanone ring relative to the norbornene ring, respectively.

Here, since no correlation was observed between the proton at position 1 (δH: 2.79 ppm) and the protons at positions 3, 3′ (δH: 0.92 ppm and 1.99 ppm) in the NOESY spectrum, it was found that the two norbornane rings were arranged in cis relative to the cycloalkanone ring, and the peak (A-1) component was a cis isomer having spiro-fused rings. In this way, it was confirmed that the peak (A-1) component had a cis-exo-exo three-dimensional structure represented by the formula (X-1) in Table 1 from the NOESY spectrum. FIG. 12 shows part of the NOESY spectrum used in analyzing the cis or trans three-dimensional configuration of the two norbornane rings of the peak (A-1) component.

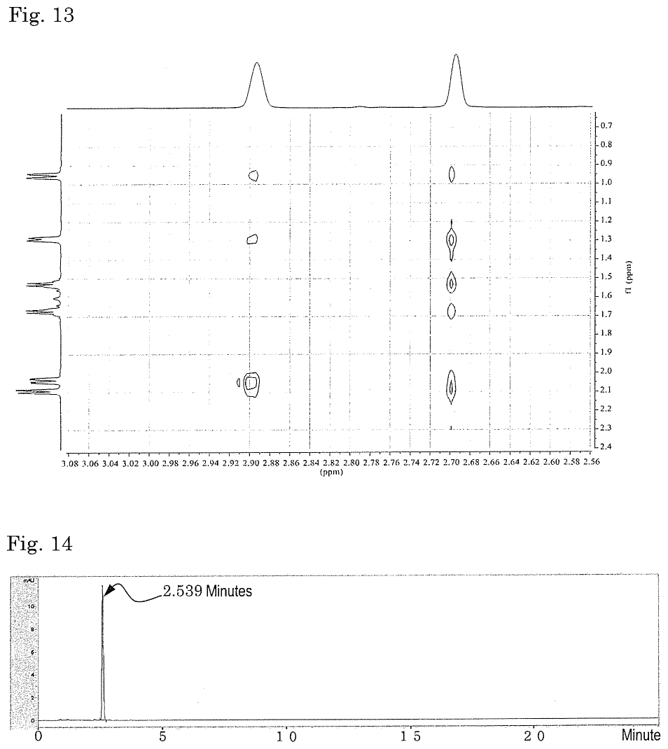

On the other hand, since the correlation was observed between proton at position 1 (δH: 2.70 ppm) and protons at positions 3, 3′ (δH: 0.96 ppm and δH: 2.04 ppm) in the NOESY spectrum, it was found that the two norbornane rings were arranged in trans relative to the cycloalkanone ring, and the peak (A-2) component was a trans isomer having spiro-fused rings. In this way, it was confirmed that the peak (A-2) component had a trans-exo-exo three-dimensional structure represented by the formula (X-2) in Table 1 from the NOESY spectrum. FIG. 13 show part of the NOESY spectrum used in analyzing the cis or trans three-dimensional configuration of the two norbornane rings of the peak (A-2) component.

Note that structural formulae of the compound having the cis-exo-exo three-dimensional structure represented by the formula (X-1) and the compound having the trans-exo-exo three-dimensional structure represented by the formula (X-2) are shown below.

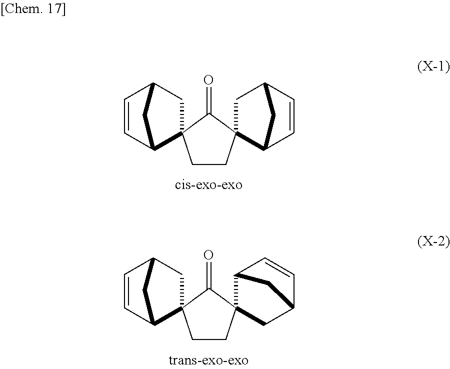

Note that as a result of analyzing the three-dimensional structures of the peak (B-1) component and the peak (B-2) component by the NOESY method, it was also confirmed that the peak (B-1) component was an isomer having a cis-exo-end three-dimensional structure and the peak (B-2) component was an isomer having a trans-exo-end three-dimensional structure.

From the result of such structural analysis, it was found that in the compound (product) obtained by Synthesis Example 1, the summed amount of the isomer having a cis-exo-exo three-dimensional structure (cis-exo-exo isomer) represented by the formula (X-1) and the isomer having a trans-exo-exo three-dimensional structure (trans-exo-exo isomer) represented by the formula (X-2) is 84.68% by mol relative to the summed amount of the stereoisomers and the content molar ratio between the cis-exo-exo isomer and the trans-exo-exo isomer is 1:2.

### Synthesis Example 2

First, methanol (820 mL), CuCl₂(II) (81.6 g, 454 mmol), the compound obtained in Synthesis Example 1 (35.6 g, 148 mmol), and Pd₃(OAc)₅(NO₂) (166 mg, 0.74 mmol in terms of Pd) were added into the container of a 1000 mL glass autoclave (manufactured by Taiatsu Techno Corporation under trade name of “Hyper Glaster TEM-V”) to obtain a mixture liquid. Note that Pd₃(OAc)₅(NO₂) was prepared by employing the method described in page 1991 of Dalton Trans (vol. 11) published in 2005.

Subsequently, a glass tube was placed so as to conduct bubbling of a gas in the mixture liquid present inside the container through the glass tube. Thereafter, the container was sealed and the atmosphere gas inside the container was replaced with nitrogen. Subsequently, a vacuum pump was connected to the container and the pressure inside the container was reduced. Next, the mixture liquid was stirred for 8 hours while carbon monoxide was supplied into the mixture liquid through the glass tube with bubbling to obtain a reaction liquid. Subsequently, an atmosphere gas containing carbon monoxide was removed from the inside of the container and the reaction liquid was concentrated with an evaporator to remove (distil away) methanol from the reaction liquid and to thus obtain a crude product (reaction product) (the obtained amount 69.6 g, yield 98.6%).

Next, toluene was added to the crude product (reaction product), followed by stirring vigorously for 1 hour to dissolve the crude product into the toluene to obtain a toluene solution. Thereafter, insolubles, which were not dissolved in toluene, were separated from the toluene solution through filtration to extract a product and to obtain a toluene extract. Subsequently, the toluene extract (filtrate) was washed twice with 5% by mass of hydrochloric acid and was then washed once with an aqueous solution of saturated sodium hydrogen carbonate. Then, the toluene extract after the washing was filtrated to obtain a filtrate. Next, the filtrate (toluene extract) thus obtained was heated to the boiling point (approximately 110° C.) of toluene to be concentrated to obtain a liquid concentrate. Thereafter, the liquid concentrate thus obtained was left to stand under a condition of room temperature (25° C.) to cool down for 12 hours. In this way, a solid component (white crystal) was precipitated in the liquid concentrate (crystallization step).

Subsequently, the solid component (white crystal) precipitated in the liquid concentrate was filtrated and separated, and then the solid component (white crystal) thus obtained was left to stand under a vacuum condition of 133 Pa or less for a whole day and night (24 hours), so that the solvent was evaporated and removed (dried) by a reduction in pressure to obtain a product (white crystal: 36.0 g, yield 51%). As a result of conducting IR measurement, NMR (¹H-NMR), and HPLC measurement in order to confirm the structure of the product (white crystal) thus obtained, it was confirmed that the product (white crystal) obtained was a compound (norbornane-2-spiro-a-cyclopentanone-α′-spiro-2″-norbornane-5,5″,6,6″-tetracarboxylic acid tetramethyl ester) represented by the following formula (XI):

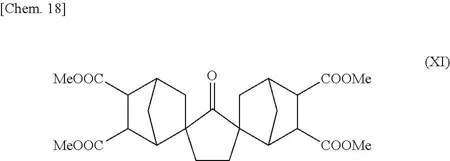 [SMILES: COC(=O)C1C2CC(C1C(=O)OC)C1(CCC3(CC4CC3C(C(=O)OC)C4C(=O)OC)C1=O)C2]

<Confirmation of Content Ratios of Stereoisomers in Compound Obtained in Synthesis Example 2>

In order to confirm the content ratios of the stereoisomers in the compound obtained in Synthesis Example 2, HPLC measurement was conducted on each of the compound thus obtained (crystallized white crystal) and a component (second product) that was not crystallized and remained in the filtrate (liquid concentrate) in the crystallization step to compare the HPLC spectra. Here, as a HPLC measurement method, the same HPLC measurement method (the same conditions) as the HPLC measurement method employed in confirming the content ratios of the stereoisomers in the compound obtained in Synthesis Example 1 was employed.

As a result of comparing the HPLC spectrum of the compound (crystallized white crystal) obtained in Synthesis Example 2 and the HPLC spectrum of the component (second product) that was not crystallized and remained in the filtrate (liquid concentrate) in the crystallization step of Synthesis Example 2, one peak was observed in the HPLC spectrum of the compound obtained in Synthesis Example 2, and from the peak position, it was confirmed that the compound obtained in Synthesis Example 2 was a component (a mixture of an isomer having a cis-exo-exo three-dimensional structure and an isomer having a trans-exo-exo three-dimensional structure) derived from a cis-exo-exo isomer and a trans-exo-exo isomer which were main components in the isomeric mixture of the compound obtained in Synthesis Example 1 (note that FIG. 14 shows the result of the HPLC measurement. In addition, the relation between the three-dimensional structure of the tetracarboxylic ester (tetraester) and the peak position of the HPLC spectrum was obtained by separately conducting HPLC measurement on the authentic sample of each isomer and then from the relation between the peak position of the HPLC spectrum of the authentic sample of each isomer and the peak position of the graph of the HPLC spectrum of the measurement target as described above). Note that regarding the esterification, while the three-dimensional structure of the raw material compound (the three-dimensional structure of each isomer in the compound obtained in Synthesis Example 1) is basically maintained, part of the cis-exo-exo isomer and the trans-exo-exo isomer remains in the filtrate at the time of crystallization; for this reason, the content molar ratios of the cis-exo-exo isomer and the trans-exo-exo isomer in the compound (crystallized white crystal) obtained in Synthesis Example 2 could be different from the content molar ratios of the cis-exo-exo isomer and the trans-exo-exo isomer in the compound obtained in Synthesis Example 1.

On the other hand, from the peak position of the HPLC spectrum, it was confirmed that the component (second product) that remained in the filtrate (liquid concentrate) was mainly a component derived from an exo-endo isomer which was an accessory component in the isomeric mixture of the compound obtained in Synthesis Example 1 (a mixture of the isomer having a cis-exo-endo three-dimensional structure and the isomer having a trans-exo-endo three-dimensional structure) (note that the relation between the three-dimensional structure of the tetracarboxylic ester (tetraester) and the peak position of the HPLC spectrum was obtained by separately conducting HPLC measurement on the authentic sample of each isomer and then from the relation between the peak position of the HPLC spectrum of the authentic sample of each isomer and the peak position of the graph of the HPLC spectrum of the measurement target as described above).

From the result of the HPLC measurement, it was found that the compound (crystallized white crystal) obtained in Synthesis Example 2 was composed of an isomeric mixture in which the total content of the isomer having the cis-exo-exo three-dimensional structure and the isomer having the trans-exo-exo three-dimensional structure was 100% by mol.

Note that J. Am. Chem. Soc., vol. 98, p. 1810, 1976 reported that in esterification, a methoxycarbonyl group is introduced to a bridgehead position (methylene head) in exo configuration while the three-dimensional structure of a raw material compound (norbornane) is maintained. From such a description of J. Am. Chem. Soc., vol. 98, p. 1810, 1976 as well, it is understood that in esterification as described above, the three-dimensional structure of the raw material compound (the three-dimensional structure of each isomer of the compound obtained in Synthesis Example 1) is maintained, and it is also understood that each methoxycarbonyl group in the white crystal obtained takes an exo three-dimensional configuration relative to the norbornane ring to which the methoxycarbonyl group is bonded.

### Example 1

First, into a 200 mL flask with a refluxing tube, a solution obtained by dissolving 24 g of the compound obtained in Synthesis Example 2 (the white crystal precipitated by the crystallization step: molecular weight 476.52) into 96 g of acetic acid was added. Thereafter, 0.19 g of trifluoromethanesulfonic acid (TfOH, boiling point: 162° C.) was added into the solution as a catalyst to obtain a reaction solution. Next, after the inside of the flask was replaced with nitrogen, the reaction solution was heated to 118° C. while being stirred under the condition of atmospheric pressure under the nitrogen gas stream. Subsequently, a step in which the reaction solution was refluxed at a temperature of 118° C. for 0.5 hours was conducted, and thereafter a step in which while the temperature was maintained at 118° C., a vapor (a mixture of methyl acetate and acetic acid) generated from the reaction solution was distilled away, and simultaneously acetic acid (acetic acid in an amount in which the solution was consumed by the distilling away of the vapor) was added into the flask by using the dropping funnel so that the amount of the solution in the flask was maintained constant (hereinafter the step is referred to simply as a “heating reaction step”) was conducted. One hour after the distilling away of the vapor (a mixture of methyl acetate, acetic acid, and water) was started by the heating reaction step, it was observed that a white precipitate was being generated in the solution (in the reaction solution) in the flask. Note that in the heating reaction step, the component (distillate) distilled out of the system was analyzed every 1 hour through mass measurement and gas chromatograph to check the degree of progress of the reaction. Note that it was confirmed from the analysis that acetic acid, methyl acetate, and water were present in the distillate. Eight hours after the distilling away of the vapor (the mixture of methyl acetate, acetic acid, and water) was started in the heating reaction step, it was confirmed that the distillation of methyl acetate was stopped, and accordingly, the heating reaction step was ended by stopping the heating. Note that the amount of methyl acetate distilled (except for scattered methyl acetate) from the start of distilling away to after 8 hours was 13.2 g (88%). After the heating reaction step was ended (after the heating was stopped), the reaction solution (the solution in which the white precipitate was generated) inside the flask was left to stand for a whole day and night (24 hours) at room temperature, and then filtration under reduced pressure using a paper filter was conducted to separate a white solid component from the reaction solution. Subsequently, the white solid component thus obtained was washed with ethyl acetate, and thereafter, a step of drying under reduced pressure was conducted to obtain a product composed of 17.6 g (yield 91%) of a white powder.

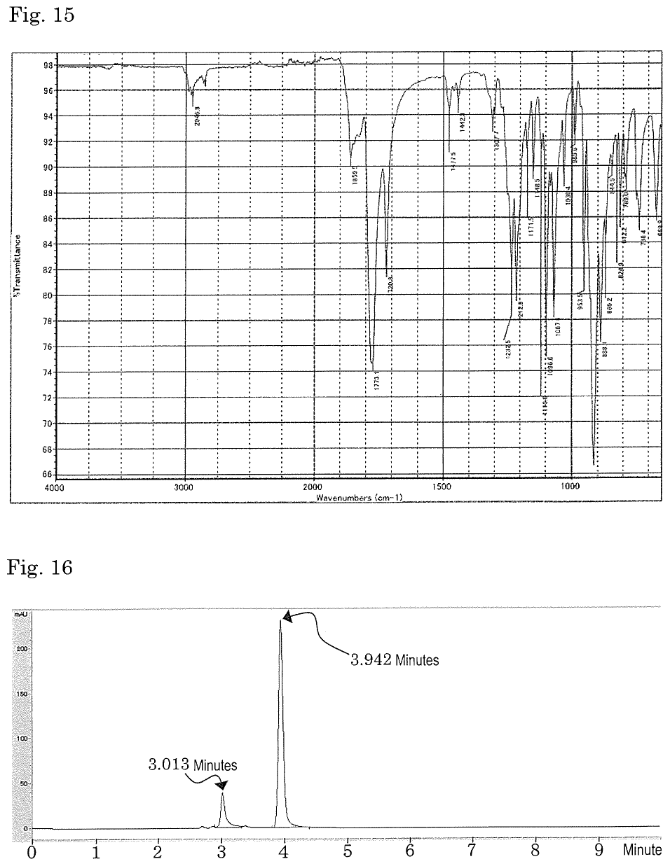

In order to confirm the structure of the product thus obtained, IR measurement was conducted. As a result of the IR measurement, FIG. 15 shows the IR spectrum of the white powder thus obtained. As is clear from the result shown in FIG. 15 as well, it was confirmed that the white powder thus obtained was norbornane-2-spiro-a-cyclopentanone-α′-spiro-2″-norbornane-5,5″,6,6″-tetracarboxylic dianhydride (CpODA: a compound represented by the formula (A)). Note that hereinafter, the CpODA obtained in Example 1 is sometimes referred to simply as a “CpODA-(A)”.

<Confirmation of Content Ratios of Stereoisomers in Compound (CpODA) Obtained in Example 1>

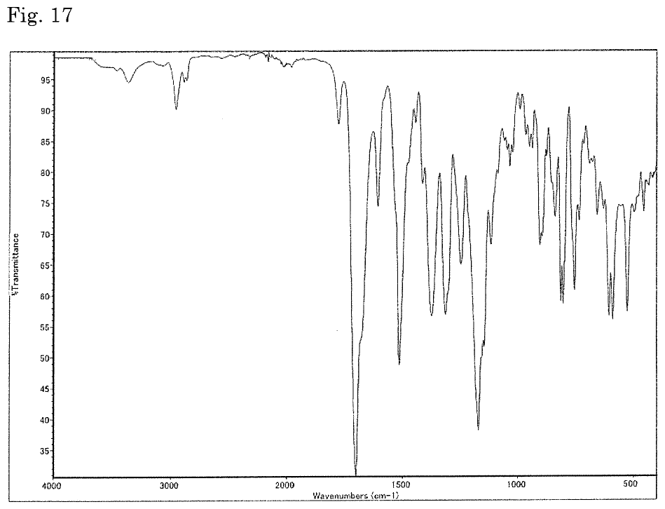

In order to confirm the ratios of the isomers in the compound (CpODA) obtained in Example 1, HPLC measurement as follows was conducted. Specifically, trade name “1200 Series” manufactured by Agilent Technologies was used as a measurement device, trade name “ZORBAX SB-CN” manufactured by Agilent Technologies was used as a column, a mixture of n-hexane and 1,4-dioxane (n-hexane/1,4-dioxane=40 mL/60 mL) was used as a solvent, the flow speed of the solvent was set to 1.0 mL/min., the detection wavelength of the diode array detector (DAD) was set to 230 nm, the temperature was set to 35° C., a sample obtained by adding 1 mg of the product (crystal) to 1.5 mL of the solvent was prepared, and then the HPLC measurement was conducted. Note that the ratios of the isomers were obtained from the area ratios of the HPLC by using a calibration curve (reference sample: naphthalene). FIG. 16 shows the result of the HPLC measurement. As is clear from the result of the HPLC measurement (the result of the HPLC spectrum shown in FIG. 16), peaks (two peaks) were confirmed at the position of 3.01 minutes and the position of 3.94 minutes, respectively, and it was confirmed that the compound (CpODA) obtained in Example 1 was a mixture of two isomers. Here, as is clear from the description in page 1117 of Macromolecules (vol. 27) published on 1994, since the three-dimensional configuration of a raw material compound is maintained in a transesterification reaction using an acid catalyst, it is understood that in the compound obtained, the three-dimensional structure of each isomer contained in the compound obtained in Synthesis Example 2 is maintained. Then, from the result of the measurement and the like, it was also found that the compound (CpODA) obtained in Example 1 was a mixture of an isomer having a cis-exo-exo three-dimensional structure and an isomer having a trans-exo-exo three-dimensional structure (an isomeric mixture in which the summed content ratio of the cis-exo-exo isomer and the trans-exo-exo isomer is 100% by mol). Note that the relation between the three-dimensional structure of the tetracarboxylic dianhydride (CpODA) and the peak position of the HPLC spectrum was obtained by separately conducting HPLC measurement on the authentic sample of each isomer (the authentic sample of each isomer of the CpODA) and then from the relation between the peak position of the HPLC spectrum of the authentic sample and the peak position of the graph of the HPLC spectrum of the measurement target as described above. Then, upon confirming the ratio of each isomer based on the area ratio of the peak of the HPLC measurement, it was also found that the CpODA obtained in Example 1 was such that the content of the trans-exo-exo isomer was 84.2% by mol and the content of the cis-exo-exo isomer was 15.8% by mol.

### Example 2

A CpODA (the compound represented by the formula (A) ) was synthesized by using the same method as the method employed in Example 2 of WO 2014/050788 except that the compound obtained in Synthesis Example 2 (white crystal precipitated by the crystallization step: molecular weight 476.52) was used as a norbornane tetracarboxylic acid tetramethyl ester and the step of scraping a crystal (solid component) adhering to the container with a tweezers or a medicine spoon when the container containing the catalyst was taken out was not conducted.

Specifically, first, a woven fabric of polyphenylene sulfide (PPS) (mesh cloth manufactured by NBC Meshtec inc. under the trade name of “PPSP60”) was prepared as a porous fabric, and a cylindrical body (envelope-shaped) having no gussets and having opening portions on both sides that takes on a cylindrical shape having a diameter of 20 mm and a length of 46 mm when the opening portions are opened was formed by using the porous fabric. Subsequently, 2.5 g of an acidic ion exchange resin (manufactured by Organo Corporation under the trade name of “Amberlite 200CT”, average particle size: 0.60 to 0.85 mm) ([raw material compound (mole)]:[catalyst (molar amount in terms of functional group)]=1:0.3) was prepared as a catalyst, and the catalyst was placed near the center of the inside of the cylindrical body (envelope-shaped). Thereafter, an envelope-shaped mesh bag was formed by sewing the opening portions on the opposite sides of the cylindrical body (envelope-shaped) with a multifilament yarn made of the same material (PPS) as that of the fabric. In this way, a container that was made of the porous fabric and contained the catalyst made of the ion-exchange resin (hereinafter referred to as a “catalyst-packed bag”) was prepared (note that the same catalyst-packed bag as that used in Example 2 of WO 2014/050788 in terms of the specific shape of the container, the amount of the catalyst, the bulk ratio of the ion-exchange resin, and the like was prepared).

Subsequently, a solution obtained by dissolving 10.0 g of the compound (the white crystal precipitated by the crystallization step: norbornane tetracarboxylic acid tetramethyl ester) obtained in Synthesis Example 2 into 165 g of acetic acid was put into a flask of a volume of 200 mL with a refluxing tube, and further the catalyst-packed bag obtained as described above was hanged in the flask by means of a string made of the same material as that of the bag and was immersed in the solution. Next, the solution in the flask was heated such that the temperature in the flask reached 115° C. while being stirred, and was refluxed for 0.5 hours. After the reflux step, a reaction step in which a vapor generated was distilled away by using a Liebig condenser under a heating condition of 115° C., and simultaneously, acetic acid was added into the flask by using a dropping funnel so that the amount of the solution in the flask was maintained constant was conducted continually for 20 hours after the distilling away of the vapor was started. Note that when the reaction step was being conducted, the distillate distilled away was sampled every certain time, and was analyzed through weight measurement and gas chromatograph, from which the presence of acetic acid, methyl acetate, and water was observed in the distillate. In addition, in the reaction step, it was confirmed that from around 6 hours from the start of the distillation, a white precipitate was generated in the solution in the flask. After 20 hours from the start of the distilling away of the vapor as described above, the reaction step was ended by stopping the heating, and the catalyst-packed bag was taken out of the flask (note that a precipitated component adhering to the catalyst-packed bag, but was used in Example 3 described later in the state of being taken out). Thereafter, the white crystal was further precipitated by distilling away acetic acid from the solution in the flask to obtain a liquid concentrate. Next, filtration under reduced pressure was conducted on the liquid concentrate by using a Kiriyama funnel to obtain a white solid component. The white solid component thus obtained was washed with toluene and then dried to obtain a product made of 6.0 g (yield: 74%) of a white powder. The product (white powder) thus obtained was found to be CpODA by confirmation through IR and ¹H-NMR. Note that hereinafter, the CpODA obtained in Example 2 is sometimes referred to simply as the “CpODA-(B)”.

<Confirmation of Content Ratios of Stereoisomers in Compound (CpODA) Obtained in Example 2>

In order to confirm the content ratios of the stereoisomers in the compound (CpODA) obtained in Example 2, the same method as the method for confirming the content ratios of the stereoisomers in the compound (CpODA) obtained in the Example 1 was employed to conduct HPLC measurement, from which it was found that the compound (CpODA) obtained in Example 2 was a mixture of an isomer having a cis-exo-exo three-dimensional structure and an isomer having a trans-exo-exo three-dimensional structure (an isomeric mixture in which the summed content ratio of the cis-exo-exo isomer and the trans-exo-exo isomer was 100% by mol). Upon confirming the ratios of the isomers based on the area ratios of peaks of the HPLC measurement in the above-described manner, it was also found that the content of the trans-exo-exo isomer was 95.8% by mol and the content of the cis-exo-exo isomer was 4.2% by mol.

### Example 3

The product was collected as follows by using the catalyst-packed bag taken out after the reaction step was ended in Example 2. Specifically, the catalyst-packed bag taken out after the reaction step was ended was opened, and was immersed into acetic acid (200 mL) to separate the white solid component adhering to the catalyst-packed bag and the ion-exchange resin from the ion-exchange resin and the catalyst-packed bag, followed by influxing to dissolve the white solid component into the acetic acid, so that a solution was obtained. Subsequently, by using a Kiriyama funnel, hot filtration was conducted such that a crystal was not precipitated on the funnel, to remove an ion-exchange resin from the solution, collecting a filtrate. Thereafter, the filtrate thus obtained was concentrated to obtain a white solid component. Subsequently, the white solid component thus obtained was washed with toluene and then dried to obtain a product made of 1.9 g (yield: 24%) of a white powder. Upon confirming the product (white powder) thus obtained through IR and ¹H-NMR, the product (white powder) was found to be a CpODA. Note that hereinafter the CpODA obtained in Example 3 was sometimes referred to simply as a “CpODA-(C)”.

<Confirmation of Content Ratios of Stereoisomers in Compound (CpODA) Obtained in Example 3>

In order to confirm the content ratios of the stereoisomers in the compound (CpODA) obtained in Example 3, the same method as the method for confirming the content ratios of the stereoisomers in the compound (CpODA) obtained in Example 1 was employed to conduct HPLC measurement, from which it was found that the compound (CpODA) obtained in Example 2 was a mixture of an isomer having a cis-exo-exo three-dimensional structure and an isomer having a trans-exo-exo three-dimensional structure (an isomeric mixture in which the summed content ratio of the cis-exo-exo isomer and the trans-exo-exo isomer was 100% by mol). Moreover, upon confirming the ratios of the isomers based on the area ratios of peaks of the HPLC measurement in the above-described manner, it was also found that the content of the trans-exo-exo isomer was 48.8% by mol and the content of the cis-exo-exo isomer was 51.2% by mol.

### Comparative Example 1

A CpODA for comparison was prepared by conducting the following first to third steps in the same manner as that in the method employed in Synthesis Example 1, Example 1, and Example 2 described in WO 2011/099518.

<First Step>

First, 6.83 g of a 50% by mass aqueous solution of dimethylamine (dimethylamine: 75.9 mmol) was added into a 100 mL two-necked flask. Next, 8.19 g of a 35% by mass aqueous solution of hydrochloric acid (hydrogen chloride: 78.9 mmol) was added into a 100 mL dropping funnel. Subsequently, the dropping funnel was set to the two-necked flask, and the aqueous solution of hydrochloric acid was dropped into the aqueous solution of dimethylamine under ice cooling to prepare dimethylamine hydrochloride in the two-necked flask. Next, 2.78 g of paraformaldehyde (92.4 mmol) and 2.59 g of cyclopentanone (30.8 mmol) were further added into the two-necked flask. Subsequently, a bulb condenser was set to the two-necked flask, and then the inside of the two-necked flask was replaced with nitrogen. Thereafter, the two-necked flask was immersed into an oil bath at 90° C., followed by heating and stirring for 3 hours to obtain a reaction liquid. Next, the reaction liquid in the two-necked flask was cooled down to 50° C., and then methyl cellosolve (50 mL), 1.12 g of a 50% by mass aqueous solution of dimethylamine (12.4 mmol), and 7.13 g of cyclopentadiene (108 mmol) were added to the reaction liquid to obtain a mixture liquid. Subsequently, the inside of the two-necked flask was purged with nitrogen, the two-necked flask was then immersed into an oil bath at 120° C. to heat the mixture liquid for 90 minutes, and then the mixture liquid was cooled down to room temperature (25° C.) Next, the mixture liquid was transferred into a 200 mL separating funnel, n-heptane (80 mL) was added to the mixture liquid, and then the n-heptane layer was collected from the mixture liquid, thereby conducting the first extraction operation. Next, after the n-heptane layer was collected from the mixture liquid, n-heptane (40 mL) was again added to the remaining methyl cellosolve layer, and n-heptane layer was collected, thereby conducting the second extraction operation. Then, the n-heptane layers obtained by the first and second extraction operations were mixed to obtain an n-heptane extract. Next, the n-heptane extract was washed once with 5% by mass NaOH water (25 mL), and then was washed once with 5% by mass hydrochloric acid water (25 mL). Subsequently, the n-heptane extract washed with the hydrochloric acid water was washed once with 5% sodium bicarbonate water (25 mL), and then washed once with saturated saline water (25 mL). The n-heptane extract washed in this way was dried with anhydrous magnesium sulfate, and anhydrous magnesium sulfate was filtrated and removed to obtain a filtrate. Subsequently, the filtrate thus obtained was concentrated with an evaporator, and n-heptane was distilled away to obtain 7.4 g of a crude product (gross yield 99%). Next, the Kugelrohr distillation (boiling point: 105° C./0.1 mmHg) was conducted on the crude product thus obtained to obtain 4.5 g of a product (yield 61%).

In order to confirm the structure of the product thus obtained, IR and NMR (¹H-NMR and ¹³C-NMR) measurements were conducted, and it was confirmed that the product obtained was 5-norbornene-2-spiro-2′-cyclopentanone-5′-spiro-2″-5″-norbornene.

<Second Step>

The 5-norbornene-2-spiro-2′-cyclopentanone-5′-spiro-2″-5″-norbornene (2.00 g, 8.32 mmol) obtained in the first step, methanol (800 ml), sodium acetate (7.52 g, 91.67 mmol), CuCl₂(II) (8.95 g, 66.57 mmol), and PdCl₂ (34 mg, 0.19 mmol) were set in a 2 L four-necked flask to obtain a mixture liquid, and then the atmosphere inside the flask was purged with nitrogen. Next, the mixture liquid was vigorously stirred for 1 hour under conditions of 25° C. and 0.1 MPa while carbon monoxide (3.2 L) was introduced into the flask by using a balloon to obtain a reaction liquid. Subsequently, carbon monoxide was removed from the inside of the flask, the reaction liquid was concentrated with an evaporator, and methanol was completely removed from the reaction liquid to obtain a reaction product. Thereafter, chloroform (500 ml) was added to the reaction product, followed by filtration through celite, and then the filtrate was separated with an aqueous solution of saturated sodium hydrogen carbonate to collect an organic layer. Then, a drying agent (anhydrous magnesium sulfate) was added to the organic layer, followed by stirring for 2 hours. Subsequently, the drying agent was filtrated and removed from the organic layer, and the organic layer was concentrated with an evaporator to obtain a product (obtained amount 3.93 g, yield 99.1%).

In order to confirm the structure of the product thus obtained, IR and NMR measurements were conducted, and it was confirmed that the product obtained was norbornane-2-spiro-a-cyclopentanone-α′-spiro-2″-norbornane-5,5″,6,6″-tetracarboxylic acid tetramethyl ester (a compound represented by the formula (XI)).

<Third Step>

The norbornane-2-spiro-a-cyclopentanone-α′-spiro-2″-norbornane-5,5″,6,6″-tetracarboxylic acid tetramethyl ester (1.93 g, 4.05 mmol) obtained in the second step, formic acid (14 ml, 222 mmol), and p-toluenesulfonic acid (anhydride, 0.1 g, 0.306 mmol) were set in a 100 ml three-necked flask and heating under reflux was conducted in an oil bath at 120° C. for 6 hours to obtain a mixture liquid. Subsequently, the mixture liquid was concentrated through distillation under reduced pressure such that the amount of liquid in the mixture liquid became approximately half, to obtain a liquid concentrate. Thereafter, formic acid (7 ml, 111 mmol) was added to the liquid concentrate, followed by heating under reflux at 120° C. for 6 hours, and then the mixture liquid thus obtained was concentrated again through distillation under reduced pressure such that the amount of liquid in the mixture liquid became approximately half, to obtain a liquid concentrate. Then, the operations of addition of formic acid and concentration on the liquid concentrate were further repeated three times in total, and then formic acid (7 ml, 111 mmol) and acetic anhydride (18 ml, 127 mmol) were added to the liquid concentrate thus obtained, followed by heating under reflux at 120° C. for 3 hours to obtain a reaction liquid. Then, the reaction liquid thus obtained was concentrated with an evaporator to be dried and solidified to obtain a solid component. Next, diethyl ether was added to the solid component thus obtained to wash the solid component to obtain a gray crude product (1.56 g, quantitative). Subsequently, the crude product (1.0 g) thus obtained was placed inside a sublimation purifier and was purified by sublimation at 250 to 270° C./1 mmHg over three and a half hours to obtain 0.89 g (yield 89.1%) of a product (white solid) In order to confirm the structure of the product thus obtained, IR and NMR measurements were conducted, and it was confirmed that the product was a CpODA. Note that hereinafter the CpODA obtained in Comparative Example 1 is sometimes referred to simply as a “CpODA-(D)”.

<Confirmation of Content Ratios of Stereoisomers in Compound (CpODA) Obtained in Comparative Example 1>

In order to confirm the content ratios of the stereoisomers in the compound (CpODA) obtained in Comparative Example 1, the same method as the method for confirming the content ratios of the stereoisomers in the compound (CpODA) obtained in Example 1 was employed to conduct HPLC measurement, from which it was found that the compound (CpODA) obtained in Comparative Example 1 was a mixture of 6 isomers. Upon confirming the ratios of the isomers based on the area ratios of peaks of the HPLC measurement in the above-described manner, it was found that the content of the trans-exo-exo isomer was 0.4% by mol, the content of the cis-exo-exo isomer was 0.4% by mol, the content of the trans-endo-endo isomer was 49.8% by mol, the content of the cis-endo-endo isomer was 29.2% by mol, the content of the trans-exo-endo isomer was 10.1% by mol, and the content of the cis-exo-endo isomer was 10.1% by mol.

Note that Table 2 below presents the ratios of the contents of the isomers contained in the CpODA-(A) to (D). In addition, the purities of the CpODAs in the products produced in Examples 1 to 3 and Comparative Example 1 are also presented together in Table 2. The purities were obtained by conducting HPLC measurement under the same conditions as those in the HPLC measurement employed as the method for confirming the content ratios of the stereoisomers in Synthesis Example 1 and Synthesis Example 2. Here, the purity (%) was calculated in area percentage of the peak.

In addition, the contents of components other than the CpODAs, which can be contained in the products produced in Examples 1 to 3 and Comparative Example 1, and the like were analyzed, and the result thus obtained is presented together in Table 2. Note that components other than the CpODAs, which can be contained in the products, include unreacted raw material (ester compounds represented by the above-described formula (XI)); halfesters (reaction intermediates) represented by the following formula (Y):

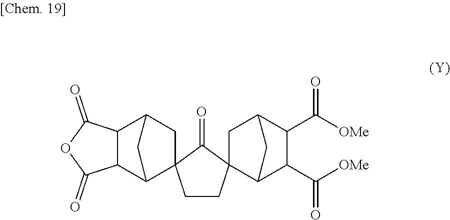 [SMILES: COC(=O)C1C2CC(C1C(=O)OC)C1(CCC3(CC4CC3C3C(=O)OC(=O)C43)C1=O)C2]

tetracarboxylic acids (reaction intermediates) in which groups represented by a formula: —COOMe in ester compounds represented by the above-described formula (XI) are all replaced with groups represented by a formula: —COOH; heavy substances (by-products); halogen; sulfur; metal components (B, Cu, Pd, Na, K, and the like); and the like. The contents of components other than the CpODAs were analyzed as described below.

The residual ratio (% by area) of the ester compound represented by the formula (XI) and the halfester represented by the formula (Y) were analyzed by dissolving the product obtained in each Example and the like into deuterated DMSO (deuterated dimethylsulfoxide) and conducting 1H-NMR measurement at an NMR of 600 MHz. Note that as the residual ratio, the ratio (area ratio) to the integral value of the reference signal (1.14 ppm) of the CpODA analyzed by NMR based on the integral value of the signal (3.52 ppm) of the methyl group of the remaining methyl ester was calculated.

In addition, the content ratios (% by area) of heavy substances were measured by using “TOSOH HLC-8220GPC” manufactured by Tosoh corporation as a measurement device, and further using a column obtained by coupling one column of “TOSOH TSKguardcolumn SuperMP(HZ)-M” manufactured by Tosoh corporation and three columns of “TOSOH TSKgelSuperMultiporeHZ-M” manufactured by Tosoh corporation. In addition, in the measurement, a differential refractive index detector (RI detector) was used as a detector, and tetrahydrofuran (THF) was used as a solvent. In addition, in the measurement, 2 mg of each product was used as a sample. Then, a measurement sample obtained by dissolving 2 mg of the sample into 1.5 mL of THF was prepared, and GPC measurement was conducted under conditions of flow speed: 0.35 ml/min. and injection volume: 5 μl. Since the peak appeared before the peak of CpODA was the peak of the heavy substances (by-products), the content ratio of the heavy substances was calculated in area percentage.

In addition, the content ratios of other components such as halogen and metal components (trace metals) were commissioned to an outside analysis center and were obtained as follows. Specifically, the contents (ppm) of halogen and sulfur were obtained through the combustion-ion chromatography, and the content ratios (ppm) of metal components such as B, Cu, Pd, Na, and K were obtained by analysis with the ICP method. In addition, the content (% by area) of tetracarboxylic acid, which was the reaction intermediate, was measured by the liquid chromatography tandem mass spectrometry (LC/MS/MS).

Moreover, the transmittance (%) of light of 400 nm was measured on the CpODAs generated in Examples 1 to 3 and Comparative Example 1. Specifically, first each of the CpODAs generated in Examples 1 to 3 and Comparative Example 1 was used, and N,N-dimethylacetamide (DMAc) was used as a solvent, and the CpODA (200 mg) was measured and taken into a 30 ml sample vial with a lid for each CpODA, and 3800 mg of DMAc was added thereto. Subsequently, an ultrasonic wave was being applied until it was visually observed that the CpODA was dissolved into DMAc and the solution became uniform to prepare a solution (solvent: DMAc) of each CpODA with a concentration of 5% by mass. Next, the solution after the solution became uniform (the CpODA was dissolved) was visually observed (note that in Table 2, the solution is referred to as a “5% solution of DMAc solvent”) was used as a measurement sample and was placed in a quartz cell having an optical path length of 10 mm. Then, the transmittance (%) of the measurement sample for light of 400 nm was measured by using a spectrophotometer to obtain the transmittance (%) of each CpODA for the light of 400 nm. Note that the reference used for the measurement of transmittance as described above was N,N-dimethylacetamide (DMAc). The result thus obtained is presented together in Table 2.

TABLE 2

| | | | | | Comparative |
|---|---|---|---|---|---|
| | | Example 1 | Example 2 | Example 3 | Example 1 |
| Type of CpODA | | CpODA-(A) | CpODA-(B) | CpODA-(C) | CpODA-(D) |
| Ratios of | trans-exo-exo | 84.2 | 95.8 | 48.8 | 0.4 |
| Isomers | Isomer | | | | |
| in CpODA | cis-exo-exo | 15.8 | 4.2 | 51.2 | 0.4 |
| (% by mol) | Isomer | | | | |
| | trans-endo-endo | — | — | — | 49.8 |
| | Isomer | | | | |
| | cis-endo-endo | — | — | — | 29.2 |
| | Isomer | | | | |
| | trans-exo-endo | — | — | — | 10.1 |
| | Isomer | | | | |
| | cis-exo-endo | — | — | — | 10.1 |
| | Isomer | | | | |
| | Summed Amount | 100 | 100 | 100 | 100 |
| | of Isomers | | | | |

| | | | | |
|---|---|---|---|---|
| Purity of CpODA (%) | 99.74 | 99.55 | 99.64 | 98.63 |
| [Measurement Method: HPLC Method] | | | | |
| Heavy Substances (%) | 0.05 | 0.17 | 0.16 | 0 |
| [Measurement Method: GPC Method] | | | | |
| Residual Ratios of Halfester and Ester | 0.17 | 0.18 | 0.19 | 1.36 |
| Compound (%) | | | | |
| [Measurement Method: NMR Method] | | | | |
| B Content (ppm) | <0.2 | <0.2 | <0.2 | — |
| [Measurement Method: ICP Method] | | | | |
| Cu Content (ppm) | <0.2 | <0.2 | <0.2 | <2 |
| [Measurement Method: ICP Method] | | | | |
| Pd Content (ppm) | <0.2 | <0.2 | <0.2 | <2 |
| [Measurement Method: ICP Method] | | | | |
| Na Content (ppm) | <1 | <1 | <1 | <2 |
| [Measurement Method: ICP Method] | | | | |
| K Content (ppm) | <0.2 | <0.2 | <0.2 | <2 |
| [Measurement Method: ICP Method] | | | | |
| Ca Content (ppm) | <0.01 | <0.01 | <0.01 | — |
| [Measurement Method: ICP Method] | | | | |
| Fe Content (ppm) | <0.1 | <0.1 | <0.1 | 19 |
| [Measurement Method: ICP Method] | | | | |
| Cl Content (ppm) | 6 | 13 | 13 | 2 |
| [Measurement Method: Combustion-ion | | | | |
| Chromatography] | | | | |
| S Content (ppm) | <2 | <2 | <2 | <2 |
| [Measurement Method: Combustion-ion | | | | |
| Chromatography] | | | | |
| Content of Tetracarboxylic Acid (%) | 0.04 | 0.10 | 0.01 | 0.01 |
| [LC-MS Method] | | | | |
| 400 nm Transmittance of CpODA (%) | 99.6 | 99.4 | 99.3 | 90.0 |
| [Measurement Sample: 5% Solution | | | | |
| of DMAc Solvent Measurement Method: | | | | |
| UV-Vis Method] | | | | |

### [Preparation of Polyimide]

Here, first, the method (measuring method) for evaluating the properties of a polyimide obtained in each of Examples and Comparative Examples and the like, which will be described later, will be described. Note that regarding the polyamic acids obtained in Examples and Comparative Examples, the intrinsic viscosity [η] was measured, and regarding the polyimides (films) obtained in Examples and Comparative Examples, linear expansion coefficient (CTE), glass transition temperature (Tg), total luminous transmittance, yellowness index (YI), dielectric dissipation factor (tan δ), and relative permittivity (εr) were measured. Note that for the IR measurement of the polyimides, IR measurement devices (manufactured by JASCO corporation under the trade name of FT/IR-460, FT/IR-4100) were used.

<Method for Measuring Coefficient of Thermal Expansion (CTE)>

The linear expansion coefficient was calculated as follows. A film having a size of 20 mm in length and 5 mm in width was cut out from the polyimide (film) obtained in each Example or the like as a measurement sample. A change in length of the sample from 50° C. to 200° C. was measured by employing conditions of tension mode (49 mN) and a rate of temperature rise of 5° c./min. under a nitrogen atmosphere by using a thermomechanical analyzer (manufactured by Rigaku corporation under the trade name of “TMA8311”) as a measurement device. Then, an average value of the change in length per 1° C. was obtained from the change in length in a temperature range of 100° C. to 200° C.

<Method for Measuring Glass Transition Temperature (Tg)>

The glass transition temperature (unit: ° C.) was obtained as follows. A film having a size of 20 mm in length and 5 mm in width was cut out from the polyimide (film) obtained in each Example or the like as a measurement sample (the thickness of the sample was not changed from the thickness of the film obtained in each Example or the like). A TMA curve was obtained by conducting measurement under conditions of tension mode (49 mN) and a rate of temperature rise of 5° c./min. under a nitrogen atmosphere by using a thermomechanical analyzer (manufactured by Rigaku corporation under the trade name of “TMA8311”) as a measurement device. The value of the glass transition temperature (Tg) (unit: ° C.) of the resin forming the film obtained in each Example or the like was obtained by extraporating a curve across an inflection point of the TMA curve attributable to glass transition.

<Method for Measuring Total Luminous Transmittance>

The value of the total luminous transmittance (unit: %) was obtained by conducting measurement according to JIS K7361-1 (published in 1997) using the polyimide (film) obtained in each Example as a sample for measurement as it is and using trade name “Haze Meter NDH-5000” manufactured by Nippon Denshoku Industries Co., Ltd. as a measurement device.

<Method for Measuring Yellowness Index (YI) and Transmittance for Light with Wavelength of 400 nm>

The value of the yellowness index (YI) was obtained by conducting measurement according to ASTM E313-05 (published in 2005) by using the polyimide (film) obtained in each Example or the like as a sample for measurement as it is and using trade name “Spectrophotometer SD6000” manufactured by Nippon Denshoku Industries Co., Ltd. as a measurement device. In addition, the value of the transmittance for light with a wavelength of 400 nm was obtained by employing the same measuring method as the measuring method for the value of YI on the polyimide (film) obtained in each Example or the like.

<Method for Measuring Dielectric Dissipation Factor (tan δ) and Relative Permittivity (εr)>

The dielectric dissipation factor (tan δ) and the relative permittivity (εr) were measured as follows by forming a sample piece having a size of 52 mm in width and 76 mm in length from the polyimide (film) obtained in each Example or the like and employing the split post dielectric (SPDR) resonator method. Specifically, the values of the dielectric dissipation factor (tan δ) and the relative permittivity (εr) were measured in a laboratory the environment of which was adjusted to 23° C. and a relative humidity of 50% after a test piece (width: 52 mm, length: 76 mm, and film thickness: 10 μm) formed as described above was dried at 85° C. for 2 hours. In addition, trade name “Vector Network Analyzer PNA-X N5247A” manufactured by KeySight Technologies (former Agilent Technologies) was used as a measurement device. In addition, in the measurement, the test piece was set in the SPDR dielectric resonator of the measurement device, and actual measured values of the dielectric dissipation factor (tan δ) and the relative permittivity (εr) were obtained with a frequency of 10 GHz. Then, the measurement of the actual measured value described above was conducted four times in total, and the values of the dielectric dissipation factor (tan δ and the relative permittivity (εr) were obtained by obtaining average values of these measured values. In this way, as the values of the dielectric dissipation factor (tan δ) and the relative permittivity (εr), the average values of the actual measured values obtained by the four times of measurement were employed.

<Measurement of Intrinsic Viscosity [η]>

The intrinsic viscosity [η] of the polyamic acid formed in the reaction liquid to be used in producing a film or the like in each Example or the like was measured by preparing a measurement sample of the polyamic acid with a concentration of 0.5 g/dL using N,N-dimethylacetamide as a solvent from the reaction liquid and using an automatic viscometer (trade name “VMC-252”) manufactured by RIGO CO., LTD as a measurement device under a temperature condition of 30° C.

<Measurement of 5% Weight Loss Temperature (Td5%)>

The 5% weight loss temperature of the polyimide (film) obtained in each of Examples 23 to 39 was measured as follows. Specifically, first, a sample with 2 to 4 mg was prepared from the polyimide (film) obtained in each Example, and the sample was placed in an aluminum-made sample pan, a thermogravimetric analyzer (manufactured by SII NanoTechnology Inc. under the trade name of “TG/DTA7200”) was used as a measurement device, the sample was heated under conditions of a scanning temperature set to from 30° C. to 550° C. and a rate of temperature rise of 10° c./min. under a nitrogen gas atmosphere, and the temperature at which the weight of the sample was reduced by 5% was measured. Note that although the 5% weight loss temperature was measured also for the polyimide (film) obtained in Example 4, the method for measuring the 5% weight loss temperature of the polyimide (film) obtained in Example 4 will be described separately. In addition, although the methods for measuring the 5% weight loss temperatures of the polyimide (film) obtained in Example 4 and the polyimide (film) obtained in Examples 23 to 39 are different in the range of the scanning temperature and the like, it is obvious that the measured values basically take the same value by either of the measuring methods.

<Measurements of Tensile Elastic Modulus and Fracture Elongation>

The tensile elastic modulus (unit: GPa) and the fracture elongation (unit: %) of the polyimide (film) obtained in each Example or the like were measured as follows. Specifically, in these measurements, first, trade name “Super Dumbbell Cutter manufactured by Dumbbell Co., Ltd (model: SDMK-1000-D, according to A22 standard of JIS K7139 (published in 2009) )” was attached to SD Lever Type Sample Cutting Machine (a cutting machine manufactured by Dumbbell Co., Ltd (model SDL-200)), and a test piece having a dumbbel shape was prepared by cutting the polyimide (film) obtained in each Example or the like into a film having a size of the overall length: 75 mm, the distance between the tab portions: 57 mm, the length of the parallel portions: 30 mm, the radius of the shoulder portion: 30 mm, the width of the end portions: 10 mm, the width of the central parallel portion: 5 mm, and the thickness: 10 μm (which follows the standard of JIS K7139 Type A22 (reduced test piece) except that the thickness was 10 μm), and the test piece was used as a measurement sample. Subsequently, an electromechanical universal testing machine (model number “5943” manufactured by INSTRON) was used and the measurement sample was placed such that the width between the grips was 57 mm and the width of the gripped portion was 10 mm (the total width of the end portions), and then a tensile test of pulling the measurement sample under conditions of load cell: 1.0 kN and test speed: 5 mm/minute was conducted to obtain the values of the tensile elastic modulus and the fracture elongation. Note that as the test, the test according to JIS K7162 (published in 1994) was conducted. In addition, the value (% of the fracture elongation was obtained by calculating the following formula:

[fracture elongation (%)]={(L−L0)/L0}×100, where L0 represents the distance between the tab portions of the sample (=the width between grips: 57 mm) and L represents the distance between the tab portions of the sample until break (the width between the grips when the sample was broken: 57 mm+a).

### Example 4

First, a 30 ml three-necked flask was heated with a heat gun to be sufficiently dried. Next, the atmosphere gas in the three-necked flask which had been sufficiently dried was replaced with nitrogen to make the inside of the three-necked flask a nitrogen atmosphere. Subsequently, 0.6818 g of 4,4′-diaminobenzanilide (3.00 mmol: manufactured by Nipponjunryo Chemicals Co., Ltd.: hereinafter referred to simply as “DABAN”) was added as an aromatic diamine into the three-necked flask, and then 7.3397 g of N,N-dimethylacetamide was further added, followed by stirring to obtain a mixture liquid (part of DABAN was dissolved).

Next, 1.1531 g of the CpODA(CpODA-(A)) (3.00 mmol) obtained in Example 1 was added into the three-necked flask containing the solution under the nitrogen atmosphere, followed by stirring for 24 hours at room temperature (25° C.: the polymerization temperature of a polyamic acid) under the nitrogen atmosphere to obtain a reaction liquid. In this way, the polyamic acid was formed in the reaction liquid by using the CpODA-(A). Note that a dimethylacetamide solution in which the concentration of the polyamic acid was 0.5 g/dL was prepared by using part of the reaction liquid (the N,N-dimethylacetamide solution of the polyamic acid). Then, the intrinsic viscosity [η] of the polyamic acid, which was a reaction intermediate, was measured, and the intrinsic viscosity [η] of the polyamic acid was 0.913.

Subsequently, the reaction liquid was used as a coating liquid as it is, and the coating liquid (the reaction liquid) was spin-coated onto a glass plate (length: 100 mm, width: 100 mm) such that the thickness of a coating after the heat curing was 10 μm to form the coating on the glass plate. Subsequently, the glass plate with the coating formed was left to stand in an inert oven, and was heated for 0.5 hours under a temperature condition of 80° C. while nitrogen was caused to flow therein at 5 L/minute, and was then heated for 1 hour at a calcination temperature of 380° C. to cure the coating and form a film made of the polyimide on the glass plate. Subsequently, the glass plate on which the film made of the polyimide was formed was taken out of the inert oven and was immersed in 90° C. water for 1 hour, and the film made of the polyimide was peeled off the glass plate to obtain a colorless and transparent film (length 100 mm, width 100 mm, thickness 10 μm).

The IR spectrum of the film thus obtained was measured. FIG. 17 shows the IR spectrum of the compound forming the film obtained. As is clear from the result shown in FIG. 17 as well, the C═O stretching vibration of imidocarbonyl was observed at 1699 cm-1 in the compound forming the film obtained, indicating that the compound obtained was a polyimide. Table 3 presents the evaluation results of the characteristics of the polyimide (film) thus obtained and the characteristics of the polyamic acid of the intermediate.

Note that for the polyimide (film) obtained in Example 4, 20 samples each having a size of 3.0 mm in length and 3.0 mm in width were prepared by using the film and were placed in an aluminum-made sample pan, and the TG/DTA7200 thermogravimetric analyzer (manufactured by SII NanoTechnology Inc.) was used as a measurement device, the sample was heated under a condition of 10° c./min. in a range from room temperature (25° C.) to 600° C. while a nitrogen gas was caused to flow therethrough, and the temperature at which the weight of the sample was reduced by 5% was measured. The 5% weight loss temperature thus obtained of the polyimide (film) obtained in Example 4 was 501° C.

In addition, it is obvious from the content ratios of the isomers and the type of the aromatic diamine of the CpODA-(A) used in producing the polyimide that the polyimide obtained contained the repeating unit (II) represented by the general formula (7) and that the content of the repeating unit (II-A) having the trans-exo-exo three-dimensional structure and being represented by the general formula (8) was 84.2% by mol, and the content of the repeating unit (II-A) having the cis-exo-exo three-dimensional structure and being represented by the general formula (9) was 15.8% by mol, relative to the summed amount of the repeating unit (II).

### Example 5

A film made of a polyimide was obtained in the same manner as that in Example 4 except that a coating liquid obtained by adding and mixing triphenylphosphine (PPh₃) as an additive with the reaction liquid such that the ratio was 10 parts by mass relative to 100 parts by mass of the solid component of the polyamic acid in the reaction liquid was used instead of using the reaction liquid as it was and that the calcination temperature was changed to 350° C. Note that when the IR spectrum of the compound forming the film obtained was measured, the C═O stretching vibration of imidocarbonyl was observed, indicating that the compound obtained was a polyimide. Table 3 presents the evaluation result of the characteristics of the polyimide (film) thus obtained and the characteristics of the polyamic acid of the intermediate.

### Comparative Example 2

A film made of a polyimide was obtained in the same manner as that in Example 5 except that the CpODA (CpODA-(D)) obtained in Comparative Example 1 was used instead of the CpODA-(A). Note that when the IR spectrum of the compound forming the film obtained was measured, the C═O stretching vibration of imidocarbonyl was observed, indicating that the compound obtained was a polyimide. Table 3 presents the evaluation result of the characteristics of the polyimide (film) thus obtained and the characteristics of the polyamic acid of the intermediate.

Note that it is obvious from the content ratios of the isomers and the type of the aromatic diamine of the CpODA-(D) used in producing the polyimide that the polyimide obtained contained the repeating unit (II) represented by the general formula (7) but that the content of the repeating unit (II-A) having the trans-exo-exo three-dimensional structure and being represented by the general formula (8) was 0.4% by mol, and the content of the repeating unit (II-A) having the cis-exo-exo three-dimensional structure and being represented by the general formula (9) was 0.4% by mol, relative to the summed amount of the repeating unit (II).

TABLE 3

| | | | Polymerization | Intrinsic | | | | |
|---|---|---|---|---|---|---|---|---|
| | | | Temperature | Viscosity | | | | |
| | | | During | of | | | Linear | |
| | Type of | Type of | Production of | Polyamic | Additive | Calcination | Expansion | |
| | Tetracarboxylic | Aromatic | Polyamic Acid | Acid | (Parts by | Temperature | Coefficient | Tg |
| | Dianhydride | Diamine | (° C.) | (dL/g) | Mass) | (° C.) | (ppm/K) | (° C.) |
| Example 4 | CpODA-(A) | DABAN | 25 | 0.9 | Not Used | 380 | 11 | 357 |
| | (Example 1) | | | | | | | |
| Example 5 | CpODA-(A) | DABAN | 25 | 0.9 | PPh₃ | 350 | 15 | 364 |
| | (Example 1) | | | | (10) | | | |
| Comparative | CpODA-(D) | DABAN | 25 | 0.8 | PPh₃ | 350 | 20 | 350 |
| Example 2 | (Comparative | | | | (10) | | | |
| | Example 1) | | | | | | | |

| | Total | | | Dielectric | | | | |
|---|---|---|---|---|---|---|---|---|
| | Luminous | | Relative | Dissipation | | | Elastic | |
| | Transmittance | | Permittivity | Factor | Transmittance | | Modulus | Fracture |
| | (%) | YI | at 10 GHz | at 10 GHz | at 400 nm | Td5% | GPa | Elongation |
| Example 4 | 88.1 | 2.4 | 2.3 | 0.018 | 81 | 501 | 5.9 | 7 |
| Example 5 | 87.3 | 3.5 | 2.9 | 0.020 | 77 | 478 | 6.1 | 7 |
| Comparative | 87.8 | 2.8 | 2.4 | 0.026 | 82 | 474 | 5.1 | 13 |
| Example 2 | | | | | | | | |

As is clear from the result presented in Table 3, it was confirmed that Tg of all the polyimides obtained in Examples 4 and 5 was 250° C. or more, indicating that the polyimides had a sufficiently high level of heat resistance. In addition, the polyimides obtained in Examples 4 and 5 had total luminous transmittance of 80% or more, indicating that the polyimides had a sufficiently high level of transparency. Moreover, the polyimides obtained in Examples 4 and 5 had lower dielectric dissipation factor and lower CTE than those of the polyimide obtained in Comparative Example 2. Note that the polyimides obtained in Examples 4 and 5 had relative permittivity of 3.0 or less, indicating that the polyimides had sufficiently low relative permittivity.

### Example 6

A film made of a polyimide was obtained in the same manner as that in Example 5 except that the CpODA (CpODA-(B)) obtained in Example 2 was used instead of the CpODA-(A) and that a mixture of 0.5113 g of DABAN (2.25 mmol) and 0.0811 g of p-diaminobenzene (0.75 mmol, Paramine manufactured by Daishin chemical IND. co., LTD: hereinafter referred to simply as “PPD”) was used instead of solely using DABAN as an aromatic diamine. Note that when the IR spectrum of the compound forming the film obtained was measured, the C═O stretching vibration of imidocarbonyl was observed, indicating that the compound obtained was a polyimide. Table 4 presents the evaluation result of the characteristics of the polyimide (film) thus obtained and the characteristics of the polyamic acid of the intermediate.

Note that it is obvious from the content ratios of the isomers and the type of the aromatic diamine of the CpODA-(B) used in producing the polyimide that the polyimide obtained contained the repeating unit (II) represented by the general formula (7) and that the content of the repeating unit (II-A) having the trans-exo-exo three-dimensional structure and being represented by the general formula (8) was 95.8% by mol, and the content of the repeating unit (II-A) having the cis-exo-exo three-dimensional structure and being represented by the general formula (9) was 4.2% by mol, relative to the summed amount of the repeating unit (II).

### Comparative Example 3

A film made of a polyimide was obtained in the same manner as that in Example 6 except that the CpODA-(D) was used instead of the CpODA-(B). Note that when the IR spectrum of the compound forming the film obtained was measured, the C═O stretching vibration of imidocarbonyl was observed, indicating that the compound obtained was a polyimide. Table 4 presents the evaluation result of the characteristics of the polyimide (film) thus obtained and the characteristics of the polyamic acid of the intermediate.

TABLE 4

| | | Type of | | | | | | |
|---|---|---|---|---|---|---|---|---|
| | | Aromatic | Polymerization | Intrinsic | | | | |
| | | Diamine | Temperature | Viscosity | | | | |
| | | (Number in | During | of | | | Linear | |
| | Type of | Brackets is | Production of | Polyamic | Additive | Calcination | Expansion | |
| | Tetracarboxylic | Molar Ratio in | Polyamic Acid | Acid | (Parts by | Temperature | Coefficient | Tg |
| | Dianhydride | Mixture) | (° C.) | (dL/g) | Mass) | (° C.) | (ppm/K) | (° C.) |
| Example 6 | CpODA-(B) | Mixture of | 25 | 0.8 | PPh₃ | 350 | 18 | 351 |
| | (Example 2) | DABAN (75) | | | (10) | | | |
| | | and PPD (25) | | | | | | |
| Comparative | CpODA-(D) | Mixture of | 25 | 0.8 | PPh₃ | 350 | 19 | 336 |
| Example 3 | (Comparative | DABAN (75) | | | (10) | | | |
| | Example 1) | and PPD (25) | | | | | | |

| | Total | | | Dielectric | | | | |
|---|---|---|---|---|---|---|---|---|
| | Luminous | | Relative | Dissipation | | | Elastic | |
| | Transmittance | | Permittivity | Factor | Transmittance | | Modulus | Fracture |
| | (%) | YI | at 10 GHz | at 10 GHz | at 400 nm | Td5% | GPa | Elongation |
| Example 6 | 88.7 | 1.6 | 2.9 | 0.020 | 81 | 473 | 6.1 | 9 |
| Comparative | 88.2 | 1.9 | 2.6 | 0.025 | 81 | 473 | 5.4 | 14 |
| Example 3 | | | | | | | | |

As is clear from the result presented in Table 4, it was confirmed that Tg of the polyimide obtained in Example 6 was 250° C. or more, indicating that the polyimide had a sufficiently high level of heat resistance. In addition, the polyimide obtained in Example 6 had a total luminous transmittance of 80% or more, indicating that the polyimide had a sufficiently high level of transparency. Moreover, the polyimide obtained in Example 6 had lower dielectric dissipation factor and lower CTE than those of the polyimide obtained in Comparative Example 3. Note that the polyimide obtained in Example 6 had relative permittivity of 3.0 or less, indicating that the polyimide had sufficiently low relative permittivity.

### Example 7

A film made of a polyimide was obtained in the same manner as that in Example 5 except that the CpODA (CpODA-(C)) obtained in Example 3 was used instead of the CpODA-(A) and that a mixture of 0.3409 g of DABAN (1.50 mmol) and 0.1622 g of PPD (1.50 mmol) was used as an aromatic diamine instead of solely using DABAN as an aromatic diamine. Note that when the IR spectrum of the compound forming the film obtained was measured, the C═O stretching vibration of imidocarbonyl was observed, indicating that the compound obtained was a polyimide. Table 5 presents the evaluation result of the characteristics of the polyimide (film) thus obtained and the characteristics of the polyamic acid of the intermediate.

Note that it is obvious from the content ratios of the isomers and the type of the aromatic diamine of the CpODA-(C) used in producing the polyimide that the polyimide obtained contained the repeating unit (II) represented by the general formula (7) and that the content of the repeating unit (II-A) having the trans-exo-exo three-dimensional structure and being represented by the general formula (8) was 48.8% by mol, and the content of the repeating unit (II-A) having the cis-exo-exo three-dimensional structure and being represented by the general formula (9) was 51.2% by mol, relative to the summed amount of the repeating unit (II).

### Comparative Example 4

A film made of a polyimide was obtained in the same manner as that in Example 7 except that the CpODA-(D) was used instead of the CpODA-(C). Note that when the IR spectrum of the compound forming the film obtained was measured, the C═O stretching vibration of imidocarbonyl was observed, indicating that the compound obtained was a polyimide. Table 5 presents the evaluation result of the characteristics of the polyimide (film) thus obtained and the characteristics of the polyamic acid of the intermediate.

TABLE 5

| | | Type of | | | | | | |
|---|---|---|---|---|---|---|---|---|
| | | Aromatic | Polymerization | Intrinsic | | | | |
| | | Diamine | Temperature | Viscosity | | | | |
| | | (Number in | During | of | | | Linear | |
| | Type of | Brackets is | Production of | Polyamic | Additive | Calcination | Expansion | |
| | Tetracarboxylic | Molar Ratio in | Polyamic Acid | Acid | (Parts by | Temperature | Coefficient | Tg |
| | Dianhydride | Mixture) | (° C.) | (dL/g) | Mass) | (° C.) | (ppm/K) | (° C.) |
| Example 7 | CpODA-(C) | Mixture of | 25 | 0.8 | PPh₃ | 350 | 18 | 353 |
| | (Example 3) | DABAN (50) | | | (10) | | | |
| | | and PPD (50) | | | | | | |
| Comparative | CpODA-(D) | Mixture of | 25 | 0.9 | PPh₃ | 350 | 20 | 358 |
| Example 4 | (Comparative | DABAN (50) | | | (10) | | | |
| | Example 1) | and PPD (50) | | | | | | |

| | Total | | | Dielectric | | | | |
|---|---|---|---|---|---|---|---|---|
| | Luminous | | Relative | Dissipation | | | Elastic | |
| | Transmittance | | Permittivity | Factor | Transmittance | | Modulus | Fracture |
| | (%) | Yl | at 10 GHz | at 10 GHz | at 400 nm | Td5% | GPa | Elongation |
| Example 7 | 88.8 | 2.4 | 2.2 | 0.016 | 84 | 480 | 5.7 | 9 |
| Comparative | 85.1 | 4.9 | 2.2 | 0.031 | 72 | 479 | 3.9 | 17 |
| Example 4 | | | | | | | | |

As is clear from the result presented in Table 5, it was confirmed that Tg of the polyimide obtained in Example 7 was 250° C. or more, indicating that the polyimide had a sufficiently high level of heat resistance. In addition, the polyimide obtained in Example 7 had a total luminous transmittance of 80% or more, indicating that the polyimide had a sufficiently high level of transparency. Moreover, the polyimide obtained in Example 7 had lower dielectric dissipation factor and lower CTE than those of the polyimide obtained in Comparative Example 4. Note that the polyimide obtained in Example 7 had relative permittivity of 3.0 or less, indicating that the polyimide had sufficiently low relative permittivity.

### Example 8

A film made of a polyimide was obtained in the same manner as that in Example 5 except that a mixture of 0.3409 g of DABAN (1.50 mmol) and 0.4804 g of 2,2′-bis(trifluoromethyl)benzidine (1.50 mmol, manufactured by Seika Corporation: hereinafter referred to simply as “TFMB”) was used instead of solely using DABAN as an aromatic diamine. Note that when the IR spectrum of the compound forming the film obtained was measured, the C═O stretching vibration of imidocarbonyl was observed, indicating that the compound obtained was a polyimide. Table 6 presents the evaluation result of the characteristics of the polyimide (film) thus obtained and the characteristics of the polyamic acid of the intermediate.

### Comparative Example 5

A film made of a polyimide was obtained in the same manner as that in Example 8 except that the CpODA-(D) was used instead of the CpODA-(A). Note that when the IR spectrum of the compound forming the film obtained was measured, the C═O stretching vibration of imidocarbonyl was observed, indicating that the compound obtained was a polyimide. Table 6 presents the evaluation result of the characteristics of the polyimide (film) thus obtained and the characteristics of the polyamic acid of the intermediate.

TABLE 6

| | | Type of | | | | | | |
|---|---|---|---|---|---|---|---|---|
| | | Aromatic | Polymerization | Intrinsic | | | | |
| | | Diamine | Temperature | Viscosity | | | | |
| | | (Number in | During | of | | | Linear | |
| | Type of | Brackets is | Production of | Polyamic | Additive | Calcination | Expansion | |
| | Tetracarboxylic | Molar Ratio in | Polyamic Acid | Acid | (Parts by | Temperature | Coefficient | Tg |
| | Dianhydride | Mixture) | (° C.) | (dL/g) | Mass) | (° C.) | (ppm/K) | (° C.) |
| Example 8 | CpODA-(C) | Mixture of | 25 | 0.7 | PPh₃ | 350 | 18 | 350 |
| | (Example 3) | DABAN (50) | | | (10) | | | |
| | | and TFMB (50) | | | | | | |
| Comparative | CpODA-(D) | Mixture of | 25 | 0.4 | PPh₃ | 350 | 53 | 372 |
| Example 5 | (Comparative | DABAN (50) | | | (10) | | | |
| | Example 1) | and TFMB (50) | | | | | | |

| | Total | | | Dielectric | | | | |
|---|---|---|---|---|---|---|---|---|
| | Luminous | | Relative | Dissipation | | | Elastic | |
| | Transmittance | | Permittivity | Factor | Transmittance | | Modulus | Fracture |
| | (%) | YI | at 10 GHz | at 10 GHz | at 400 nm | Td5% | GPa | Elongation |
| Example 8 | 88.8 | 2.2 | 2.3 | 0.015 | 84 | 425 | 4.3 | 21 |
| Comparative | 89.4 | 1.4 | 2.1 | 0.023 | 86 | 424 | 3.3 | 34 |
| Example 5 | | | | | | | | |

As is clear from the result presented in Table 6, it was confirmed that Tg of the polyimide obtained in Example 8 was 250° C. or more, indicating that the polyimide had a sufficiently high level of heat resistance. In addition, the polyimide obtained in Example 6 had a total luminous transmittance of 80% or more, indicating that the polyimide had a sufficiently high level of transparency. Moreover, the polyimide obtained in Example 8 had lower dielectric dissipation factor and lower CTE than those of the polyimide obtained in Comparative Example 5. Note that the polyimide obtained in Example 8 had relative permittivity of 3.0 or less, indicating that the polyimide had sufficiently low relative permittivity.

### Examples 9 to 22

Films made of polyimides were obtained in the same manner as that in Example 4 except that aromatic diamines described in Table 7 were used respectively in molar amounts described in Table 7 instead of solely using DABAN as an aromatic diamine, that temperatures described in Table 7 were employed as polymerization temperatures during polymerization of a polyamic acid, and that the calcination temperature was changed to the calcination temperatures described in Table 7, respectively. Note that from the measurement result of the IR spectrum of the compound forming the film obtained in each Example, the C═O stretching vibration of imidocarbonyl was observed for each film, indicating that the compound obtained was a polyimide. Table 7 presents the evaluation result of the characteristics of each polyimide (film) thus obtained and the characteristics of the polyamic acid of the intermediate.

<Regarding Abbreviations of Aromatic Diamines in Table 7>

Regarding the aromatic diamines described in Table 7, abbreviated names of aromatic diamines such as DABAN, PPD, and TFMB are as described above, and as abbreviated names of the other components, those described in the following (1) to (12) (showing abbreviated names and compound names together) were used:

(1) 4-APBP: 4,4′-bis(4-aminophenoxy)biphenyl (manufactured by Nipponjunryo Chemicals Co., Ltd.), (2) HFBAPP: 2,2-bis[4-(4-aminophenoxy)phenyl]hexafluoropropane (manufactured by Seika Corporation), (3) TPE-R: 1,3-bis(4-aminophenoxy)benzene (manufactured by Seika Corporation), (4) BAPP: 2,2′-bis[4-(4-aminophenoxy)phenyl]propane (manufactured by Seika Corporation), (5) Terphenyl: 4,4″-diamino-p-terphenyl (manufactured by Tokyo Chemical Industry Co., Ltd.), (6) TPE-Q: 1,4-bis(4-aminophenoxy)benzene (manufactured by Seika Corporation), (7) APB-N: 1,3-bis(3-aminophenoxy)benzene (manufactured by MITSUI FINE CHEMICALS, INC.), (8) m-tol: 4,4′-diamino-2,2′-dimethylbiphenyl (manufactured by Seika Corporation), (9) BAPS: bis[4-(4-aminophenoxy)phenyl]sulfone (manufactured by Seika Corporation), (10) BAPS-M: bis[4-(3-aminophenoxy)phenyl]sulfone (manufactured by Seika Corporation), (11) BAPK: 4,4′-bis(4-aminophenoxy)benzophenone (manufactured by Seika Corporation), (12) DDE: 4,4′-diaminodiphenyl ether (manufactured by Tokyo Chemical Industry Co., Ltd.).

TABLE 7

| | | Type of | | | | |
|---|---|---|---|---|---|---|
| | | Aromatic | | Polymerization | Intrinsic | |
| | | Diamine | Molar | Temperature | Viscosity | |
| | | (Number in | Amount of | During | of | |
| | Type of | Brackets is | Aromatic | Production of | Polyamic | Calcination |
| | Tetracarboxylic | Molar Ratio in | Diamine | Polyamic Acid | Acid | Temperature |
| | Dianhydride | Mixture) | (mmol) | (° C.) | (dL/g) | (° C.) |
| Example 9 | CpODA-(A) | Mixture of | 3.00 | 25 | 0.7 | 350 |
| | (Example 1) | DABANC75) | | | | |
| | | and | | | | |
| | | 4-APBP(25) | | | | |
| Example 10 | CpODA-(A) | TFMB | 3.00 | 60 | 0.4 | 350 |
| | (Example 1) | | | | | |
| Example 11 | CpODA-(A) | 4-APBP | 3.00 | 25 | 0.9 | 350 |
| | (Example 1) | | | | | |
| Example 12 | CpODA-(A) | HFBAPP | 3.00 | 25 | 0.7 | 300 |
| | (Example 1) | | | | | |
| Example 13 | CpODA-(A) | TPE-R | 3.00 | 25 | 0.8 | 360 |
| | (Example 1) | | | | | |
| Example 14 | CpODA-(A) | BAPP | 3.00 | 25 | 0.5 | 350 |
| | (Example 1) | | | | | |
| Example 15 | CpODA-(A) | Terphenyl | 3.00 | 25 | 1.1 | 400 |
| | (Example 1) | | | | | |
| Example 16 | CpODA-(A) | TPE-Q | 3.00 | 25 | 1.0 | 360 |
| | (Example 1) | | | | | |
| Example 17 | CpODA-(A) | APB-N | 3.00 | 25 | 0.3 | 350 |
| | (Example 1) | | | | | |
| Example 18 | CpODA-(A) | m-Tol | 3.00 | 25 | 1.5 | 350 |
| | (Example 1) | | | | | |
| Example 19 | CpODA-(A) | BAPS | 3.00 | 25 | 0.6 | 350 |
| | (Example 1) | | | | | |
| Example 20 | CpODA-(A) | BAPS-M | 3.00 | 25 | 0.2 | 350 |
| | (Example 1) | | | | | |
| Example 21 | CpODA-(A) | BAPK | 3.00 | 25 | 0.9 | 350 |
| | (Example 1) | | | | | |
| Example 22 | CpODA-(A) | DDE | 3.00 | 25 | 1.0 | 360 |
| | (Example 1) | | | | | |

| | Linear | | | | | Dielectric |
|---|---|---|---|---|---|---|
| | Expansion | | Total Luminous | | Relative | Dissipation |
| | Coefficient | Tg | Transmittance | | Permittivity | Factor |
| | (ppm/K) | (° C.) | (%) | YI | at 10 GHz | at 10 GHz |
| Example 9 | 17 | 347 | 88.3 | 1.8 | 2.6 | 0.021 |
| Example 10 | 53 | 351 | 90.3 | 1.3 | 2.5 | 0.010 |
| Example 11 | 55 | 295 | 88.7 | 2.5 | 2.4 | 0.008 |
| Example 12 | 58 | 336 | 89.1 | 1.5 | 2.1 | 0.019 |
| Example 13 | 60 | 288 | 89.7 | 1.4 | 2.4 | 0.007 |
| Example 14 | 68 | 294 | 89.4 | 1.5 | 2.1 | 0.015 |
| Example 15 | 8 | 398 | 86.1 | 4.1 | 2.6 | 0.010 |
| Example 16 | 56 | 328 | 88.0 | 2.9 | 2.9 | 0.008 |
| Example 17 | 65 | 265 | 89.7 | 0.8 | 2.4 | 0.008 |
| Example 18 | 33 | 410 | 89.5 | 1.5 | 2.7 | 0.009 |
| Example 19 | 65 | 364 | 89.2 | 1.2 | 2.5 | 0.020 |
| Example 20 | 61 | 327 | 89.6 | 0.9 | 2.4 | 0.012 |
| Example 21 | 59 | 298 | 87.8 | 4.8 | 2.7 | 0.011 |
| Example 22 | 46 | 369 | 89.2 | 0.7 | 2.4 | 0.015 |

As is clear from the results presented in Table 7 as well, it was confirmed that Tg of all the polyimides of the present invention (Examples 9 to 22) was 250° C. or more, indicating that the polyimides had a sufficiently high level of heat resistance. In addition, all the polyimides of the present invention (Examples 9 to 22) had a total luminous transmittance of 80% or more, indicating that the polyimides had a sufficiently high level of transparency. Moreover, all the polyimides of the present invention (Examples 9 to 22) had CTE of 70 ppm/K or less, which was sufficiently low CTE. In addition, all the polyimides of the present invention (Examples 9 to 22) had dielectric dissipation factor of values lower than 0.023 (having values of 0.022 or less), which was sufficiently low dielectric dissipation factor. Note that all the polyimides of the present invention (Examples 9 to 22) had relative permittivity of 3.0 or less, indicating that the polyimides had sufficiently low relative permittivity. Note that when the refractive index of the polyimide obtained in Example 15 to light having a wavelength of 594 nm was obtained in TE mode using “Prism Coupler 2010/M” manufactured by Metricon Corporation as a measurement device, the refractive index was 1.6957. In addition, the refractive index of the polyimide obtained in Example 15 to light having a wavelength of 540 nm was obtained by employing the same method except that the wavelength was changed to 540 nm, the refractive index was 1.7200.

As described above, in the results presented in Tables 3 to 6, when those using the same aromatic diamine were compared, it was confirmed that all the polyimides of the present invention (Examples 4 to 7) that comprised the repeating unit (II) wherein the content of the repeating unit (II-A) having the trans-exo-exo three-dimensional structure was 40% by mol or more and the content of the repeating unit (II-B) having the cis-exo-exo isomer three-dimensional structure was 2% by mol or more had lower CTE and dielectric dissipation factor than those of the polyimides (Comparative Examples 2 to 5) wherein the content of the repeating unit (II-A) having the trans-exo-exo three-dimensional structure was less than 40% by mol and the content of the repeating unit (II-B) having the cis-exo-exo isomer three-dimensional structure was less than 2% by mol. From these results, it was found that it was possible to make CTE and dielectric dissipation factor lower depending on the contents of the repeating unit (II-A) and the repeating unit (II-B). In addition, as is clear from the results presented in Tables 3 to 7 as well, all the polyimides of the present invention (Examples 4 to 22) had Tg of 250° C. or more (the polyimide of each Example actually had Tg of 265° C. or more), and it was also confirmed that these polyimides had a sufficiently high level of heat resistance based on Tg. Moreover, as is clear from the results presented in Tables 3 to 7 as well, all the polyimides of the present invention (Examples 4 to 22) had total luminous transmittance of 80% or more, it was confirmed that the polyimides had a sufficiently high level of transparency. Note that as is clear from the results presented in Tables 3 to 7 as well, all the polyimides of the present invention (Examples 4 to 22) had dielectric dissipation factor of a low value of 0.022 or less and relative permittivity of a sufficiently low value of 3.0 or less, it is understood that it is possible to apply the films made of the polyimides to a high-frequency material and the like.

### Example 23

A film made of a polyimide was obtained in the same manner as that in Example 4 except that a mixture of 0.5454 g of DABAN (2.40 mmol) and 0.1274 g of m-Tol (0.60 mmol) was used instead of solely using DABAN as an aromatic diamine, that a coating liquid obtained by adding and mixing 1,2-dimethylimidazole as an additive with the reaction liquid such that the ratio was 10 parts by mass relative to 100 parts by mass of the solid component of the polyamic acid in the obtained reaction liquid was used instead of using the reaction liquid as it was as a coating liquid, and that the calcination temperature was changed to 350° C. Note that in the measurement result of the IR spectrum of the compound forming the film thus obtained, the C═O stretching vibration of imidocarbonyl was observed, indicating that the compound obtained was a polyimide. Table 8 presents the evaluation result of the characteristics of the polyimide (film) thus obtained and the characteristics of the polyamic acid of the intermediate. Note that abbreviated names of aromatic diamines have the same definitions as those described in Table 7.

### Examples 24 to 27

Films made of polyimides were obtained in the same manner as that in Example 23 except that aromatic diamines described in Table 8 were used respectively in molar amounts described in Table 8 instead of using a mixture of DABAN and m-Tol as an aromatic diamine and that the calcination temperatures described in Table 8 were employed respectively as calcination temperatures. Note that from the measurement result of the IR spectrum of the compound forming the film obtained in each Example, the C═O stretching vibration of imidocarbonyl was observed for each film, indicating that the compound obtained was a polyimide. Table 8 presents the evaluation result of the characteristics of each polyimide (film) thus obtained and the characteristics of the polyamic acid of the intermediate. Note that regarding abbreviated names of the aromatic diamines in Table 8, abbreviated names of aromatic diamines such as DABAN, PPD, and TFMB are as described above, “ASD” is an abbreviated name of bis(4-aminophenyl) sulfide, “BAAB” is an abbreviated name of 4-aminophenyl-4-aminobenzoic acid, and abbreviated names of the other aromatic diamines have the same definitions as those described in Table 7.

### Example 28

A film made of a polyimide was obtained in the same manner as that in Example 4 except that 2-phenyl-4-aminophenyl)-4-aminobenzoate (hereinafter referred to simply as “4-PHBAAB”) was used in molar amount described in Table 8 instead of solely using DABAN as an aromatic diamine, that the polymerization temperature during polymerization (production) of a polyamic acid was changed to 60° C., and that the calcination temperature was changed to 350° C. Note that in the measurement result of the IR spectrum of the compound forming the film thus obtained, the C═O stretching vibration of imidocarbonyl was observed, indicating that the compound obtained was a polyimide. Table 8 presents the evaluation result of the characteristics of the polyimide (film) thus obtained and the characteristics of the polyamic acid of the intermediate.

### Example 29

A film made of a polyimide was obtained in the same manner as that in Example 4 except that the CpODA-(B) was used as a tetracarboxylic dianhydride instead of using the CpODA-(A), that 1,3-bis[2-(4-aminophenyl)-2-propyl]benzene (alias “bisaniline M”) was used in molar amount described in Table 8 instead of using DABAN as an aromatic diamine, and that the calcination temperature was changed to the calcination temperature described in Table 8. Note that in the measurement result of the IR spectrum of the compound forming the film thus obtained, the C═O stretching vibration of imidocarbonyl was observed, indicating that the compound obtained was a polyimide. Table 8 presents the evaluation result of the characteristics of the polyimide (film) thus obtained and the characteristics of the polyamic acid of the intermediate.

### Example 30

A film made of a polyimide was obtained in the same manner as that in Example 23 except that 1,4-bis[2-(4-aminophenyl)-2-propyl]benzene (alias “bisaniline P”) was used in molar amount described in Table 8 instead of using DABAN as an aromatic diamine and that the calcination temperature was changed to the calcination temperature described in Table 8. Note that in the measurement result of the IR spectrum of the compound forming the film thus obtained, the C═O stretching vibration of imidocarbonyl was observed, indicating that the compound obtained was a polyimide. Table 8 presents the evaluation result of the characteristics of the polyimide (film) thus obtained and the characteristics of the polyamic acid of the intermediate.

### Example 31

A film made of a polyimide was obtained in the same manner as that in Example 4 except that a mixture of the CPODA-(A), 4,4′-(hexafluoroisopropylidene)diphthalic anhydride (hereinafter referred to simply as “6FDA”), 3,3′,4,4′-biphenyltetracarboxylic dianhydride (hereinafter referred to simply as “BPDA”), and pyromellitic dianhydride (hereinafter referred to simply as “PMDA”) (total molar amount: 3.00 mmol) was used such that the molar ratios (CPODA-(A):6FDA:BPDA:PMDA) of the respective components in the mixture were 30:30:30:10 instead of solely using the CpODA-(A) as a tetracarboxylic dianhydride and that the aromatic diamine described in Table 8 was used in molar amount described in Table 8 instead of solely using DABAN as an aromatic diamine. Note that in the measurement result of the IR spectrum of the compound forming the film thus obtained, the C═O stretching vibration of imidocarbonyl was observed, indicating that the compound obtained was a polyimide. Table 8 presents the evaluation result of the characteristics of the polyimide (film) thus obtained and the characteristics of the polyamic acid of the intermediate. Note that regarding abbreviated names of the aromatic diamines in Table 8, “4,4′-DDS” represents 4,4′-diaminodiphenyl sulfone and abbreviated names of the other aromatic diamines have the same definitions as those described in Table 7.

### Example 32

A film made of a polyimide was obtained by employing the same method as that in Example 4 except that a reaction liquid (a polyimide solution) obtained by employing a “step of preparing a polyimide solution” described below instead of using the reaction liquid (the N,N-dimethylacetamide solution of the polyamic acid) as a coating liquid and that the calcination temperature was changed to 360° C. Note that in the measurement result of the IR spectrum of the compound forming the film thus obtained, the C═O stretching vibration of imidocarbonyl was observed, indicating that the compound obtained was a polyimide. Table 8 presents the evaluation result of the characteristics of the polyimide (film) thus obtained.

<Step of Preparing Polyimide Solution>

First, a 30 ml three-necked flask was heated with a heat gun to be sufficiently dried. Next, the atmosphere gas in the three-necked flask which had been sufficiently dried was replaced with nitrogen to make the inside of the three-necked flask a nitrogen atmosphere. Subsequently, 1.0453 g of 9,9-bis(4-aminophenyl)fluorene (3.00 mmol: manufactured by Seika Corporation: hereinafter referred to simply as “FDA”) was added as an aromatic diamine into the three-necked flask, and then a mixed solvent of 1.759 g of N,N-dimethylacetamide (DMAc) and 7.035 g of y-butyrolactone (γ-BL) (mass ratio: γ-BL/DMAc=4/1) as well as 15.2 mg of triethylamine (0.15 mmol, 5 mol %) were further added, followed by stirring to obtain a mixture liquid. Next, 1.1531 g of the CpODA (CpODA-(A)) (3.00 mmol) obtained in Example 1 was added into the three-necked flask containing the mixture liquid under the nitrogen atmosphere, followed by heating and stirring for 3 hours at 180° C. under the nitrogen atmosphere to obtain a reaction liquid. Note that it is obvious that in the mixture liquid, such heating first caused reaction between the aromatic diamine (FDA) and the tetracarboxylic dianhydride component (CpODA(A)) to proceed to form a polyamic acid, and Subsequently the imidization proceeded to form the polyimide. In this way, the reaction liquid made of the polyimide solution was obtained by conducting the heating step (polymerization temperature of the polyimide: 180° C.). Note that the intrinsic viscosity [η] of the polyimide was measured by employing the same method as the above-described measuring method of the intrinsic viscosity [η] except that after the polyimide was isolated by using part of the reaction liquid (polyimide solution), a DMAc solution in which the concentration of the polyimide was 0.5 g/dL was prepared as a measurement sample. The intrinsic viscosity [η] of the polyimide was 0.45.

TABLE 8

| | | Type of | | | | | |
|---|---|---|---|---|---|---|---|
| | | Aromatic | | Polymerization | Intrinsic | | |
| | | Diamine | Molar | Temperature | Viscosity | | |
| | | (Number in | Amount of | During | of | | |
| | Type of | Brackets is | Aromatic | Production of | Polyamic | | Calcination |
| | Tetracarboxylic | Molar Ratio in | Diamine | Polyamic Acid | Acid | | Temperature |
| | Dianhydride | Mixture) | (mmol) | (° C.) | (dL/g) | Additive | (° C.) |
| Example 23 | CpODA-(A) | Mixture of | 3.00 | 25 | 0.8 | 1,2- | 350 |
| | (Example 1) | DABAN (80) and | | | | Dimethylimidazole | |
| | | m-Tol (20) | | | | | |
| Example 24 | CpODA-(A) | Mixture of | 3.00 | 25 | 0.7 | 1,2- | 350 |
| | (Example 1) | TFMB (60) and | | | | Dimethylimidazole | |
| | | m-Tol (40) | | | | | |
| Example 25 | CpODA-(A) | Mixture of | 3.00 | 25 | 0.8 | 1,2- | 350 |
| | (Example 1) | PPD (80) and | | | | Dimethylimidazole | |
| | | TPE-R (20) | | | | | |
| Example 26 | CpODA-(A) | ASD | 3.00 | 25 | 0.4 | 1,2- | 300 |
| | (Example 1) | | | | | Dimethylimidazole | |
| Example 27 | CpODA-(A) | Mixture of | 3.00 | 25 | 0.7 | 1,2- | 350 |
| | (Example 1) | DABAN (80) and | | | | Dimethylimidazole | |
| | | BAAB (20) | | | | | |
| Example 28 | CpODA-(A) | 4-PHBAAB | 3.00 | 60 | 0.2 | Not Used | 350 |
| | (Example 1) | | | | | | |
| Example 29 | CpODA-(B) | Bisaniline M | 3.00 | 25 | 0.4 | Not Used | 340 |
| | (Example 2) | | | | | | |
| Example 30 | CpODA-(A) | Bisaniline P | 3.00 | 25 | 0.5 | 1,2- | 350 |
| | (Example 1) | | | | | Dimethylimidazole | |
| Example 31*¹ | CpODA-(A) (30) | Mixture of | 3.00 | 25 | 0.3 | Not Used | 380 |
| | 6FDA (30) | TFMB (75) and | | | | | |
| | BPDA (30) | 4,4′-DDS (25) | | | | | |
| | PMDA (10) | | | | | | |
| Example 32*² | CpODA-(A) | FDA | 3.00 | 180 | 0.45 | Triethylamine | 360 |
| | (Example 1) | | | (Polymerization | (Intrinsic | | |
| | | | | Temperature of | Viscosity of | | |
| | | | | Polyimide) | Polyimide) | | |

| | | | Total | | | | | | | |
|---|---|---|---|---|---|---|---|---|---|---|
| | Linear | | Luminous | | | Dielectric | | | | |
| | Expansion | | Transmit- | | Relative | Dissipation | | | Elastic | |
| | Coefficient | Tg | tance | | Permittivity | Factor | Transmittance | Td5% | Modulus | Fracture |
| | (ppm/K) | (° C.) | (%) | YI | at 10 GHz | at 10 GHz | at 400 nm | (° C.) | (GPa) | Elongation |
| Example 23 | 15 | 430 | 88.2 | 2.9 | 2.9 | 0.016 | 81.4 | 474 | 6.3 | 14.2 |
| Example 24 | 11 | 427 | 90.3 | 1.2 | 2.5 | 0.011 | 86.1 | 471 | 3.8 | 9.8 |
| Example 25 | 26 | 425 | 89.1 | 1.9 | 3.0 | 0.015 | 85.3 | 479 | 4.3 | 14.6 |
| Example 26 | 49 | 400 | 89.3 | 2.3 | 2.7 | 0.013 | 87.0 | 479 | 3.0 | 17.3 |
| Example 27 | 14 | 391 | 87.2 | 2.7 | 3.1 | 0.010 | 73.7 | 483 | 4.8 | 1.6 |
| Example 28 | 36 | 292 | 80.3 | 4.2 | 2.9 | 0.005 | 72.0 | 468 | 5.6 | 1.5 |
| Example 29 | 61.0 | 305 | 89.1 | 1.3 | 2.3 | 0.005 | 81.3 | 489 | 3.0 | 11.3 |
| Example 30 | 54 | 407 | 89.9 | 0.9 | 2.8 | 0.011 | 85.6 | 481 | 2.7 | 39.9 |
| Example 31*¹ | 20 | 368 | 89.6 | 3.1 | 3.0 | 0.011 | 76.5 | 481 | 2.0 | 11 |
| Example 32*² | 49 | >455 | 89.5 | 0.6 | 2.9 | 0.021 | 88.2 | 506 | 2.8 | 7.7 |

In Table,
*¹indicates that the numerical value in parentheses described together with the abbreviation of the tetracarboxylic dianhydride is the molar ratio. In addition, in Table,
*²indicates that since not a polyamic acid solution but a polyimide solution was used as the coating liquid (reaction liquid) in Example 32, the polymerization temperature of the polyimide during preparation of the reaction liquid is described in the column of Polymerization Temperature During Production of Polyamic Acid, and the intrinsic viscosity of the polyimide is described in the column of Intrinsic Viscosity of Polyamic Acid.

As is clear from the results presented in Table 8 as well, Tg of all the polyimides of the present invention (Examples 23 to 32) were 250° C. or more, indicating that the polyimides had a sufficiently high level of heat resistance. In addition, all the polyimides of the present invention (Examples 23 to 32) had total luminous transmittance of 80% or more, indicating that the polyimides had a sufficiently high level of transparency. Moreover, all the polyimides of the present invention (Examples 23 to 32) had CTE of 70 ppm/K or less, which was sufficiently low CTE. In addition, all the polyimides of the present invention (Examples 23 to 32) had dielectric dissipation factor of values lower than 0.023 (having values of 0.022 or less), which was sufficiently low dielectric dissipation factor. Note that when the refractive index of the polyimide obtained in Example 26 to light having a wavelength of 594 nm was obtained in TE mode using “Prism Coupler 2010/M” manufactured by Metricon Corporation as a measurement device, the refractive index was 1.6182.

### Examples 33 to 39

Films made of polyimides were obtained in the same manner as that in Example 23 except that aromatic diamines described in Table 9 were used respectively in molar amounts described in Table 9 instead of using a mixture of DABAN and m-Tol as an aromatic diamine and that the calcination temperature was changed to the calcination temperatures described in Table 9, respectively. Note that as a result of measuring the IR spectrum of the compound forming the film obtained in each Example, the C═O stretching vibration of imidocarbonyl was observed for each film, indicating that the compound obtained was a polyimide. Table 9 presents the evaluation result of the characteristics of each polyimide (film) thus obtained and the characteristics of the polyamic acid of the intermediate. Note that regarding abbreviated names of the aromatic diamines in Table 9, “3,3′-DDS” is an abbreviated name of 3,3′-diaminodiphenyl sulfone and abbreviated names of the other aromatic diamines have the same definition as those described in Table 7 and Table 8.

TABLE 9

| | | Type of | | | | | |
|---|---|---|---|---|---|---|---|
| | | Aromatic | | Polymerization | Intrinsic | | |
| | | Diamine | Molar | Temperature | Viscosity | | |
| | | (Number in | Amount of | During | of | | |
| | Type of | Brackets is | Aromatic | Production of | Polyamic | | Calcination |
| | Tetracarboxylic | Molar Ratio in | Diamine | Polyamic Acid | Acid | | Temperature |
| | Dianhydride | Mixture) | (mmol) | (° C.) | (dL/g) | Additive | (° C.) |
| Example 33 | CpODA-(A) | Mixture of | 3.00 | 25 | 0.9 | 1,2- | 350 |
| | (Example 1) | DABAN (80) and | | | | Dimethylimidazole | |
| | | TFMB (20) | | | | | |
| Example 34 | CpODA-(A) | Mixture of | 3.00 | 25 | 0.8 | 1,2- | 350 |
| | (Example 1) | TFMB (60) and | | | | Dimethylimidazole | |
| | | PPD(40) | | | | | |
| Example 35 | CpODA-(A) | Mixture of | 3.00 | 25 | 0.8 | 1,2- | 350 |
| | (Example 1) | PPD (80) and | | | | Dimethylimidazole | |
| | | DDE (20) | | | | | |
| Example 36 | CpODA-(A) | 3,3′-DDS | 3.00 | 25 | 0.2 | 1,2- | 300 |
| | (Example 1) | | | | | Dimethylimidazole | |
| Example 37 | CpODA-(A) | 4,4′-DDS | 3.00 | 25 | 0.2 | 1,2- | 300 |
| | (Example 1) | | | | | Dimethylimidazole | |
| Example 38 | CpODA-(A) | FDA | 3.00 | 25 | 0.4 | 1,2- | 300 |
| | (Example 1) | | | | | Dimethylimidazole | |
| Example 39 | CpODA-(A) | Mixtuire of | 3.00 | 25 | 0.6 | 1,2- | 400 |
| | (Example 1) | FDA (80) and | | | | Dimethylimidazole | |
| | | DABAN (20) | | | | | |

| | Linear | | Total | | | | | | |
|---|---|---|---|---|---|---|---|---|---|
| | Expansion | | Luminous | | Relative | | | Elastic | |
| | Coefficient | Tg | Transmittance | | Permittivity | Transmittance | Td5% | Modulus | Fracture |
| | (ppm/K) | (° C.) | (%) | YI | at 10 GHz | at 400 nm | (° C.) | (GPa) | Elongation |
| Example 33 | 12 | 428 | 88.4 | 2.4 | 2.8 | 81.4 | 478 | 5.2 | 11.1 |
| Example 34 | 13 | 434 | 90.3 | 1.3 | 2.5 | 87.3 | 492 | 4.2 | 6.5 |
| Example 35 | 17 | 444 | 89.1 | 1.6 | 3.0 | 85.0 | 500 | 4.0 | 14.3 |
| Example 36 | 42 | 314 | 87.0 | 3.9 | 3.1 | 75.0 | 450 | — | 4.2 |
| Example 37 | 46 | 440 | 89.6 | 1.4 | 3.0 | 86.0 | 442 | 0.8 | 7.9 |
| Example 38 | 46 | >455 | 89.4 | 0.6 | 3.0 | 85.0 | 503 | 1.3 | 13 |
| Example 39 | 33 | 455 | 89.9 | 2.4 | 3.1 | 82.0 | 496 | 2.9 | 11.2 |

As is clear from the results presented in Table 9 as well, Tg of all the polyimides of the present invention (Examples 33 to 39) were 250° C. or more, indicating that the polyimides had a sufficiently high level of heat resistance. In addition, all the polyimides of the present invention (Examples 33 to 39) had total luminous transmittance of 80% or more, indicating that the polyimides had a sufficiently high level of transparency. Moreover, all the polyimides of the present invention (Examples 23 to 39) had CTE of 70 ppm/K or less, which was sufficiently low CTE. Note that when the refractive indices of the polyimides obtained in Examples 37 and 38 to light having a wavelength of 594 nm was obtained in TE mode using “Prism Coupler 2010/M” manufactured by Metricon Corporation as a measurement device, the refractive indices was 1.5949 (Example 37) and 1.6062 (Example 38), respectively.

As is clear from the results presented in Tables 3 to 9 as well, it was confirmed that Tg of all the polyimides (Examples 23 to 39) presented in Tables 8 and 9 was 250° C. or more like the polyimides (Examples 4 to 22) presented in Tables 3 to 7, and it was also confirmed that these polyimides had a sufficiently high level of heat resistance based on Tg. Moreover, all the polyimides (Examples 23 to 39) presented in Tables 8 and 9 had total luminous transmittance of 80% or more like the polyimides (Examples 4 to 22) presented in Tables 3 to 7, it was confirmed that the polyimides had a sufficiently high level of transparency.

### INDUSTRIAL APPLICABILITY

As described above, the present invention makes it possible to provide a tetracarboxylic dianhydride that can be used as a raw material monomer for producing a polyimide that can have lower linear expansion coefficient and dielectric dissipation factor while having sufficiently high levels of heat resistance and transparency; a polyimide precursor resin that can be efficiently produced by using the tetracarboxylic dianhydride and can be used for producing a polyimide that can have lower linear expansion coefficient and dielectric dissipation factor while having sufficiently high levels of heat resistance and transparency; and a polyimide that can have lower linear expansion coefficient and dielectric dissipation factor while having sufficiently high levels of heat resistance and transparency. In addition, the present invention also makes it possible to provide a polyimide precursor resin solution comprising the polyimide precursor resin; a polyimide solution comprising the polyimide; and a polyimide film that is obtained by using these solutions.

Hence, the polyimide of the present invention is particularly useful as materials for producing high-frequency materials, low dielectric materials, Low-k multilayer interconnect materials, semiconductors, low dielectric resin materials for electronic devices, printed wiring board materials, interlayer insulating film materials, flexible printed wiring board materials, semiconductor resist materials, photosensitive resist materials, permanent insulating film materials, and the like.

1. A tetracarboxylic dianhydride that is a mixture of stereoisomers of a compound represented by the following general formula (1): 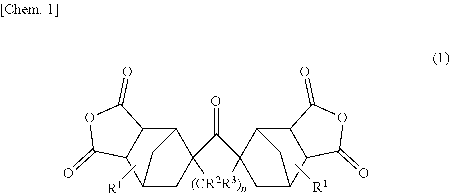
[in the formula (1), R¹, R², and R³ each independently represent one selected from the group consisting of a hydrogen atom, alkyl groups having 1 to 10 carbon atoms, and a fluorine atom, and n is an integer of 0 to 12], wherein
a content of an isomer (A) represented by the following general formula (2): 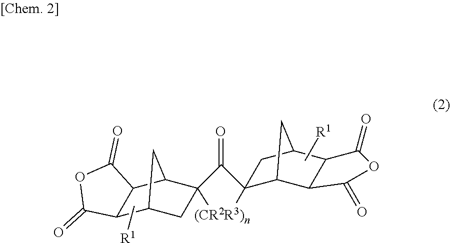
[R¹, R², R³, and n in the formula (2) have the same definitions as those of R¹, R², R³, and n in the general formula (1), respectively] is 40% by mol to 98% by mol relative to a total amount of the stereoisomers,
a content of an isomer (B) represented by the following general formula (3): 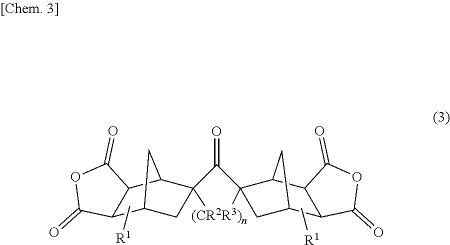
[R¹, R², R³, and n in the formula (3) have the same definitions as those of R¹, R², R³, and n in the general formula (1), respectively] is 2% by mol to 60% by mol relative to the total amount of the stereoisomers, and
a summed amount of the isomers (A) and (B) is 42% by mol or more relative to the total amount of the stereoisomers.

2. A polyimide precursor resin comprising a repeating unit (I) represented by the following general formula (4): 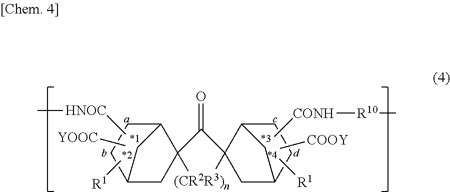
[in the formula (4), R¹, R², and R³ each independently represent one selected from the group consisting of a hydrogen atom, alkyl groups having 1 to 10 carbon atoms, and a fluorine atom, n represents an integer of 0 to 12, R¹⁰ represents an arylene group having 6 to 50 carbon atoms, Ys each independently represent one selected from the group consisting of a hydrogen atom, alkyl groups having 1 to 6 carbon atoms, and alkyl silyl groups having 3 to 9 carbon atoms, one of a bonding arm represented by *1 and a bonding arm represented by *2 is bonded to a carbon atom a forming the norbornane ring, the other of the bonding arm represented by *1 and the bonding arm represented by *2 is bonded to a carbon atom b forming the norbornane ring, one of a bonding arm represented by *3 and a bonding arm represented by *4 is bonded to a carbon atom c forming the norbornane ring, and the other of the bonding arm represented by *3 and the bonding arm represented by *4 is bonded to a carbon atom d forming the norbornane ring], wherein
a content of a repeating unit (I-A) having a three-dimensional structure represented by the following general formula (5): 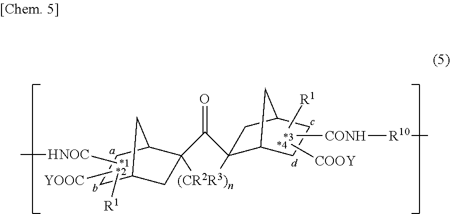
[R¹, R², R³, R¹⁰, Y, n, a to d, and *1 to *4 in the formula (5) have the same definitions as those of R¹, R², R³, R¹⁰, Y, n, a to d, and *1 to *4 in the general formula (4), respectively] is 40% by mol to 98% by mol relative to a total amount of the repeating unit (I),
a content of a repeating unit (I-B) having a three-dimensional structure represented by the following general formula (6): 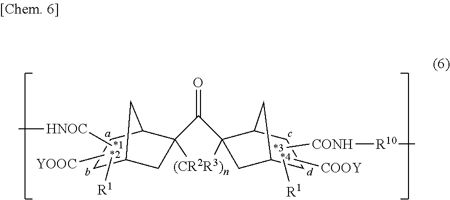
[R¹, R², R³, R¹⁰, Y, n, a to d, and *1 to *4 in the formula (6) have the same definitions as those of R¹, R², R³, R¹⁰, Y, n, a to d, and *1 to *4 in the general formula (4), respectively] is 2% by mol to 60% by mol relative to the total amount of the repeating unit (I), and
a summed amount of the repeating units (I-A) and (I-B) is 42% by mol or more relative to the total amount of the repeating unit (1).

3. A polyimide comprising a repeating unit (II) represented by the following general formula (7): 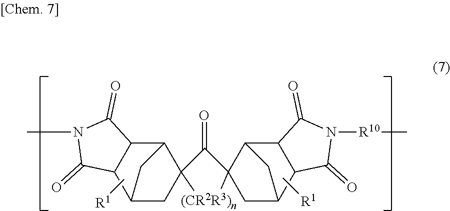
[in the formula (7), R¹, R², and R³ each independently represent one selected from the group consisting of a hydrogen atom, alkyl groups having 1 to 10 carbon atoms, and a fluorine atom, n represents an integer of 0 to 12, and R¹⁰ represents an arylene group having 6 to 50 carbon atoms], wherein
a content of a repeating unit (II-A) having a three-dimensional structure represented by the following general formula (8): 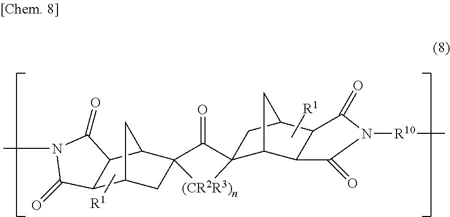
[R¹, R², R³, R¹⁰, and n in the formula (8) have the same definitions as those of R¹, R², R³, R¹⁰, and n in the general formula (7), respectively] is 40% by mol to 98% by mol relative to a total amount of the repeating unit (II),
a content of a repeating unit (II-B) having a three-dimensional structure represented by the following general formula (9): 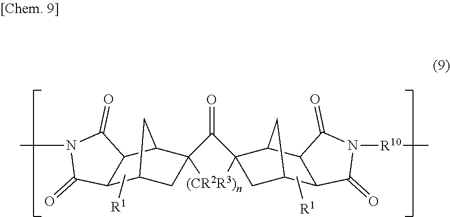
[R¹, R², R³, R¹⁰, and n in the formula (9) have the same definitions as those of R¹, R², R³, R¹⁰, and n in the general formula (7), respectively] is 2% by mol to 60% by mol relative to the total amount of the repeating unit (II), and
a summed amount of the repeating units (II-A) and (II-B) is 42% by mol or more relative to the total amount of the repeating unit (II).

4. A polyimide precursor resin solution comprising the polyimide precursor resin according to claim 2 and an organic solvent.

5. A polyimide solution comprising the polyimide according to claim 3 and an organic solvent.

6. A polyimide film that is a cured product of the polyimide precursor resin solution according to claim 4.

7. A polyimide film that is a cured product of the polyimide solution according to claim 5.
